(19) United States
(12) Patent Application Publication
(10) Pub. No.: US 2025/0012809 A1
(43) Pub. Date: Jan. 9, 2025

(54) Tissue-Specific Extracellular Vesicle Isolation and Validation

(71) Applicant: Mursla Limited, Cambridge (GB)

(72) Inventors: Tomás Miguel De Freitas Dias, Cambridge (GB); Susana Cristina Vagueiro Santos, Cambridge (GB); Ricardo Maia Pita Figueiras, Cambridge (GB)

(21) Appl. No.: 18/666,349

(22) Filed: May 16, 2024

(30) Foreign Application Priority Data
May 16, 2023 (GB) 2307304.2
May 16, 2023 (LU) LU103121

(51) Int. Cl. G01N 33/68; G01N 33/543

(52) U.S. Cl. CPC G01N 33/6872; G01N 33/543; G01N 2333/705

(57) ABSTRACT

A method of identifying a biomarker present at elevated levels in tissue of a defined category in an organism when compared to the level in at least one different category of tissue wherein the biomarker is detectable on extracellular vesicles secreted from cells of tissue of the defined category. The method comprises the steps of: (i) identifying a candidate biomarker; (ii) providing a binding agent which is capable of binding specifically to the candidate biomarker identified in step i.; (iii) detecting the presence of the candidate biomarker on an extracellular vesicle obtained from a sample of tissue of the defined category by binding the binding agent to the candidate biomarker in the sample wherein extracellular vesicles from tissue of the defined category preferentially bind the binding agent compared with extracellular vesicles from at least one different category of tissue from the organism; and (iv) selecting the candidate biomarker as a biomarker present at elevated levels in tissue of the defined category. Also provided are methods of enriching a biological sample comprising a mixture of extracellular vesicles to generate an enriched fraction of extracellular vesicles from tissue of the defined category. Also provided are methods of analysing the contents of an extracellular vesicle in an enriched fraction of extracellular vesicles from tissue of the defined category.

### RELATED APPLICATIONS

This application claims priority to United Kingdom Patent Application No. 2307304.2, filed on May 16, 2023, and claims priority to Grand Duchy of Luxembourg Application No. LU103121, filed on May 16, 2023, both of which are incorporated herein by reference in their entirety.

### FIELD OF INVENTION

The present invention relates to a method of identifying a biomarker present at elevated levels in tissue of a defined category when compared to the level in at least one different category of tissue in an organism wherein the biomarker is detectable on or in extracellular vesicles secreted from cells of tissue of the defined category. The present invention also relates to methods of isolating or enriching a biological sample comprised of extracellular vesicles from cells of tissue of a defined category, to methods of analysing a biological sample enriched in the extracellular vesicles of tissue of a defined category, and to a method of detecting one or more biomarkers on or in extracellular vesicles secreted from a tissue of a defined category. In addition, the present invention relates to a use of one or more biomarkers to generate an enriched fraction of extracellular vesicles, to a use of one or more binding agents to generate an enriched fraction of extracellular vesicles, to a kit for enriching a biological sample comprising a mixture of extracellular vesicles to generate an enriched fraction of extracellular vesicles, and method of manufacturing the kit thereof, and to an enriched fraction of extracellular vesicles.

### BACKGROUND OF INVENTION

Extracellular vesicles (EVs) are heterogeneous lipid-bilayer-encapsulated particles, such as exosomes, microvesicles, ectosomes, oncosomes, and apoptotic bodies, that are naturally secreted by cells in tissue and circulate in extracellular spaces in biofluids such as blood. EVs play an important role in regulating both cellular homeostasis and intercellular communication. In particular, they play a role in (1) removing constituents from cells that may be erroneously produced or are in excess (for example due to abnormal functions linked to an existing disease), and (2) carrying bioactive molecules to local or distant recipient cells (for example to prevent or spread a disease).

EVs are therefore enriched in biomarkers (such as proteins, sugars, nucleic acid molecules, lipids and metabolites) which reflect the molecular composition of their cellular sources. As such, EVs originating from a specific tissue act as dynamic circulating snapshots of tissue activity that can be accessed through a liquid biopsy.

It is estimated that 99.8% of total EVs in blood are originated from cells naturally residing in the blood tissue, including but not limited to platelets, erythrocytes and lymphocytes, 0.16% from adipose tissue and only 0.03% are originated from vital tissues, including but not limited to muscle, brain, skin, colon, lung, liver, heart or pancreas (Li et al., 2020). As a matter of example, it is estimated that only 0.0362% of EVs in blood are from a liver origin.

The isolation of an enriched sample of EVs which are derived from a given tissue in a biofluid is not trivial but of interest in many clinical applications, from diagnostics to therapeutics.

Conventional methods of EV isolation include ultracentrifugation, filtration, precipitation, and size-based microfluidic enrichment, and involve the bulk isolation of EVs based on their physical properties (i.e., size and/or density). However, these methods cannot distinguish between the cellular source of the EVs in the sample.

More recent methods have described the isolation of a target population of EVs using immunoaffinity-based capture techniques.

A study by Tauro et al. (2012) performed a comparative assessment of different capture methods for isolating exosomes from the culture medium of LIM1863 colorectal carcinoma cells, including ultracentrifugation, density gradient separation, and immunoaffinity capture using anti-EpCAM (a universal surface marker on epithelial cells) coated magnetic beads. Immunoaffinity capture was found to be the most effective method of isolation compared to differential centrifugation and density gradient methods. Dias et al. (2024) has reported an electro-optical platform termed Nano-Extracellular Omics Sensing for the ultrasensitive detection of EV sub-types and their target proteins. The platform merges electrical (E-NEXOS) and optical detection (O-NEXOS) for detecting and characterizing EVs.

The platform was validated in vitro following the isolation of EVs secreted from breast cancer cell lines MCF-7 and BT-474 using magnetic beads coated with anti-CD9 or anti-CD81 antibodies. O-NEXOS and E-NEXOS showed higher sensitivity than currently available technologies and that it is compatible with EVs derived from biofluids. The E-NEXOS and O-NEXOS detection methods are also disclosed in WO2019/211622, WO2021/084116 and WO2022/122768, which are incorporated herein by reference. Karimi et al. (2022) previously reported the use of O-NEXOS as a sensitive tool for the versatile detection of EVs isolated from serum and plasma samples using magnetic beads coated with antibodies against the tetraspanins CD63, CD81 or CD9 for the detection of CD41+EV sub-types.WO2021/260231 is incorporated herein by reference and discloses a two-step method of isolating target EVs.

Sun et al. (2020) reported a method for isolating hepatocellular carcinoma (HCC) EVs. The method comprised a click chemistry-mediated capture of EVs, followed by a disulfide cleavage-driven release of the EVs (termed “EV Click Chips”), in conjunction with a cocktail of antibodies targeting three HCC-associated surface markers, EpCAM, ASGPR1, and CD147. Evaluation of the method was performed in artificial plasma samples spiked with EVs derived from the HCC cell line HepG2.

In vitro artificial plasma samples and cell culture systems are simplified systems which are used to model the in vivo environment. However, such in vitro models do not accurately mimic the in vivo environment and compromise the accuracy of the biological system being investigated. In vitro models therefore do not provide an accurate model for validating that a biomarker is present at elevated levels in tissue of a defined category (e.g. liver tissue, lung tissue etc.).

In a large study, Hoshino et al. (2020) analysed 426 human plasma samples by mass-spectrometry and identified that the typical exosome markers in EVs derived from cell culture systems do not represent the typical markers found in EVs originated in vivo.

The sensitivity of the method required for the detecting a biomarker for EVs from a more complex in vivo system, such as from tissue in a biofluid, is higher than that required for the detection of a biomarker for EVs from in vitro models. As such, the prior art techniques do not make certain that the biomarkers identified are present at elevated levels in EVs from particular tissues in biofluids.

Sun et al. (2023) further reported a method of isolating HCC-derived EVs from plasma obtained from HCC patients. The method comprised incubating the sample with (1) trans-cyclooctene (TCO)-conjugated HCC-associated antibodies against HCC-associated surface markers (EpCAM, CD147, GPC3, and ASGPR1), (2) DNA conjugated anti-CD63 and anti-CD9 antibodies for EV detection, and (3)methyltetrazine [mTz]-modified microbeads to covalently link the antibodies to microbeads by click chemistry. It is described that the method is intended to be for the early detection of HCC. The technique does not involve the isolation of EVs from whole tissue or EVs originated from liver tissue in blood or blood-derivates and as such there is no confirmation of the tissue-origin of the EVs that are detected and some of the used markers are not specific or enriched in the liver.

WO2023/074541 reports a method of selecting tissue-specific EV markers, and a purification method using the selected marker for purifying EVs according to tissues from which they originate. The tissue-specific EV markers were identified using RNA sequencing data from the Human protein atlas (HPA) as a reference, whereby the condition for a molecule that is expressed in a tissue-specific manner was that it was found to be at least 4 times more expressed in the tissue of interest than in other tissues.

The problem with WO2023/074541 is that it operates on the assumption that the biomarkers that are identified using the RNA sequencing data, not only reflect the protein concentration in the different tissues but also that those proteins are internalized on EVs during their biogenesis and are located in or across the membrane of secreted EVs for their specific capture from the tissue of interest by comparison to other tissues. WO2023/074541 does not confirm that EVs of the tissue of interest are preferentially isolated or detected.

There continues to be a need for accurate methods of identifying a biomarker present at elevated levels in tissue of a defined category in an organism and isolating the EVs of a tissue of a defined category from complex samples for downstream applications. Such downstream applications include, but are not limited to collecting EVs of the tissue of the defined category from liquid biopsies which are minimally or non-invasive in contrast to traditional tissue biopsies.

Traditional tissue biopsies are not available to all patients and liquid biopsies offer a promising alternative if one is able to access information from a tissue of the defined category via blood or other biofluids.

### SUMMARY OF INVENTION

The present invention arises from the recognition that, using a detection method of sufficient sensitivity, it is possible to detect and validate a biomarker in EVs obtained from a sample of tissue of the defined category. Since there is no better representation of EVs derived from a tissue in its in vivo environment than the tissue itself, (a sample of tissue is a more accurate reflection of the in vivo environment) the present invention enables biomarkers to be identified which can be fully validated as being present at elevated levels in EVs from particular categories of tissue by comparison to at least one other tissue, ex vivo. This method enables selecting biomarkers that are truly tissue-specific or enriched in EVs derived from a targeted tissue, overcoming the existing lack of evidence when translating proteomics data to EVs, from information deposited in bioinformatics databases. As of yet, there is no bioinformatics database with tissue-EV specific biomarkers. With the present invention, such biomarkers can thereby be identified and used to characterise EVs as being from a particular category of tissue. Additionally, these biomarkers can be used to isolate EVs from a particular category of tissue in complex biofluids containing EV mixtures originated from several or all tissues in an organism.

According to a first aspect of the present invention, there is provided a method of identifying a biomarker present at elevated levels in tissue of a defined category in an organism in comparison to the level in at least one different category of tissue, wherein the biomarker is detectable on or in extracellular vesicles secreted from cells of tissue of the defined category, the method comprising the steps of:
i. identifying a candidate biomarker;
ii. providing a binding agent which is capable of binding specifically to the candidate biomarker identified in step i.;
iii. detecting the presence of the candidate biomarker on an extracellular vesicle obtained from a sample of tissue of the defined category by binding the binding agent to the candidate biomarker in the sample wherein extracellular vesicles from tissue of the defined category preferentially bind the binding agent compared with extracellular vesicles from at least one different category of tissue from the organism;
iv. selecting the candidate biomarker as a biomarker present at elevated levels in tissue of the defined category.

Preferably, step iii. comprises detecting the presence of the candidate biomarker using a technique capable of detecting the biomarker at a concentration of at least 1×10⁷ biomarkers/ml, preferably at least 1×10⁶, 1×10⁵, 1×10⁴, 1×10³, 1×10², 10 or 1 biomarkers/ml.

Alternatively, step iii. comprises detecting the presence of the candidate biomarker using a technique capable of detecting, by using the candidate biomarker, extracellular vesicles at a concentration of at least 1×10⁷ extracellular vesicles/ml, preferably at least 1×10⁶, 1×10⁵, 1×10⁴, 1×10³, 1×10², 10 or 1 extracellular vesicles/ml.

Conveniently, step iii. further comprises determining the concentration of the biomarker in the sample.

### Advantageously,

step i. comprises identifying a plurality of candidate biomarkers;
step ii. comprises providing a plurality of binding agents, each binding agent being capable of binding specifically to a respective candidate biomarker identified in step i;
step iii. comprises detecting the presence of each of the candidate biomarkers on an extracellular vesicle obtained from a sample of tissue of the defined category by binding the binding agents to the respective candidate biomarkers in the sample wherein extracellular vesicles from tissue of the defined category preferentially bind a combination of the plurality of binding agents compared with extracellular vesicles from the at least one different category of tissue from the organism; and
step iv. comprises selecting the plurality of candidate biomarkers as a combination of biomarkers present at elevated levels in tissue of the defined category.

Advantageously, step iii. further comprises contacting extracellular vesicles from the at least one different category of tissue with the or each binding agent and detecting binding of the extracellular vesicles from the at least one different category of tissue to the or each binding agent.

Preferably, extracellular vesicles from tissue of the defined category preferentially bind the or each binding agent compared with extracellular vesicles from at least 2 different categories of tissue from the organism.

Conveniently, extracellular vesicles from tissue of the defined category preferentially bind the or each binding agent compared with extracellular vesicles from all other categories of tissue from the organism.

Advantageously, step iii. further comprises the step of detecting the presence of the or each candidate biomarker on an extracellular vesicle obtained from a cultured cell line which is representative of tissue of the defined category.

Preferably, the extracellular vesicles obtained from a cultured cell line which is representative of tissue of the defined category preferentially bind the binding agent compared with extracellular vesicles obtained from a cultured cell line which is representative of at least one different category of tissue from the organism.

Conveniently, the extracellular vesicles obtained from the cultured cell line is in plasma which has been spiked with the extracellular vesicles.

Advantageously, the extracellular vesicle obtained from a sample of tissue is in plasma which has been spiked with the extracellular vesicles after the extracellular vesicles has been obtained from the sample of tissue.

Preferably, step i. comprises the steps of:
1) identifying potential candidate biomarkers expressed in the defined category of tissue;
2) identifying potential candidate biomarkers which are present in the defined category of tissue at a higher level than in at least one different category of tissue from the organism;
3) identifying potential candidate biomarkers which are transmembrane or surface proteins;
4) identifying potential candidate biomarkers which are present in extracellular vesicles; or
5) a combination of steps 1) to 4) performed in any order, and thereby identifying a candidate biomarker.

Conveniently, step i. comprises the steps of:
1) identifying potential candidate biomarkers expressed in the defined category of tissue;
2) identifying potential candidate biomarkers from within the potential candidate biomarkers identified in step 1) which are present in the defined category of tissue at a higher level than in at least one different category of tissue from the organism;
3) identifying potential candidate biomarkers from within the potential candidate biomarkers identified in step 2) which are transmembrane or surface proteins.
4) validating a candidate biomarker from within the potential candidate biomarkers identified in step 3) which is present in extracellular vesicles or validating candidate biomarkers from within the potential candidate biomarkers identified in step 3) which are present in extracellular vesicles.

Advantageously, step iii. further comprises the step of fractionating a precursor sample of tissue of the defined category enriched in extracellular vesicles in order to provide the sample of tissue of the defined category.

Preferably, the candidate biomarker is a transmembrane or surface protein.

Alternatively, the candidate biomarker is a cytosolic protein.

Conveniently, the candidate biomarker is a protein having a post-translational modification.

Alternatively, the candidate biomarker is a DNA molecule, an RNA molecule, a lipid or a post-translation modification.

Advantageously, the or each binding agent comprises an antibody, an aptamer, a lectin, a lipid-binding protein or domain, or a DNA or RNA primer or probe

Preferably, in step iii. the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene

Conveniently, the defined category of tissue is liver tissue, preferably wherein the cells from which the extracellular vesicles are secreted are hepatocytes, Kupffer cells, stellate cells or liver resident dendritic cells.

Advantageously, the or each biomarker present at elevated levels in tissue of the defined category is selected from the group consisting of ASGR1, ASGR2, TFR2, SLCO1B1, SLC38A3, TMEM56, UNC93A, SLC22A9, SLC2A2, and FXYD1, or a combination thereof, preferably wherein the or each biomarker present at elevated levels in tissue of the defined category is selected from the group consisting of ASGR1, ASGR2, TFR2, and SLCO1B1, or a combination thereof.

Preferably, the or each biomarker present at elevated levels in tissue of the defined category consists of ASGR1, ASGR2, TFR2, and SLCO1B1.

Conveniently, the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

Advantageously, each binding agent is provided in a proportion of 10-50% of the total, wherein the total mixture of binding agents is equal to 100% or wherein each binding agent is provided in equal proportions.

Preferably, the binding agent comprises a plurality of binding agents consisting of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof, and wherein the anti-ASGR1 antibody or antigen binding fragment thereof is provided in a proportion of 30-50% of the total, and each of the other antibodies or antigen binding fragments thereof is provided in a proportion of 10-30% of the total, wherein the total mixture of the binding agents is equal to 100%.

Alternatively, the defined category of tissue is blood tissue.

Advantageously, the biomarker is specific for tissue of the defined category or wherein the combination of biomarkers is specific for tissue of the defined category.

Preferably, step iii. further comprises the step of detecting the binding of the or each binding agent to the respective candidate biomarker on or in an extracellular vesicle.

Conveniently, the sample of tissue is healthy tissue or diseased tissue.

According to a second aspect of the present invention, there is provided a method of enriching a biological sample comprising a mixture of extracellular vesicles comprising the steps of:
a) identifying one or more biomarkers present at elevated levels in a tissue by performing the method according to the first aspect of the present invention;
b) capturing extracellular vesicles in the biological sample, wherein the extracellular vesicles display the or each biomarker identified in step a), to generate an enriched fraction of extracellular vesicles from tissue of the defined category. Advantageously, the mixture of extracellular vesicles comprises extracellular vesicles from tissue of the defined category and extracellular vesicles from at least one different category of tissue from the organism.

Conveniently, the extracellular vesicles are captured in step b) using one or more binding agents which are capable of binding specifically to the or each biomarker.

Preferably, wherein step b) further comprises isolating the extracellular vesicles displaying the or each biomarker identified in step a) which are captured using the or each biomarker.

Conveniently, the isolated extracellular vesicles are intact extracellular vesicles.

Advantageously, the method further comprises the step of releasing the contents of the captured or isolated extracellular vesicles.

According to a third aspect of the present invention, there is provided a method of analysing a biological sample comprising a mixture of extracellular vesicles comprising the steps of:
a) identifying a biomarker present at elevated levels in a tissue by performing the method according to the first aspect of the present invention;
b) capturing extracellular vesicles in the biological sample, wherein the extracellular vesicles display the or each biomarker identified in step a), to generate an enriched fraction of extracellular vesicles; and
c) analysing the contents of an extracellular vesicle in the enriched fraction obtained in step b).

Conveniently, the mixture of extracellular vesicles comprises extracellular vesicles from tissue of the defined category and extracellular vesicles from at least one different category of tissue from the organism.

Preferably, the extracellular vesicles are captured in step b) using one or more binding agents which are capable of binding specifically to the or each biomarker.

Advantageously, step b) further comprises isolating the extracellular vesicles displaying the or each biomarker identified in step a) which are captured using the or each biomarker.

Preferably, the isolated extracellular vesicles are intact extracellular vesicles.

Conveniently, the method further comprises the step of releasing the contents of the captured or isolated extracellular vesicles.

Advantageously, the biological sample is a biofluid, preferably blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

Preferably, step b) comprises capturing extracellular vesicles in the biological sample using the or each binding agent.

According to a fourth aspect of the present invention, there is provided a method of enriching a biological sample comprising a mixture of extracellular vesicles comprising the step of:
capturing extracellular vesicles in the biological sample, wherein the extracellular vesicles display one or more biomarkers, to generate an enriched fraction of extracellular vesicles,
wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

According to a fifth aspect of the present invention, there is provided a method of analysing a biological sample comprising a mixture of extracellular vesicles comprising the steps of:
a) capturing extracellular vesicles in the biological sample, wherein the extracellular vesicles display one or more biomarkers, to generate an enriched fraction of extracellular vesicles; and
b) analysing the contents of an extracellular vesicle in the enriched fraction obtained in step a),
wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

Conveniently, the step of capturing extracellular vesicles comprises capturing extracellular vesicles based on the extracellular vesicles displaying one or more biomarkers and wherein the one or biomarkers consist of ASGR1, ASGR2, TFR2, and SLCO1B1.

Advantageously, the extracellular vesicles are captured using one or more binding agents which are capable of binding specifically to the or each biomarker. Preferably, the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

Conveniently, each binding agent is provided in a proportion of 10-50% of the total, wherein the total mixture of binding agents is equal to 100% or wherein each binding agent is provided in equal proportions.

Advantageously, the binding agent comprises a plurality of binding agents consisting of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof, and wherein the anti-ASGR1 antibody or antigen binding fragment thereof is provided in a proportion of 30-50% of the total, and each of the other antibodies or antigen binding fragments thereof is provided in a proportion of 10-30% of the total, wherein the total mixture of the binding agents is equal to 100%.

Preferably, the mixture of extracellular vesicles comprises extracellular vesicles from tissue of a defined category and extracellular vesicles from at least one different category of tissue from the organism, and wherein the or each biomarker is present at elevated levels in the tissue of the defined category.

Conveniently, the method further comprises isolating the extracellular vesicles displaying the or each biomarker which are captured using the or each biomarker.

Advantageously, the isolated extracellular vesicles are intact extracellular vesicles.

Preferably, the method further comprises the step of releasing the contents of the captured or isolated extracellular vesicles.

Conveniently, the biological sample is a biofluid, preferably blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

Advantageously, the step of capturing extracellular vesicles in the biological sample comprises capturing extracellular vesicles in the biological sample using the or each binding agent.

According to a sixth aspect of the present invention, there is provided a use of one or more biomarkers displayed on extracellular vesicles to generate an enriched fraction of extracellular vesicles, wherein the enriched fraction of extracellular vesicles is secreted from tissue of a defined category in an organism, wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

Preferably, the biomarkers consist of ASGR1, ASGR2, TFR2, and SLCO1B1.

Conveniently, the enriched fraction of extracellular vesicles is enriched from a biological sample comprising a mixture of extracellular vesicles.

Advantageously, the biological sample is a biofluid, preferably blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

According to a seventh aspect of the present invention, there is provided a use of one or more binding agents to generate an enriched fraction of extracellular vesicles, wherein the enriched fraction of extracellular vesicles is secreted from tissue of a defined category in an organism, wherein the or each binding agent is capable of binding specifically to a biomarker displayed on the extracellular vesicles, wherein the or each binding agent is selected from the group consisting of an anti-ASGR1 antibody or antigen binding fragment thereof, an anti-ASGR2 antibody or antigen binding fragment thereof, an anti-TFR2 antibody or antigen binding fragment thereof, an anti-SLCO1B1 antibody or antigen binding fragment thereof, an anti-SLC38A3 antibody or antigen binding fragment thereof, an anti-TMEM56 antibody or antigen binding fragment thereof, an anti-UNC93A antibody or antigen binding fragment thereof, an anti-SLC22A9 antibody or antigen binding fragment thereof, an anti-SLC2A2 antibody or antigen binding fragment thereof, and an anti-FXYD1 antibody or antigen binding fragment thereof, or a combination thereof, preferably an anti-ASGR1 antibody or antigen binding fragment thereof, an anti-ASGR2 antibody or antigen binding fragment thereof, an anti-TFR2 antibody or antigen binding fragment thereof, or an anti-SLCO1B1 antibody or antigen binding fragment thereof, or a combination thereof.

Preferably, the enriched fraction of extracellular vesicles is enriched from a biological sample comprising a mixture of extracellular vesicles.

According to an eighth aspect of the present invention, there is provided a method of detecting one or more biomarkers on or in extracellular vesicles secreted from a tissue of a defined category, wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

Conveniently, the method comprises a plurality of biomarkers comprising ASGR1, ASGR2, TFR2, and SLCO1B1.

Advantageously, the or each biomarker is detected by the binding of one or more binding agents, wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is

TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

Preferably, the or each biomarker is detected on or in extracellular vesicles secreted from a tissue of a defined category in a biological sample comprising a mixture of extracellular vesicles.

According to an ninth aspect of the present invention, there is provided a kit for enriching a biological sample comprising a mixture of extracellular vesicles to generate an enriched fraction of extracellular vesicles, wherein the enriched fraction of extracellular vesicles is secreted from tissue of a defined category in an organism, wherein the kit comprises one or more binding agents which are capable of binding specifically to a biomarker present at elevated levels in tissue of the defined category, and wherein the or each binding agent is selected from the group consisting of an anti-ASGR1 antibody or antigen binding fragment thereof, an anti-ASGR2 antibody or antigen binding fragment thereof, an anti-TFR2 antibody or antigen binding fragment thereof, an anti-SLCO1B1 antibody or antigen binding fragment thereof, an anti-SLC38A3 antibody or antigen binding fragment thereof, an anti-TMEM56 antibody or antigen binding fragment thereof, an anti-UNC93A antibody or antigen binding fragment thereof, an anti-SLC22A9 antibody or antigen binding fragment thereof, an anti-SLC2A2 antibody or antigen binding fragment thereof, and an anti-FXYD1 antibody or antigen binding fragment thereof, or a combination thereof, preferably an anti-ASGR1 antibody or antigen binding fragment thereof, an anti-ASGR2 antibody or antigen binding fragment thereof, an anti-TFR2 antibody or antigen binding fragment thereof, or an anti-SLCO1B1 antibody or antigen binding fragment thereof, or a combination thereof.

Conveniently, the mixture of extracellular vesicles comprises extracellular vesicles from tissue of the defined category and extracellular vesicles from at least one different category of tissue from the organism.

Advantageously, the biological sample is a biofluid, preferably blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

Preferably, the or each binding agent consists of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof.

Conveniently, the method, use or kit comprises a plurality of binding agents consisting of the anti-ASGR1 antibody, the anti-ASGR2 antibody, the anti-SLCO1B1 antibody and the anti-TFR2 antibody.

Advantageously, the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene.

Preferably, each binding agent is provided in a proportion of 10-50% of the total, wherein the total mixture of binding agents is equal to 100% or wherein each binding agent is provided in equal proportions.

Conveniently, the use, method or kit comprises a plurality of binding agents consisting of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof, and wherein the anti-ASGR1 antibody or antigen binding fragment thereof is provided in a proportion of 30-50% of the total, and each of the other antibodies or antigen binding fragments thereof is provided in a proportion of 10-30% of the total, wherein the total mixture of binding agents is equal to 100%.

According to a tenth aspect of the present invention, there is provided an enriched fraction of extracellular vesicles, the fraction being enriched with extracellular vesicles secreted from tissue of a defined category in an organism, which extracellular vesicles display one or more biomarkers selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

Advantageously, the extracellular vesicles secreted from tissue of a defined category in an organism display a plurality of biomarkers comprising ASGR1, ASGR2, TFR2, and SLCO1B1.

Preferably, the defined category of tissue is liver tissue.

According to an eleventh aspect of the present invention, there is provided a method of manufacturing the kit according to the ninth aspect of the present invention, wherein the method comprises colocating the or each binding agent.

### Definitions

The terms “biomarker” or “biological marker” are used interchangeably herein to refer to a naturally occurring molecule, such as a protein, a post-translational modification on a protein, a gene, a nucleic acid molecule (e.g. DNA or RNA), an epigenetic modification on a nucleic acid molecule, a lipid or a metabolite, or a part thereof, which is a measurable indicator of a particular state. The term “candidate biomarker” is here interchangeably referred to as a “candidate tissue-specific biomarker or “tissue-enriched biomarker” that are selected for evaluation as a biomarker which is present at elevated levels in tissue of the defined category and can be used for the capture of extracellular vesicles from the tissue of the defined category.

The term “present at elevated levels” as used herein refers to the presence of a biomarker being elevated in the tissue of a defined category with respect to the presence of the biomarker in at least one different category of tissue.

The terms “polypeptide”, “peptide” and “protein” are used interchangeably herein to refer to a polymer of amino acid residues. The terms apply to amino acid polymers in which one or more amino acid residues is a modified residue, or a non-naturally occurring residue, such as an artificial chemical mimetic of a corresponding naturally occurring amino acid, as well as to naturally occurring amino acid polymers. In some embodiments, the phrase “transmembrane protein” as used herein refers to a type of integral membrane protein that spans the entirety of the plasma membrane. In some embodiments, the phrase “surface protein” as used herein refers to refers to a type of integral membrane protein that is embedded in the plasma membrane and is exposed to the external side of the plasma membrane.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code, as well as those modified after translation in cells (e.g. hydroxyproline, gamma-carboxyglutamate, and O-phosphoserine).

The term “post-translational modification” as used herein refers to a biochemical modification of an amino acid in a protein following protein biosynthesis. These post-translational modifications include but are not limited to acetylation, glycosylation, hydroxylation, lipidation, methylation, nitrosylation, phosphorylation, proteolysis, and ubiquitination.

The terms “gene”, “polynucleotides”, and “nucleic acid molecules” are used interchangeably herein to refer to a polymer of multiple nucleotides. The nucleic acid molecules may comprise naturally occurring nucleic acids (i.e. DNA or RNA) or may comprise artificial nucleic acids such as peptide nucleic acids, morpholin and locked nucleic acid as well as glycol nucleic acid and threose nucleic acid.

The term “epigenetic modification” as used herein refers to a biochemical modification of a nucleic acid molecule. These epigenetic modifications include but are not limited to DNA methylation.

The term “oligonucleotide” as used herein refers to a single-stranded polymer of nucleotides.

The term “nucleotide” as used herein refers to naturally occurring nucleotides and synthetic nucleotide analogues that are recognised by cellular enzymes. A nucleotide is the basic building block of nucleic acids (i.e. DNA or RNA) consists of a sugar molecule (either deoxyribose in DNA or ribose in RNA) attached to a phosphate group and a nitrogen-containing base. The bases used in DNA are adenine (A), cytosine (C), guanine (G), and thymine (T) in DNA or uracil (U) in RNA. Double-stranded DNA and RNA is formed from two complementary strands of nucleotides which are held together by hydrogen bonds between pairs of nucleotides (G with C, and A with T or U). In this specification, the percentage of a sequence which is “complementary” to another sequence (i.e. a target sequence) is determined using sequencing methods such as

EMBOSS Needle Pairwise Sequence Alignment (Rice et al., Trends Genet. 2000 June; 16 (6): 276-7; Nucleic Acids Res. 2019 Jul. 2;47 (W1): W636-W641) using default parameters. In particular, EMBOSS Needle can be accessed on the internet using the URL: https://www.ebi.ac.uk/Tools/psa/emboss_needle/. In particular, it is known in the literature as to how to ascertain an oligonucleotide sequence that is 100% complementary to an oligonucleotide sequence, for example using tools such as EMBOSS revseq which be the can accessed online using URL: https://www.bioinformatics.nl/cgi-bin/emboss/revseq. Sequencing methods such as EMBOSS Needle Pairwise Sequence Alignment can then be used to ascertain a particular level of percentage sequence identity between the complementary oligonucleotide sequence and the target oligonucleotide sequence.

The term “organism” as used herein refers to an individual animal, plant, or single-celled life form.

The term “tissue” as used herein refers to a group or layer of cells that possess a similar structure and perform a specific function. A tissue may also contain groups or layers of different cells that work together to perform a specific function. The term “tissue” includes but is not limited to connective tissue (including but not limited to loose connective tissue, adipose tissue, dense fibrous connective tissue, elastic connective tissue, cartilage, osseous tissue, and blood), epithelial tissue (including but not limited to simple squamous, stratified squamous, simple cuboidal, stratified cuboidal, simple columnar, stratified columnar, pseudostratified columnar and transitional epithelia, and urothelium), muscle tissue (including but not limited to cardiac tissue, smooth tissue, and skeletal tissue), nervous tissue (including but not limited to neurons and neuroglia) and liver tissue (including but not limited to hepatocytes, Kupffer cells, endothelial cells and hepatic stellate cells). The phrase “tissue of the defined category” refers to a tissue found in or obtained from a particular organ or organ system. In one embodiment the tissue of the defined category is liver tissue or blood tissue.

The term “extracellular vesicle” as used herein refers to heterogeneous lipid-bilayer-encapsulated particles that are naturally secreted by cells. EVs are non-self-replicating and circulate in extracellular spaces in biofluids. The term “extracellular vesicle” consists of variety of subtypes, including but not limited to exosomes, microvesicles, ectosomes, oncosomes, and apoptotic bodies. The term “extracellular vesicle” is also used interchangeably with the term “extracellular nanoparticles” and “non-vesicular extracellular nanoparticles” which include but are not limited to supermeres and exomers.

The term “binding agent” as used herein refers to a naturally occurring and synthetic molecule which is capable of binding specifically to a target molecule on a target entity. In some embodiments, the term “binding agent” refers to a molecule which is capable of binding specifically to the candidate biomarker on or in EVs. The phrase “binding specifically” or “binds specifically” refers to the preferential binding of the binding agent to the target molecule in comparison to non-target molecules. The term “binds specifically” means that the antibodies have substantially greater affinity for their target polypeptide than their affinity for other related polypeptides. By “substantially greater affinity” is meant that there is a measurable increase in the affinity for the target polypeptide of the invention as compared with the affinity for other related polypeptide. In some embodiments, the affinity is at least 1.5-fold, 2-fold, 5-fold, 10-fold, 100-fold, 10³-fold, 10⁴-fold, 10⁵-fold, 10⁶-fold or greater for the target polypeptide. In some embodiments, the antibodies bind with high affinity, with a dissociation constant of 10-4M or less, 10-7M or less, 10-9M or less; or subnanomolar affinity (0.9, 0.8, 0.7, 0.6, 0.5, 0.4, 0.3, 0.2, 0.1 nM or even less). For example, the binding specificity of a binding agent can be tested using an in vitro binding assay using purified proteins.

The term “antigen binding fragment” of an antibody means a partial fragment of an antibody having an antigen-binding activity and includes Fab, F (ab′) 2, scFv and the like. The term also encompasses Fab′ which is a monovalent fragment in a variable region of an antibody obtained by treating F (ab′) 2 under reducing conditions. However, the term is not limited to these molecules as long as the fragment has a binding affinity for an antigen. Further, these antigen-binding fragments include not only a fragment obtained by treating a full-length molecule of an antibody protein with an appropriate enzyme, but also a protein produced in an appropriate host cell using a genetically modified antibody gene.

The term “capture” as used herein refers to the immobilisation of a target entity relative to a substrate. In some embodiments, the term “capture” refers to the immobilisation of EVs displaying the candidate biomarker in a sample, preferably a biological sample, by the binding agent.

The term “biological sample” as used herein refers to a sample obtained from an organism. In one embodiment, the biological sample is a sample of tissue obtained by biopsy (e.g. a tissue biopsy). In one embodiment, the biological sample (e.g. tissue biopsy) is snap frozen. In an alternative embodiment, the biological sample is a biofluid. In one embodiment, the biological sample is a sample of whole blood, plasma or serum. In some embodiments, the biological sample comprises EVs. In embodiments where the biological sample is a biofluid, the biofluid comprises circulating EVs.

The terms “biofluid” or “biological fluid” are used interchangeably herein to refer to a liquid obtained from an organism, whereby the liquid helps transport cells, particles, vesicles and nutrients and expels waste from cells.

The term “plasma” as used herein refers to the liquid component of blood excluding the red blood cells, white blood cells and platelets, in the presence of an anticoagulant.

The term “serum” as used herein refers to the liquid component of blood excluding the red blood cells, white blood cells and platelets, after the blood has clotted.

The terms plasma and serum may be naturally occurring or artificial.

The term “artificial plasma” as used herein refers to a liquid which is assembled to mimic the characteristics of human plasma (Khan et al., 2020).

The term “isolation” as used herein refers to the separation of a target entity from a sample containing at least one other component. In some embodiments, the term “isolation” refers to the separation of EVs displaying the candidate biomarker in a sample, preferably a biological sample, via capture of the EVs using the binding agent.

The term “enriched fraction” as used herein refers to the separated fraction of a sample following isolation which is primarily composed of the target entity. In some embodiments, the term “enriched fraction” refers to the fraction of a sample, preferably a biological sample, which, following isolation, is primarily composed of EVs.

The terms “detecting” or “detection” are used herein to refer to a measurable readout. In some embodiments, the term “detection” refers to a measurable readout of the presence of the candidate biomarker. In some embodiments, the level of detection of the presence of the candidate biomarker is quantified. In some embodiments, the presence of the candidate biomarker is detected at a desired sensitivity defined by detecting the biomarker at a concentration in units of biomarkers/ml. In some embodiments, the presence of the candidate biomarker is detected at a concentration in units of EVs/mL. In some embodiments, the presence of the candidate biomarker is inferred from the signal output obtained with the technique used for detection.

The term “control” as used herein refers to a comparison against which the method can be evaluated. In one embodiment, the control is a negative control. In an alternative embodiment, the control is a positive control.

The terms “cancer” and “tumor” as used herein refer to the presence of cells in a subject that exhibit new, abnormal and/or uncontrolled proliferation. In one embodiment, the cells have the capacity to invade adjacent tissues and/or to spread to other sites in the body (i.e. the cells are capable of metastasis). In one embodiment, the cancer cells are in the form of a tumor (i.e. an abnormal mass of tissue). The term “tumor” as used herein includes both benign and malignant neoplasms. In one embodiment, the cancer is liver cancer. In some embodiments, the liver cancer is HCC.

The term “in vivo” (Latin for “within the living”) as used herein refers to experiments performed in a whole, living organism. The term “in vitro” (Latin for “in glass”) as used herein refers to experiments performed on biological material cultured or stored in an artificial receptacle. The term “ex vivo” (Latin for “out of the living”) as used herein refers to experiments performed on biological material taken from an organism.

As used herein, each of the biomarkers ASGR1, ASGR2, TFR2, SLCO1B1, SLC38A3, TMEM56, UNC93A, SLC22A9, SLC2A2 or FXYD1 has, in preferred embodiments, the amino acid sequence corresponding to the respective Uniprot ID number shown in any one of Tables 1 to 3, the respective amino acid sequences are herein incorporated by reference.

### BRIEF DESCRIPTION OF THE FIGURES

Embodiments of the invention will now be described with reference to the following figures in which:

FIG. 1 is a heatmap of the protein expression profile of 88 candidate biomarkers for liver tissue extracted from ProteomicsDB.

FIGS. 2A, 2B and 2C are a heatmap of the protein expression profile of 88 candidate biomarkers for liver tissue extracted from Human Proteome Map.

FIG. 3 is a heatmap of the protein expression profile of 11 candidate biomarkers for liver tissue extracted from ProteomicsDB.

FIG. 4 is a heatmap of the protein expression profile of 11 candidate biomarkers for liver tissue extracted from Human Proteome Map.

FIG. 5 is a graph summarising the CD63-detection of EVs isolated from the HCC cell line HepG2, the breast cancer cell line MCF-7 (1), the breast cancer cell line BT-474 (2) or a buffer control using antibodies targeting each of the 11 biomarkers as binding agents. ASGR1 (antibody 1; ab1) and ASGR1 (antibody 2; ab2) refer to two different antibodies targeting the ASGR1 biomarker.

FIG. 6 is a graph summarising the CD63-detection of EVs isolated from healthy liver tissue, HCC tissue, kidney tissue, lung tissue or a buffer control using binding agents targeting each of the 10 biomarkers.

FIG. 7 is a graph summarising the CD63-detection of EVs isolated from human plasma samples derived from blood taken from five healthy individuals using binding agents targeting each of the biomarkers ASGR1, ASGR2, TFR2 and SLCO1B1 and an isotype control.

FIG. 8 is a graph summarising the CD63-detection of EVs isolated from human plasma from a healthy individual, HCC patient, two liver patients and buffer control using a liver cocktail combining a combination of the binding agents targeting ASGR1, ASGR2, TFR2 and SLCO1B1 at a final ratio of 4:2:2:2 and an isotype control.

FIG. 9 is a graph summarising the signal-to-noise ratio (SNR) of the CD63-detection of EVs isolated from HCC tissue, cirrhosis tissue, healthy liver tissue, lung tissue, kidney tissue using a binding agent targeting CD63.

FIG. 10 is a graph summarising the total spectrum count of 7 liver-specific proteins detected in EVs by mass spectrometry following collection of the EVs from human plasma, isolation using the cocktail of binding agents targeting ASGR1, ASGR2, TFR2 and SLCO1B1 and eluted using glycine or DNase, or lysed directly on magnetic beads with RIPA.

FIG. 11 is a graph summarising the GO cellular component analysis of the genes detected in EVs by mass spectrometry following collection of the EVs from human plasma, isolation using the cocktail of binding agents targeting ASGR1, ASGR2, TFR2 and SLCO1B1 and eluted using glycine or DNase, or lysed directly on magnetic beads with RIPA.

FIG. 12 is a graph summarising the log 2 intensity of 13 classical EV markers detected in EVs by mass spectrometry following collection of the EVs from human plasma, isolation using the cocktail of binding agents targeting ASGR1, ASGR2, TFR2 and SLCO1B1 and lysis using RIPA or SDC buffer.

FIGS. 13A and 13B are a graph summarising the log 2 intensity of 63 liver-specific markers detected in EVs by mass spectrometry following collection of the EVs from human plasma, isolation using the cocktail of binding agents targeting ASGR1, ASGR2, TFR2 and SLCO1B1 and lysis using RIPA or SDC buffer.

FIG. 14A is a graph summarising the GO cellular component analysis of the genes detected in EVs by mass spectrometry following collection of the EVs from human plasma, isolation using the cocktail of binding agents targeting ASGR1, ASGR2, TFR2 and SLCO1B1 and lysis using RIPA buffer.

FIG. 14B is a graph summarising the GO cellular component analysis of the genes detected in EVs by mass spectrometry following collection of the EVs from human plasma, isolation using the cocktail of binding agents targeting ASGR1, ASGR2, TFR2 and SLCO1B1 and lysis using SDC buffer.

FIG. 15 is a graph summarising the total number of miRNA IDs detected in EVs by RNA sequencing following collection of the EVs from human plasma, and isolation using a binding agent targeting ASGR1 only or the cocktail of binding agents targeting ASGR1, ASGR2, TFR2 and SLCO1B1 in combination (TOP4). Two methods of RNA library preparation were used in two independent runs for each: RealSeq (Sequencing Method 1) and NEXTFLEX v4 (Sequencing Method 2).

FIG. 16 shows the Biological Natural Language Processing (BioNLP) analysis of the RNA sequencing data obtained from EVs following collection of the EVs from human plasma, isolation of the EVs by two protocols. Group A was directly processed for RNA sequencing (Protocol A). Group B was isolated using the cocktail of binding agents targeting ASGR1, ASGR2, TFR2 and SLCO1B1 before being processed for RNA sequencing (Protocol B).

### DETAILED DESCRIPTION OF THE INVENTION

A method of identifying a biomarker on EVs specific to a tissue of a defined category for the isolation of the tissue-specific EVs from an organism

The present invention provides a method of identifying a biomarker present at elevated levels in tissue of a defined category in an organism in comparison to the level in at least one different category of tissue. The biomarker is detectable on or in extracellular vesicles secreted from cells of tissue of the defined category. The method comprises the steps of:
V. identifying a candidate biomarker;
vi. providing a binding agent which is capable of binding specifically to the candidate biomarker identified in step i.;
vii. detecting the presence of the candidate biomarker on an EV obtained from a sample of tissue of the defined category by binding the binding agent to the candidate biomarker in the sample wherein extracellular vesicles from tissue of the defined category preferentially bind the binding agent compared with extracellular vesicles from at least one different category of tissue from the organism; and
viii. selecting the candidate biomarker as a biomarker present at elevated levels in tissue of the defined category.

### Extracellular Vesicles

Extracellular vesicles are heterogeneous lipid-bilayer-encapsulated particles that are naturally secreted from all types of cells. Extracellular vesicles play a role in removing components from cells that may be erroneously produced or are in excess and/or transporting cellular components either locally or to distant sites via the circulation. These extracellular vesicles are thus enriched in proteins, nucleic acid molecules (e.g. DNA or RNA), lipids and metabolites which mirror the molecular composition of their parental cells and/or tissue of the defined category. As such, the proteins, nucleic acid molecules, lipids and metabolites which are detectable on or in extracellular vesicles have utility as candidate biomarkers of the cells and/or tissue of the defined category from which the EV was secreted.

In some embodiments, the EV is an exosome, a microvesicle, an ectosome, an oncosome or an apoptotic body.

### Candidate Biomarker

In some embodiments, the candidate biomarker is a protein, a DNA molecule, an RNA molecule, a lipid, a complex sugar, or part of a protein, a DNA molecule, a RNA molecule, a lipid, a post-translational modification, or a metabolite, which is detectable on or in extracellular vesicles. In some embodiments, the candidate biomarker is detectable on the surface of extracellular vesicles. In embodiments where the candidate biomarker is a protein, or part thereof, preferably, the candidate biomarker is a transmembrane protein, surface protein or a cytosolic protein, or a part thereof. Preferably, the candidate biomarker is a transmembrane or surface protein, or a part thereof. In embodiments where the candidate biomarker is part of a protein, preferably the biomarker is one epitope on the protein. In embodiments where the candidate biomarker is a protein, or a part of a protein, the protein may have a post-translational modification, such as acetylation, glycosylation, hydroxylation, lipidation, methylation, nitrosylation, phosphorylation, proteolysis, and ubiquitination. In some embodiments, the candidate biomarker is a post-translational modification on a protein. In some embodiments, the candidate biomarker is an epigenetic modification on a nucleic acid molecule, such as methylation.

In some embodiments, the candidate biomarker is a molecule present in the cytosol of extracellular vesicles. In embodiments where the candidate biomarker is a cytosolic molecule, the step of detecting the presence of the candidate biomarker comprises a step of permeabilising the membrane of extracellular vesicles. Preferably, the permeabilisation step comprises the use of detergents, such as Triton-X, NP-40, saponin and others, sonication or electroporation.

### Binding Agent

The binding agent is one member of a binding pair capable of binding specifically to the candidate biomarker, which is the other member of the binding pair, and which is detectable on or in of EVs secreted from cells of tissue of the defined category. In some embodiments, or each binding agent, comprises an antibody, an antigen-binding fragment of an antibody (e.g. a Fab fragment of a F (ab′) 2 fragment), an aptamer, a lectin, a lipid-binding protein or domain, a DNA oligonucleotide or an RNA oligonucleotide. In some embodiments, the binding agent is a DNA or RNA primer or probe. In embodiments where the binding agent is a DNA oligonucleotide or an RNA oligonucleotide, it is preferable that the candidate biomarker is also an DNA oligonucleotide or an RNA oligonucleotide and the binding agent comprises a region having a sequence complementary to the candidate biomarker. In some of these embodiments, the binding agent comprises a region having a nucleic acid sequence which is 100% complementary to the nucleic acid sequence of the candidate biomarker.

In some of these embodiments, the binding agent comprises a region having a nucleic acid sequence which is at least 50%, at least 60%, at least 70%, at least 80%, at least 90% or at least 95% complementary to the nucleic acid sequence of the candidate biomarker. Preferably, the or each binding agent comprises an antibody or an antigen-binding fragment.

An antibody, also known as immunoglobulin, consists of four polypeptides, comprising two identical light chains and two identical heavy chains, joined by noncovalent interactions and disulfide bonds to form a flexible Y-shaped structure. Each of the four chains has a variable region at its amino terminus, which contributes to the antigen-binding site, and a constant region at its carboxy terminus, which determines the isotype. Antibodies are divided into five major classes of immunoglobulin, IgM, IgG, Iga, IgD, and IgE, based on their constant region structure and immune function.

The fragment antigen binding (Fab) region is composed of one constant and one variable domain from each heavy and light chains of the antibody. The variable region is further subdivided into hypervariable and framework regions. Each of the light and heavy chains contain three hypervariable loops, also known as complementarity determining regions (CDRs) and four framework regions. The six CDRs exhibit a hypervariable amino acid composition and are involved in determining the antigen binding specificity of the antibody.

In embodiments where the binding agent comprises an antigen-binding fragment, preferably, the antigen-binding fragment is a F (ab′) 2 fragment, Fab′ fragment, Fab fragment or a variable region.

Antibodies are normally produced by B cells, which are part of the immune system, when an organism's immune system encounters a foreign molecule (typically a protein). Antibodies can be generated which are specific for a candidate biomarker by repeated immunisation of a suitable animal, such as rabbit, goat, donkey or sheep with the candidate biomarker. Antigen-binding fragments can then be generated by antibody fragmentation, such as by enzyme-mediated digestion of the antibody.

In some embodiments, the or each binding agent is provided attached to a substrate. Preferably, the substrate is a structure or a particle. In embodiments where the substrate is a structure, the structure is immobile with respect to any surrounding sample. In embodiments where the substrate is a particle, the particle is mobile within a surrounding sample. Preferably the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene. Most preferably the substrate comprises a magnetic bead. In preferred embodiments, the binding agent comprises an antibody and the substrate comprises a magnetic bead (i.e. antibody-labelled magnetic bead).

### Use of the Method

As discussed above, the present invention provides a method of identifying a biomarker on EVs specific to a tissue of a defined category for the isolation of the tissue-specific EVs from an organism, such as from the biofluid of an organism.

The presence of the candidate biomarker on an EV obtained from a sample of tissue of the defined category is detected by the binding of the binding agent to the candidate biomarker in the sample. In particular, due to the presence of the candidate biomarker, the EVs from tissue of the defined category preferentially bind the binding agent compared with EVs from at least one different category of tissue from the organism. The defined category of tissue refers to a tissue found in or obtained from a particular organ or organ system. The at least one different category of tissue from the organism refers to a tissue which is not the defined category of tissue.

In one embodiment, a biological sample, such as a liver tissue, is taken from an individual in order to identify biomarkers present at elevated levels in a tissue of a defined category in the individual. A candidate biomarker is selected by a process described in more detail below. The biological sample comprises EVs secreted from cells of tissue of the defined category. The biological sample is incubated with a binding agent (e.g. an antibody) which is capable of binding specifically to the candidate biomarker, the binding agent being attached to a substrate such as a magnetic bead.

After the period of incubation, the candidate biomarker which is present on the EVs in the biological sample binds to the binding agent, such that EVs which present the candidate biomarker on their surface become attached to the substrate via the binding agent. The EVs which present the candidate biomarker on their surface can thereby be recovered from the biological sample, such as by washing of the samples containing magnetic bead-captured EVs by the washing in a magnetic field.

The binding of the binding agent to the candidate biomarker molecules is detected by the recovery of EVs from the biological sample. The recovered EVs are detected by a detection agent which is specific for a global biomarker present at elevated levels in EVs (e.g. a biotinylated anti-CD63 antibody), such that the detection agent binds to the global biomarker on the recovered EVs. The detection agent can be detected using a reporter assay (such as a florescence assay, e.g. using horseradish peroxidase (HRP) conjugated to streptavidin). The detection of a signal by the reporter assay confirms the presence of the candidate biomarker on the surface of an EV secreted from cells of tissue of the defined category.

The method is repeated, in parallel or sequentially, using a biological sample from at least one different category of tissue from the organism, such that the biological sample comprises EVs secreted from cells of the different category of tissue. The candidate biomarker is selected as a biomarker present at elevated levels in tissue of the defined category when it is it detected that the binding agent preferentially binds EVs from tissue of the defined category compared with EVs from at least one different category of tissue.

In particular, the candidate biomarker is selected as a biomarker present at elevated levels in tissue of the defined category when the signal detected by the reporter assay is higher in the biological sample taken from the tissue of a defined category in the individual compared with the biological sample from at least one different category of tissue.

In some embodiments, the candidate biomarker is not selected as a biomarker present at elevated levels in tissue of the defined category when the signal detected by the reporter assay is the same or lower in the biological sample taken from the tissue of a defined category in the individual compared with the biological sample from at least one different category of tissue.

In embodiments where the EVs from tissue of the defined category preferentially bind the binding agent compared with EVs from at least one different category of tissue from the organism, the candidate biomarker is selected as a biomarker present at elevated levels in tissue of the defined category.

For example, in embodiments where the tissue of the defined category is liver tissue, due to the presence of a candidate biomarker on the liver-derived EVs, EVs from liver tissue preferentially bind to the binding agent compared with EVs from non-liver tissue, such as kidney tissue or breast tissue. As such, the candidate biomarker is selected as a biomarker present at elevated levels in liver tissue.

In some embodiments, the defined category of tissue is brain, breast, liver, lung, bladder, kidney, heart, gall bladder, pancreas, stomach, intestine, skin, or blood tissue. It is preferred that the defined category of tissue is liver tissue or blood tissue.

In embodiments where the defined category of tissue is liver tissue, preferably the cells from which the EVs are secreted are hepatocytes, Kupffer cells, stellate cells or liver-resident dendritic cells. Most preferably, the cells from which the EVs are secreted are hepatocytes.

In some embodiments the defined category of tissue is blood tissue.

In some embodiments, the step of detecting the presence of the candidate biomarker on an EV further comprises the step of detecting the binding of the binding agent (or the binding of each binding agent) to the respective candidate biomarker on or in an extracellular vesicle.

In some embodiments, the method comprises detecting the presence of the candidate biomarker using a technique capable of detecting the biomarker at a concentration of at least 1×10⁷ biomarkers/ml, at least 1×10⁶, at least 1×10⁵, at least 1×10⁴, at least 1×10³, at least 1×10², at least 10 or at least 1 biomarkers/ml. Preferably, at least 1×10⁶, at least 1×10⁵, at least 1×10⁴, at least 1×10³, at least 1×10², at least 10 or at least 1 biomarkers/ml. In some embodiments, the method comprises detecting the presence of the candidate biomarker using a technique capable of detecting, by using the candidate biomarker, extracellular vesicles at a concentration of at least 1×10⁷ extracellular vesicles/ml, preferably at least 1×10⁶, 1×10⁵, 1×10⁴, 1×10³, 1×10², 10 or 1 extracellular vesicles/ml. For the avoidance of doubt, it is to be understood that while the biomarker or EVs are detected at such concentrations, it is not essential to these embodiments that the concentration of the biomarker or EVs is determined. WO2022/122768, which is incorporated herein by reference, discloses a technique, termed O-NEXOS, for detecting EVs. In some embodiments, this technique is used to detect the presence of the biomarker at the required sensitivity. For example, the presence of the candidate biomarker can be detected using a reporter assay, such as horseradish peroxidase (HRP), a fluorophore conjugated to an antibody or E-NEXOS.

In some embodiments, the method further comprises determining the concentration of the biomarker in the sample. The concentration of the biomarker in the sample can be determined by quantifying the number of biomarkers in the sample using a reporter assay, such as horseradish peroxidase (HRP) or a fluorophore conjugated to an antibody via a calibration curve.

In some embodiments, the method applies to identifying a plurality of candidate biomarkers as a combination of biomarkers present at elevated levels in tissue of the defined category. The method comprises the steps of:
i. identifying a plurality of candidate biomarkers;
ii. providing a plurality of binding agents, each binding agent being capable of binding specifically to a respective candidate biomarker identified in step i;
iii. detecting the presence of each of the candidate biomarkers on an EV obtained from a sample of tissue of the defined category by binding the binding agents to the respective candidate biomarkers in the sample wherein EVs from tissue of the defined category preferentially bind a combination of the plurality of binding agents compared with EVs from the at least one different category of tissue from the organism; and
iv. selecting the plurality of candidate biomarkers as a combination of biomarkers present at elevated levels in tissue of the defined category.

In embodiments where the method comprises identifying a plurality of binding agents, a plurality of binding agents refers to at least 2 binding agents, at least 3 binding agents, or at least 4 binding agents. Preferably, a plurality of candidate biomarkers refers at least 4 binding agents.

In embodiments where the method comprises identifying a plurality of candidate biomarkers, a plurality of candidate biomarkers refers to at least 2 biomarkers, at least 3 biomarkers, or at least 4 biomarkers. Preferably, a plurality of candidate biomarkers refers to at least 4 biomarkers. Preferably, the number of binding agents corresponds to the number of biomarkers.

The advantage of identifying a plurality of candidate biomarkers present at elevated levels in tissue of the defined category is that, by providing the plurality of binding agents which are capable of binding specifically to the respective candidate biomarkers, it increases the number of EVs which can be obtained from a sample of tissue of the defined category, particularly in biofluids. As an example, the constituents secreted into blood is diverse across different individuals. Additionally, as discussed above, EVs are heterogeneous particles that are naturally secreted by cells in tissue in a dynamic fashion. Tissues also contain multiple types of cells. EVs secreted from cells of tissue of the defined category therefore may present different biomarkers present at elevated levels in the tissue of the defined category. Targeting a plurality of candidate biomarkers present at elevated levels in tissue of the defined category using a plurality of binding agents therefore allows for the detection of heterogeneous EVs secreted from cells of tissue of the defined category.

In some embodiments, the plurality of binding agents are provided sequentially. For example, a first binding agent is provided which is capable of binding specifically to the respective candidate biomarker on EVs, the EVs are then captured and isolated (as described below) using the first binding agent to generate an enriched fraction of EVs which present the first candidate biomarker. Subsequently a second binding agent is provided which is capable of binding specifically to the respective candidate biomarker on EVs in the enriched fraction of EVs and the EVs are captured and isolated using the second binding agent to generate an enriched fraction of EVs which present the first and second candidate biomarkers. In embodiments where the plurality of binding agents are provided sequentially, another advantage of identifying a plurality of candidate biomarkers is that EVs from a defined category of tissue may be identified more accurately. For example, in a scenario where there are first, second and third categories of tissue, there may be a first biomarker present on EVs from the first and second categories of tissue and a second biomarker present on EVs from the second and third categories of tissue. Therefore, if a panel of binding agents which bind to the first and second biomarkers is provided sequentially, this allows EVs from the second category of tissue to be captured and isolated from EVs from the first and third categories of tissue.

In some embodiments, the method further comprises contacting EVs from the at least one different category of tissue with the or each binding agent and detecting binding of the EVs from the at least one different category of tissue to the or each binding agent.

For example, in embodiments where the tissue of the defined category is liver tissue, the method further comprises contacting EVs from non-liver tissue (such as lung tissue, kidney tissue or breast tissue) with the or each binding agent, and detecting binding of the EVs from the non-liver tissue to the or each binding agent.

In some embodiments, the EVs from tissue of the defined category preferentially bind the or each binding agent compared with EVs from at least 2 different categories of tissue from the organism. For example, in embodiments where the tissue of the defined category is liver tissue, the EVs from liver tissue preferentially bind the or each binding agent compared with EVs from 2 non-liver tissues, such as kidney tissue and breast tissue, or from 3 non-liver tissues, such as lung tissue, kidney tissue or breast tissue.

In some embodiments, the EVs from tissue of the defined category preferentially bind the or each binding agent compared with EVs from all other categories of tissue from the organism.

In some embodiments, the method further comprises the step of detecting the presence of the or each candidate biomarker on an EV obtained from a cultured cell line which is representative of tissue of the defined category. In some embodiments, the extracellular vesicles obtained from a cultured cell line which is representative of tissue of the defined category preferentially bind the binding agent compared with extracellular vesicles obtained from a cultured cell line which is representative of at least one different category of tissue from the organism. In some embodiments where the tissue of the defined category is liver tissue, the cultured cell line is HepG2. In some embodiments, extracellular vesicles obtained from the HepG2 cell line preferentially bind the binding agent compared with extracellular vesicles obtained from a cultured cell line which is representative of non-liver tissue, for example a cultured cell line which is representative of breast tissue such as MCF-7 or BT-474.

In some embodiments, the EVs obtained from the cultured cell line is in plasma which has been spiked with the EVs.

In some embodiments, the EVs obtained from a sample of tissue is in plasma which has been spiked with the EVs after the EVs has been obtained from the sample of tissue.

In some embodiments, the sample of tissue of the present invention is healthy tissue or diseased tissue. Preferably, the healthy tissue is liver tissue. Preferably, the diseased tissue is a disease associated with liver tissue, for example liver cancer or HCC. In some embodiments the diseased tissue is cancer tissue. One reason for the sample of tissue being obtained from diseased tissue is that cells in certain diseased tissues are poorly differentiated and may not express typical healthy tissue markers. In some embodiments, the method is performed firstly on EVs obtained from a sample of healthy tissue of the defined category and then is repeated in respect of EVs obtained from a sample of diseased tissue of the same category. In this way, it is confirmed that the biomarker is characteristic of EVs from tissue of the defined category whether healthy or diseased.

### Further Steps of Identifying a Candidate Biomarker

In some embodiments, the step of identifying a candidate biomarker comprises the steps of:
1) identifying potential candidate biomarkers expressed in the defined category of tissue;
2) identifying potential candidate biomarkers which are present in the defined category of tissue at a higher level than in at least one different category of tissue from the organism;
3) identifying potential candidate biomarkers which are transmembrane or surface proteins;
4) identifying potential candidate biomarkers which are present in extracellular vesicles; or
5) a combination of steps 1) to 4) performed in any order, and thereby identifying a candidate biomarker.

In some embodiments, the step of identifying a candidate biomarker comprises the steps of:
1) identifying potential candidate biomarkers expressed in the defined category of tissue;
2) identifying potential candidate biomarkers from within the potential candidate biomarkers identified in step 1) which are present in the defined category of tissue at a higher level than in at least one different category of tissue from the organism;
3) identifying potential candidate biomarkers from within the potential candidate biomarkers identified in step 2) which are transmembrane or surface proteins; and
4) validating a candidate biomarker from within the potential candidate biomarkers identified in step 3) which is present in EVs or validating candidate biomarkers from within the potential candidate biomarkers identified in step 3) which are present in EVs.

In some embodiments, prior to validating the candidate biomarker, the step of identifying a candidate biomarker further comprises a step of identifying potential candidate biomarkers from within the potential candidate biomarkers identified in step 3) by further ranking the expression of potential candidate biomarkers which are present in the defined category of tissue at a higher level in comparison to expression in at least one different category of tissue from the organism (step 3b).

In some embodiments, steps 1) to 3), and optionally 3b), are performed using bioinformatics database search and selection. Examples of bioinformatics database include, but are not limited to, UniProt, ProteomicsDB and Human Proteome Map.

In some embodiments, information such as protein mRNA expression and protein expression profiles is used to identify potential candidate biomarkers which are expressed in the defined category of tissue and are present in the defined category of tissue at a higher level than in at least one different category of tissue from the organism.

For example, a potential candidate biomarker is identified in step 1) if it is known to be expressed in the defined category of tissue. A potential candidate biomarker identified in step 1) is then identified in step 2) if it is known to be expressed in the defined category of tissue at a high level and it is known to be expressed in at least one different category at a low or medium level. Preferably, step 2) comprises identifying potential candidate biomarkers from within the potential candidate biomarkers identified in step 1) which are present in the defined category of tissue at a higher level than all other different categories of tissue from the organism. For example, a potential candidate biomarker identified in step 1) is then identified in step 2) if it is known to be expressed in the defined category of tissue at a high level and it is known to be expressed in all other categories of tissue at a low or medium level.

In some embodiments, information such as subcellular location is used to identify potential candidate biomarkers which are transmembrane or surface proteins. In some embodiments, potential candidate biomarkers are identified in step 3) if they are known to be located in, or partially located in, the cell membrane.

In some embodiments, step 4) comprises evaluating the potential candidate biomarkers using a cell based assay, such as artificial plasma systems, in vitro cultured cell lines, and/or ex vivo tissue samples. For example, in some embodiments the validation step comprises evaluating the potential candidate biomarkers using cultured cell lines from the tissue of the defined category. EVs are secreted from the cultured cell lines. The potential candidate biomarkers pass the validation step if it is observed that the or each binding agent is capable of binding to the or each potential candidate biomarker on EVs secreted from the cultured cell lines from the tissue of the defined category at a desired threshold of sensitivity. The potential candidate biomarkers do not pass the validation step if it is observed that the or each binding agent is not capable of binding to the or each potential candidate biomarker on EVs secreted from the cultured cell lines from the tissue of the defined category at the desired threshold of sensitivity.

The advantage of performing steps 1) to 3) is that it limits the list of potential candidate biomarkers to those which are more likely to be present at elevated levels in tissue of a defined category in an organism. In particular, step 1) limits the list of potential candidate biomarkers to those expressed in the defined category of tissue. Step 2) limits the list of potential candidate biomarkers to those which are present in the defined category of tissue at a higher level than in at least one different category of tissue from the organism. Step 3) limits the list of potential candidate biomarkers to those which are transmembrane or surface proteins. Transmembrane or surface proteins are advantageous to the method of the present invention because they are exposed on the external side of the plasma membrane of an EV and are therefore accessible for binding by the or each binding agent without the need for permeabilising the EV plasma membrane.

The advantage of performing step 4) is that it confirms that the potential candidate biomarker is present on the surface of EVs and is accessible for binding by the binding agent. In vitro artificial plasma systems and cultured cell lines provide a simplified model of the in vivo environment. In contrast, ex vivo tissue samples are taken from the organism and therefore accurately reflect the in vivo environment.

In some embodiments, step 3) further comprises the step of fractionating a precursor sample of tissue of the defined category enriched in EVs in order to provide the sample of tissue of the defined category. For example, the precursor sample can be separated into fractions using size exclusion chromatography.

In some embodiments, the biomarker is specific for tissue of the defined category. That is to say the biomarker is present only in the target tissue, and it is not present in non-target tissue. In other embodiments, a combination of biomarkers is specific for tissue of the defined category.

While the above embodiments have been described comprising all of steps 1) to 4), it is to be understood that in alternative embodiments, only one of steps 1), 2), 3) or 4) is performed in order to select the candidate biomarker. In alternative embodiments, more than one of steps 1) to 4) is performed but in an alternative order from that set out above.

In embodiments where the defined category of tissue is liver tissue, it is preferred that the biomarker is ASGR1, ASGR2, TFR2, SLCO1B1, SLC38A3, TMEM56, UNC93A, SLC22A9, SLC2A2 or FXYD1, or a combination thereof. Most preferably, the biomarker is ASGR1, ASGR2, TFR2 or SLCO1B1, or a combination thereof.

As used herein, In preferred embodiments the biomarker has the amino acid sequence corresponding to the respective Uniprot ID number shown in any one of Tables 1 to 3.

In embodiments where the biomarker is ASGR1, the binding agent is preferably an anti-ASGR1 antibody. In embodiments where the biomarker is ASGR2, the binding agent is preferably an anti-ASGR2 antibody. In embodiments where the biomarker is TFR2, the binding agent is preferably an anti-TFR2 antibody. In embodiments where the biomarker is SLCO1B1, the binding agent is preferably an anti-SLCO1B1 antibody. In embodiments where the biomarker is SLC38A3, the binding agent is preferably an anti-SLC38A3 antibody. In embodiments where the biomarker is TMEM56, the binding agent is preferably an anti-TMEM56 antibody. In embodiments where the biomarker is UNC93A, the binding agent is preferably an anti-UNC93A antibody. In embodiments where the biomarker is SLC22A9, the binding agent is preferably an anti-SLC22A9 antibody. In embodiments where the biomarker is SLC2A2, the binding agent is preferably an anti-SLC2A2 antibody. In embodiments where the biomarker is FXYD1, the binding agent is preferably an anti-FXYD1 antibody.

In embodiments comprising a plurality of binding agents, each binding agent is preferably provided in a proportion of 10-50% of the total, wherein the total mixture of binding agents is equal to 100%.

In embodiments comprising a plurality of binding agents consisting of the anti-ASGR1 antibody, the anti-ASGR2 antibody, the anti-SLCO1B1 antibody and the anti-TFR2 antibody, the anti-ASGR1 antibody is preferably provided in a proportion of 30-50% of the total, and each the other antibodies are preferably provided in a proportion of 10-30% of the total, wherein the total mixture of binding agents is equal to 100%. In some embodiments, the anti-ASGR1 antibody is preferably provided in a proportion of 40% of the total, and each of the anti-ASGR2 antibody, the anti-SLCO1B1 antibody and the anti-TFR2 antibody is preferably provided in a proportion of 20% of the total of the four antibodies (this equates to a final ratio of 4:2:2:2).

It should be understood that, in some embodiments, the binding agents can be an antigen binding fragment of an antibody.

### A Method of Enriching a Biological Sample Comprising a Mixture of EVs

The present invention also provides a method of enriching a biological sample comprising a mixture of EVs comprising the steps of:
a) identifying one or more biomarkers present at elevated levels in a tissue, such as tissue of a defined category, by performing the method described above;
b) capturing EVs in the biological sample, wherein the extracellular vesicles display the or each biomarker identified in step a), to generate an enriched fraction of EVs from tissue of the defined category.

The present invention also provides a method of enriching a biological sample comprising a mixture of EVs comprising the steps of:
capturing EVs in the biological sample, wherein the EVs display one or more biomarkers, to generate an enriched fraction of EVs,
wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

In some embodiments, the EVs display two or more, three or more, or four or more biomarkers.

In some embodiments, the step of capturing extracellular vesicles comprises capturing extracellular vesicles based on the extracellular vesicles displaying one or more biomarkers and wherein the one or biomarkers consist of ASGR1, ASGR2, TFR2, and SLCO1B1.

In some embodiments, the or each biomarker is present at elevated levels in a tissue of a defined category. In some embodiments, the or each biomarker present at elevated levels in tissue of the defined category is identified using the method of identification disclosed herein.

In some embodiments, the EVs are captured using one or more binding agents which are capable of binding specifically to the or each biomarker displayed on EVs.

In some embodiments, the or each biomarker is ASGR1 and the or each binding agent is an anti-ASGR1 antibody or antigen binding fragment thereof, the or each biomarker is

ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is

SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is

ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

In some embodiments, the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene.

In embodiments comprising a plurality of binding agents, each binding agent is preferably provided in a proportion of 10-50% of the total, wherein the total mixture of binding agents is equal to 100%.

In embodiments comprising a plurality of binding agents consisting of the anti-ASGR1 antibody, the anti-ASGR2 antibody, the anti-SLCO1B1 antibody and the anti-TFR2 antibody, the anti-ASGR1 antibody is preferably provided in a proportion of 30-50% of the total, and each the other antibodies are preferably provided in a proportion of 10-30% of the total, wherein the total mixture of binding agents is equal to 100%. In some embodiments, the anti-ASGR1 antibody is preferably provided in a proportion of 40% of the total, and each of the anti-ASGR2 antibody, the anti-SLCO1B1 antibody and the anti-TFR2 antibody is preferably provided in a proportion of 20% of the total of the four antibodies (this equates to a final ratio of 4:2:2:2).

It should be understood that, in some embodiments, the binding agents can be an antigen binding fragment of an antibody.

It is preferred that the biological sample is a sample of tissue or a biofluid. In embodiments where the biological sample is a biofluid, the biofluid comprises circulating EVs. Preferably the biofluid is blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

Most preferably, the biofluid is blood, urine, cerebrospinal fluid or mucus.

In some embodiments, the mixture of EVs comprises extracellular vesicles from tissue of the defined category and EVs from at least one different category of tissue from the organism. For example, in embodiments where the tissue of the defined category is liver tissue, the mixture of EVs, preferably where the biological sample is a biofluid, may comprise EVs from liver tissue and EVs from at least one other category of tissue such as breast tissue.

In some embodiments, the step of capturing EVs in the biological sample further comprises isolating the extracellular vesicles displaying the or each biomarker identified in the step or method of identifying a biomarker disclosed herein which are captured using the or each biomarker.

In some embodiments, the isolated extracellular vesicles are intact extracellular vesicles.

This permits further downstream processing of intact extracellular vesicles such as determining the presence of other biomarkers on or in the extracellular vesicles.

In some embodiments, the method of enriching a biological sample further comprises the step of releasing the contents of the captured or isolated extracellular vesicles. For example, the contents of the isolated extracellular vesicles can be released by lysis of the EV membrane. This permits further downstream processing of the aggregated contents of the captured or isolated extracellular vesicles.

In some embodiments, the step of capturing EVs in the biological sample comprises using the or each binding agent. For example, in some embodiments, the binding agent is provided on a substrate (e.g. a magnetic bead) and the biological sample is contacted with the substrate. The binding agent binds to extracellular vesicles presenting the biomarker thereby “capturing” the extracellular vesicles. The unbound biological sample is then washed away, leaving the captured extracellular vesicles bound to the substrate via the binding agent. In some particular embodiments, the captured extracellular vesicles are then released from the binding agent in order to provide an enriched fraction of extracellular vesicles or fully isolated extracellular vesicles.

The advantage of performing the method of enriching a biological sample comprising a mixture of EVs is that an enriched fraction of EVs which are present at elevated levels in a tissue of a defined category is generated. For example, in embodiments where the tissue of the defined category is liver tissue, the method is capable of generating a fraction of EVs which are present at elevated levels in liver tissue from a mixture of EVs, such as EVs from a non-liver tissue from the organism.

It should be understood that, once the or each candidate biomarker is identified using the method of identification disclosed herein, the biomarker(s) may be used without performing the identification step in the method of enriching a biological sample comprising a mixture of EVs.

### A Method of Analysing a Biological Sample Comprising a Mixture of EVs

The present invention also provides a method of analysing a biological sample comprising a mixture of EVs comprising the steps of:
a) identifying one or more biomarkers present at elevated levels in a tissue, such as tissue of a defined category, by performing the method of identifying a biomarker present at elevated levels in tissue of a defined category in an organism of the claimed invention;
b) capturing EVs in the biological sample, wherein the extracellular vesicles display the or each biomarker identified in step a), to generate an enriched fraction of EVs; and
c) analysing the contents of an EV in the enriched fraction obtained in step b).

The present invention also provides a method of analysing a biological sample comprising a mixture of EVs comprising the steps of:
a) capturing EVs in the biological sample, wherein the EVs display one or more biomarkers, to generate an enriched fraction of EVs; and
b) analysing the contents of an EV in the enriched fraction obtained in step a), wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

In some embodiments, the EVs display two or more, three or more, or four or more biomarkers.

In some embodiments, the step of capturing extracellular vesicles comprises capturing extracellular vesicles based on the extracellular vesicles displaying one or more biomarkers and wherein the one or biomarkers consist of ASGR1, ASGR2, TFR2, and SLCO1B1.

In some embodiments, the or each biomarker is present at elevated levels in a tissue of a defined category. In some embodiments, the or each biomarker present at elevated levels in tissue of the defined category is identified using the method of identification disclosed herein.

In some embodiments, the EVs are captured using one or more binding agents which are capable of binding specifically to the or each biomarker displayed on EVs.

In some embodiments, the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

In some embodiments, the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene.

In embodiments comprising a plurality of binding agents, each binding agent is preferably provided in a proportion of 10-50% of the total, wherein the total mixture of binding agents is equal to 100%.

In embodiments comprising a plurality of binding agents consisting of the anti-ASGR1 antibody, the anti-ASGR2 antibody, the anti-SLCO1B1 antibody and the anti-TFR2 antibody, the anti-ASGR1 antibody is preferably provided in a proportion of 30-50% of the total, and each the other antibodies are preferably provided in a proportion of 10-30% of the total, wherein the total mixture of binding agents is equal to 100%. In some embodiments, the anti-ASGR1 antibody is preferably provided in a proportion of 40% of the total, and each of the anti-ASGR2 antibody, the anti-SLCO1B1 antibody and the anti-TFR2 antibody is preferably provided in a proportion of 20% of the total of the four antibodies (this equates to a final ratio of 4:2:2:2).

It should be understood that, in some embodiments, the binding agents can be an antigen binding fragment of an antibody.

It is preferred that the biological sample is a sample of tissue or a biofluid. In embodiments where the biological sample is a biofluid, the biofluid comprises circulating

EVs. Preferably the biofluid is blood, urine, saliva, lymph, bile, cerebrospinal fluid, phlegm, mucus, tears, Bronchoalveolar Lavage (BAL) fluid, earwax, sweat faeces, breast milk, interstitial fluids, vaginal fluids, semen, gastric juice, blister fluid or cyst fluid. Most preferably, the biofluid is blood, urine, cerebrospinal fluid or mucus.

In some embodiments, the mixture of EVs comprises EVs from tissue of the defined category and EVs from at least one different category of tissue from the organism. For example, in embodiments where the tissue of the defined category is liver tissue, the mixture of EVs, preferably where the biological sample is a biofluid, may comprise EVs from liver tissue and EVs from at least one other category of tissue such as breast tissue.

In some embodiments, the step of capturing EVs in the biological sample further comprises isolating the EVs displaying the or each biomarker identified in the step or method of identifying a biomarker disclosed herein which are captured using the or each biomarker.

In some embodiments, the step of capturing EVs in the biological sample comprises using the or each binding agent.

Examples of the contents of an EV include the protein, DNA, RNA, lipid, post-translational modifications or metabolite content of an extracellular vesicle. By performing the method of analysing a biological sample, the contents of an extracellular vesicle, such as protein, DNA, RNA, lipid, post-translational modifications or metabolite content, can be present at elevated levels in tissue of a defined category in an organism. Furthermore, where the biological sample is obtained from an individual of a particular demographic group, risk group or patient group, an association can be made between the contents of EVs and individuals in the studied group. For example, if the biological sample is obtained from a liver cancer patient and the defined category of tissue is liver tissue, then it is possible to use the contents of the EV to search for and identify biological markers for liver cancer.

In some embodiments, the step of analysing the contents of an EV comprises the lysis of an EV to release its contents.

In some embodiments, the contents of an EV are analysed by mass spectrometry and/or by RNA sequencing.

In some embodiments, the EVs are eluted intact for downstream applications requiring intact EVs as for example in biomarker quantification or EV therapies given the immunomodulatory and anti-inflammatory properties of EVs obtained from certain tissues (e.g. umbilical cord, bone marrow, adipose tissue) (Zheng et al., 2021; Weng et al., 2021).

It should be understood that, once the or each candidate biomarker is identified using the method of identification disclosed herein, the biomarker(s) may be used without performing the identification step in further methods, in particular in the method of analysing a biological sample comprising a mixture of EVs.

### Use of One or More Biomarkers, or One or More Binding Agents, to Generate an Enriched Fraction of EVs

The present invention also provides a use of one or more biomarkers displayed on EVs to generate an enriched fraction of EVs, wherein the enriched fraction of EVs are secreted from tissue of a defined category in an organism, wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

In some embodiments, two or more, three or more, or four or more biomarkers are used to generate the enriched fraction of EVs.

In some embodiments, the biomarkers consist of ASGR1, ASGR2, TFR2, and SLCO1B1.

In some embodiments, the or each biomarker is present at elevated levels in a tissue of the defined category. In some embodiments, the or each biomarker present at elevated levels in tissue of the defined category is identified using the method of identification disclosed herein

The present invention also provides a use of one or more binding agents to generate an enriched fraction of EVs, wherein the enriched fraction of EVs are secreted from tissue of a defined category in an organism, wherein the or each binding agent is capable of binding specifically to a biomarker displayed on EVs, wherein the or each binding agent is selected from the group consisting of an anti-ASGR1 antibody or antigen binding fragment thereof, an anti-ASGR2 antibody or antigen binding fragment thereof, an anti-TFR2 antibody or antigen binding fragment thereof, an anti-SLCO1B1 antibody or antigen binding fragment thereof, an anti-SLC38A3 antibody or antigen binding fragment thereof, an anti-TMEM56 antibody or antigen binding fragment thereof, an anti-UNC93A antibody or antigen binding fragment thereof, an anti-SLC22A9 antibody or antigen binding fragment thereof, an anti-SLC2A2 antibody or antigen binding fragment thereof, and an anti-FXYD1 antibody or antigen binding fragment thereof, or a combination thereof, preferably an anti-ASGR1 antibody or antigen binding fragment thereof, an anti-ASGR2 antibody or antigen binding fragment thereof, an anti-TFR2 antibody or antigen binding fragment thereof, or an anti-SLCO1B1 antibody or antigen binding fragment thereof, or a combination thereof.

In some embodiments, two or more, three or more, or four or more binding agents are used to generate the enriched fraction of EVs.

In some embodiments, the or each binding agent consists of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof. In preferred embodiments, the plurality of binding agents consists of the anti-ASGR1 antibody, the anti-ASGR2 antibody, the anti-SLCO1B1 antibody and the anti-TFR2 antibody.

It should be understood that the or each binding agent is capable of binding specifically to a respective biomarker displayed on EVs. In some embodiments, the or each biomarker is present at elevated levels in the tissue of the defined category. In some embodiments, the or each biomarker present at elevated levels in tissue of the defined category is identified using the method of identification disclosed herein.

In some embodiments, the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene.

In embodiments comprising a plurality of binding agents, each binding agent is preferably provided in a proportion of 10-50% of the total, wherein the total mixture of binding agents is equal to 100%.

In embodiments comprising a plurality of binding agents consisting of the anti-ASGR1 antibody, the anti-ASGR2 antibody, the anti-SLCO1B1 antibody and the anti-TFR2 antibody, the anti-ASGR1 antibody is preferably provided in a proportion of 30-50% of the total, and each the other antibodies are preferably provided in a proportion of 10-30% of the total, wherein the total mixture of binding agents is equal to 100%. In some embodiments, the anti-ASGR1 antibody is preferably provided in a proportion of 40% of the total, and each of the anti-ASGR2 antibody, the anti-SLCO1B1 antibody and the anti-TFR2 antibody is preferably provided in a proportion of 20% of the total of the four antibodies (this equates to a final ratio of 4:2:2:2).

It should be understood that, in some embodiments, the binding agents can be an antigen binding fragment of an antibody.

In some embodiments, the enriched fraction of EVs is enriched from a biological sample comprising a mixture of EVs. In preferred embodiments, the mixture of EVs comprises EVs from tissue of the defined category and EVs from at least one different category of tissue from the organism. For example, in embodiments where the tissue of the defined category is liver tissue, the mixture of EVs, preferably where the biological sample is a biofluid, may comprise EVs from liver tissue and EVs from at least one other category of tissue such as breast tissue.

It is preferred that the biological sample is a sample of tissue or a biofluid. In embodiments where the biological sample is a biofluid, the biofluid comprises circulating EVs. Preferably the biofluid is blood, urine, saliva, lymph, bile, cerebrospinal fluid, phlegm, mucus, tears, Bronchoalveolar Lavage (BAL) fluid, earwax, sweat faeces, breast milk, interstitial fluids, vaginal fluids, semen, gastric juice, blister fluid or cyst fluid. Most preferably, the biofluid is blood, urine, cerebrospinal fluid or mucus.

It is preferred that the defined category of tissue is liver tissue. In embodiments where the defined category of tissue is liver tissue, preferably the cells from which the EVs are secreted are hepatocytes, Kupffer cells, stellate cells or liver-resident dendritic cells. Most preferably, the cells from which the EVs are secreted are hepatocytes.

### A Method of Detecting One or More Biomarkers on or in EVs

The present invention also provides a method of detecting one or more biomarkers on or in EVs secreted from a tissue of a defined category, wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

In some embodiments, two or more, three or more, or four or more biomarkers are detected on or in EVs secreted from a tissue of a defined category.

In some embodiments, the biomarkers consist of ASGR1, ASGR2, TFR2, and SLCO1B1.

In some embodiments, the or each biomarker is present at elevated levels in the tissue of the defined category. In some embodiments, the or each biomarker present at elevated levels in tissue of the defined category is identified using the method of identification disclosed herein.

In some embodiments, the or each biomarker is detected by the binding of one or more binding agents, wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is

TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

In embodiments comprising the detection of two or more biomarkers, the biomarkers are detected by the binding of two or more binding agents. In embodiments comprising the detection of three or more biomarkers, the biomarkers are detected by the binding of three or more binding agents. In embodiments comprising the detection of four or more biomarkers, the biomarkers are detected by the binding of four or more binding agents.

In some embodiments, the or each binding agent consists of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof. In preferred embodiments, the plurality of binding agents consists of the anti-ASGR1 antibody, the anti-ASGR2 antibody, the anti-SLCO1B1 antibody and the anti-TFR2 antibody.

It should be understood that the or each binding agent is capable of binding specifically to a respective biomarker displayed on EVs. In some embodiments, the or each biomarker is present at elevated levels in the tissue of the defined category. In some embodiments, the or each biomarker present at elevated levels in tissue of the defined category is identified using the method of identification disclosed herein.

In some embodiments, the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene.

In embodiments comprising a plurality of binding agents, each binding agent is preferably provided in a proportion of 10-50% of the total, wherein the total mixture of binding agents is equal to 100%.

In embodiments comprising a plurality of binding agents consisting of the anti-ASGR1 antibody, the anti-ASGR2 antibody, the anti-SLCO1B1 antibody and the anti-TFR2 antibody, the anti-ASGR1 antibody is preferably provided in a proportion of 30-50% of the total, and each the other antibodies are preferably provided in a proportion of 10-30% of the total, wherein the total mixture of binding agents is equal to 100%. In some embodiments, the anti-ASGR1 antibody is preferably provided in a proportion of 40% of the total, and each of the anti-ASGR2 antibody, the anti-SLCO1B1 antibody and the anti-TFR2 antibody is preferably provided in a proportion of 20% of the total of the four antibodies (this equates to a final ratio of 4:2:2:2).

It should be understood that, in some embodiments, the binding agents can be an antigen binding fragment of an antibody.

In some embodiments, the or each biomarker is detected on or in EVs secreted from a tissue of a defined category in a biological sample comprising a mixture of EVs.

In preferred embodiments, the mixture of EVs comprises EVs from tissue of the defined category and EVs from at least one different category of tissue from the organism. For example, in embodiments where the tissue of the defined category is liver tissue, the mixture of EVs, preferably where the biological sample is a biofluid, may comprise EVs from liver tissue and EVs from at least one other category of tissue such as breast tissue.

It is preferred that the biological sample is a sample of tissue or a biofluid. In embodiments where the biological sample is a biofluid, the biofluid comprises circulating EVs. Preferably the biofluid is blood, urine, saliva, lymph, bile, cerebrospinal fluid, phlegm, mucus, tears, Bronchoalveolar Lavage (BAL) fluid, earwax, sweat faeces, breast milk, interstitial fluids, vaginal fluids, semen, gastric juice, blister fluid or cyst fluid. Most preferably, the biofluid is blood, urine, cerebrospinal fluid or mucus.

It is preferred that the defined category of tissue is liver tissue. In embodiments where the defined category of tissue is liver tissue, preferably the cells from which the EVs are secreted are hepatocytes, Kupffer cells, stellate cells or liver-resident dendritic cells. Most preferably, the cells from which the EVs are secreted are hepatocytes.

### A Kit for Enriching a Biological Sample Comprising a Mixture of EVs to Generate an Enriched Fraction of EVs

The present invention also provides a kit for enriching a biological sample comprising a mixture of EVs to generate an enriched fraction of EVs, wherein the enriched fraction of EVs are secreted from tissue of a defined category in an organism, wherein the kit comprises one or more binding agents which are capable of binding specifically to a biomarker present at elevated levels in tissue of the defined category, and wherein the or each binding agent is selected from the group consisting of an anti-ASGR1 antibody or antigen binding fragment thereof, an anti-ASGR2 antibody or antigen binding fragment thereof, an anti-TFR2 antibody or antigen binding fragment thereof, an anti-SLCO1B1 antibody or antigen binding fragment thereof, an anti-SLC38A3 antibody or antigen binding fragment thereof, an anti-TMEM56 antibody or antigen binding fragment thereof, an anti-UNC93A antibody or antigen binding fragment thereof, an anti-SLC22A9 antibody or antigen binding fragment thereof, an anti-SLC2A2 antibody or antigen binding fragment thereof, and an anti-FXYD1 antibody or antigen binding fragment thereof, or a combination thereof, preferably an anti-ASGR1 antibody or antigen binding fragment thereof, an anti-ASGR2 antibody or antigen binding fragment thereof, an anti-TFR2 antibody or antigen binding fragment thereof, or an anti-SLCO1B1 antibody or antigen binding fragment thereof, or a combination thereof.

In some embodiments, the kit comprises two or more, three or more, or four or more binding agents which are capable of binding specifically to a biomarker present at elevated levels in tissue of the defined category.

In some embodiments, the or each binding agent consists of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof. In preferred embodiments, the plurality of binding agents consists of the anti-ASGR1 antibody, the anti-ASGR2 antibody, the anti-SLCO1B1 antibody and the anti-TFR2 antibody.

It should be understood that the or each binding agent comprised in the kit is capable of binding specifically to a respective biomarker displayed on EVs. In some embodiments, the or each biomarker is present at elevated levels in the tissue of the defined category. In some embodiments, the or each biomarker present at elevated levels in tissue of the defined category is identified using the method of identification disclosed herein.

In some embodiments, the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene.

In embodiments comprising a plurality of binding agents, each binding agent is preferably provided in a proportion of 10-50% of the total, wherein the total mixture of binding agents is equal to 100%.

In embodiments comprising a plurality of binding agents consisting of the anti-ASGR1 antibody, the anti-ASGR2 antibody, the anti-SLCO1B1 antibody and the anti-TFR2 antibody, the anti-ASGR1 antibody is preferably provided in a proportion of 30-50% of the total, and each the other antibodies are preferably provided in a proportion of 10-30% of the total, wherein the total mixture of binding agents is equal to 100%. In some embodiments, the anti-ASGR1 antibody is preferably provided in a proportion of 40% of the total, and each of the anti-ASGR2 antibody, the anti-SLCO1B1 antibody and the anti-TFR2 antibody is preferably provided in a proportion of 20% of the total of the four antibodies (this equates to a final ratio of 4:2:2:2).

It should be understood that, in some embodiments, the binding agents can be an antigen binding fragment of an antibody.

In preferred embodiments, the mixture of EVs comprises EVs from tissue of the defined category and EVs from at least one different category of tissue from the organism. For example, in embodiments where the tissue of the defined category is liver tissue, the mixture of EVs, preferably where the biological sample is a biofluid, may comprise EVs from liver tissue and EVs from at least one other category of tissue such as breast tissue.

It is preferred that the biological sample is a sample of tissue or a biofluid. In embodiments where the biological sample is a biofluid, the biofluid comprises circulating EVs. Preferably the biofluid is blood, urine, saliva, lymph, bile, cerebrospinal fluid, phlegm, mucus, tears, Bronchoalveolar Lavage (BAL) fluid, earwax, sweat faeces, breast milk, interstitial fluids, vaginal fluids, semen, gastric juice, blister fluid or cyst fluid. Most preferably, the biofluid is blood, urine, cerebrospinal fluid or mucus.

It is preferred that the defined category of tissue is liver tissue. In embodiments where the defined category of tissue is liver tissue, preferably the cells from which the EVs are secreted are hepatocytes, Kupffer cells, stellate cells or liver-resident dendritic cells. Most preferably, the cells from which the EVs are secreted are hepatocytes.

The present invention also provides a method of manufacturing the kit disclosed herein, wherein the method comprises colocating the or each binding agent.

### An Enriched Fraction of EVs

The present invention also provides an enriched fraction of EVs, the fraction being enriched with EVs secreted from tissue of a defined category in an organism, which EVs display one or more biomarkers selected from the group consisting of ASGR1, ASGR2,

TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

In some embodiments, the EVs secreted from tissue of a defined category in an organism display two or more, three or more, or four or more biomarkers.

In some embodiments, the EVs secreted from tissue of a defined category in an organism display a plurality of biomarkers comprising ASGR1, ASGR2, TFR2, and SLCO1B1.

In some embodiments, the or each biomarker is present at elevated levels in tissue of the defined category.

It is preferred that the defined category of tissue is liver tissue. In embodiments where the defined category of tissue is liver tissue, preferably the cells from which the EVs are secreted are hepatocytes, Kupffer cells, stellate cells or liver-resident dendritic cells. Most preferably, the cells from which the EVs are secreted are hepatocytes.

In some embodiments, the enriched fraction of EVs is enriched from a biological sample comprising a mixture of EVs.

It is preferred that the biological sample is a sample of tissue or a biofluid. In embodiments where the biological sample is a biofluid, the biofluid comprises circulating

EVs. Preferably the biofluid is blood, urine, saliva, lymph, bile, cerebrospinal fluid, phlegm, mucus, tears, Bronchoalveolar Lavage (BAL) fluid, earwax, sweat faeces, breast milk, interstitial fluids, vaginal fluids, semen, gastric juice, blister fluid or cyst fluid. Most preferably, the biofluid is blood, urine, cerebrospinal fluid or mucus.

In some embodiments, the enriched fraction of EVs is generated by performing the methods of enriching a biological sample comprising a mixture of EVs described in detail above, or following use of one or more biomarkers or one or more binding agents described in detail above.

In some embodiments, the enriched fraction of EVs is generated and the contents of the EVs subsequently analysed by performing the method of analysing a biological sample described in detail above.

### Specific Embodiment

In a specific embodiment, the present invention provides a method of identifying a biomarker present at elevated levels in liver tissue in an individual in comparison to the level in at least one of lung, kidney or breast tissue. The biomarker is detectable on the surface of EVs secreted from cells of liver tissue. The method comprises the steps of:
i. identifying a candidate biomarker;
ii. providing an antibody which is capable of binding specifically to the candidate biomarker identified in step i.;
iii. detecting the presence of the candidate biomarker on an EV obtained from a sample of liver tissue from the individual by binding the antibody to the candidate biomarker in the sample wherein EVs from liver tissue preferentially bind the antibody compared with EVs from at least one of lung, kidney or breast tissue from the individual; and
iv. selecting the candidate biomarker as a biomarker present at elevated levels in liver tissue.

In some embodiments, the method applies to identifying a plurality of candidate biomarkers as a combination of biomarkers present at elevated levels in liver tissue in comparison to the level in at least one of lung, kidney or breast tissue. The method comprises the steps of:
i. identifying four candidate biomarkers;
ii. providing four antibodies, each antibody being capable of binding specifically to a respective candidate biomarker identified in step i;
iii. detecting the presence of each of the candidate biomarkers on an EV obtained from a sample of liver tissue from the individual by binding the antibodies to the respective candidate biomarkers in the sample wherein EVs from liver tissue preferentially bind a combination of the plurality of antibodies compared with EVs from at least one of lung, kidney or breast tissue from the organism; and
iv. selecting the four candidate biomarkers as a combination of biomarkers present at elevated levels in liver tissue.

In some embodiments, the four antibodies are provided in a mixture of equal proportions. In other embodiments, the four antibodies are provided in a mixture of unequal proportions, such as the four antibodies are provided in a mixture at a final ratio of 4:2:2:2.

In some embodiments, the or each antibody is provided attached to a magnetic bead.

One embodiment comprises a method of enriching a blood sample taken from an individual comprising a mixture of EVs comprising the steps of:
a) identifying a biomarker present at elevated levels in liver tissue by performing the method described above;
b) capturing EVs in the blood sample, wherein the EVs display the or each biomarker identified in step a), to generate an enriched fraction of EVs which derive from liver tissue.

One embodiment comprises a method of enriching a blood sample taken from an individual comprising a mixture of EVs comprising the step of:
capturing EVs in the blood sample, wherein the EVs display one or more biomarkers present at elevated levels in liver tissue, to generate an enriched fraction of EVs which derive from liver tissue,
wherein the or each biomarker is selected from the group consisting of ASGR1, ASGR2, TFR2, SLCO1B1, SLC38A3, TMEM56, UNC93A, SLC22A9, SLC2A2, and FXYD1.

In some embodiments, the step of capturing EVs in the blood sample displaying the or each biomarker further comprises isolating the EVs the captured extracellular vesicles which derive from liver tissue using the or each biomarker.

One embodiment comprises a method of analysing a blood sample taken from an individual comprising a mixture of EVs comprising the steps of:
a) identifying a biomarker present at elevated levels in liver tissue by performing the method of identifying a biomarker present at elevated levels in liver tissue in an organism of the claimed invention;
b) capturing EVs in the blood sample, wherein the EVs display the or each biomarker identified in step a), to generate an enriched fraction of EVs which derive from liver tissue; and
c) analysing the contents of an EV in the enriched fraction obtained in step b).

One embodiment comprises a method of analysing a blood sample taken from an individual comprising a mixture of EVs comprising the steps of:
a) capturing EVs in the blood sample, wherein the EVs display one or more biomarkers present at elevated levels in liver tissue, to generate an enriched fraction of EVs which derive from liver tissue; and
b) analysing the contents of an EV in the enriched fraction obtained in step a), wherein the or each biomarker is selected from the group consisting of ASGR1, ASGR2, TFR2, SLCO1B1, SLC38A3, TMEM56, UNC93A, SLC22A9, SLC2A2, and FXYD1.

In some embodiments, the step of capturing EVs in the blood sample comprises using the or each antibody, such that the antibody is capable of binding specifically to a biomarker.

One embodiment comprises a use of one or more biomarkers to generate an enriched fraction of EVs from a blood sample taken from an individual comprising a mixture of EVs, wherein the enriched fraction of EVs is secreted from cells of liver tissue, wherein the or each biomarker is selected from the group consisting of ASGR1, ASGR2, TFR2, SLCO1B1, SLC38A3, TMEM56, UNC93A, SLC22A9, SLC2A2, and FXYD1.

One embodiment comprises a use of one or more antibodies to generate an enriched fraction of EVs from a blood sample taken from an individual comprising a mixture of EVs, wherein the enriched fraction of EVs is secreted from cells of liver tissue, wherein the or each antibody is selected from the group consisting of an anti-ASGR1 antibody, an anti-ASGR2 antibody, an anti-TFR2 antibody, an anti-SLCO1B1 antibody, an anti-SLC38A3 antibody, an anti-TMEM56 antibody, an anti-UNC93A antibody, an anti-SLC22A9 antibody, an anti-SLC2A2 antibody, and an anti-FXYD1 antibody.

In some embodiments, the use comprises a plurality of antibodies consisting of the anti-ASGR1 antibody, the anti-ASGR2 antibody, the anti-SLCO1B1 antibody and the anti-TFR2 antibody.

One embodiment comprises a method of detecting one or more biomarkers on or in EV secreted from cells of liver tissue, wherein the or each biomarker is selected from the group consisting of ASGR1, ASGR2, TFR2, SLCO1B1, SLC38A3, TMEM56, UNC93A, SLC22A9, SLC2A2, and FXYD1.

One embodiment comprises a kit for enriching a blood sample taken from an individual comprising a mixture of EVs to generate an enriched fraction of EVs, wherein the enriched fraction of EVs is secreted from cells of liver tissue, wherein the kit comprises one or more antibodies which are capable of binding specifically to a biomarker present at elevated levels in liver tissue, and wherein the or each antibody is selected from the group consisting of an anti-ASGR1 antibody, an anti-ASGR2 antibody, an anti-TFR2 antibody, an anti-SLCO1B1 antibody, an anti-SLC38A3 antibody, an anti-TMEM56 antibody, an anti-UNC93A antibody, an anti-SLC22A9 antibody, an anti-SLC2A2 antibody, and an anti-FXYD1 antibody.

In some embodiments, the kit comprises a plurality of antibodies consisting of the anti-ASGR1 antibody, the anti-ASGR2 antibody, the anti-SLCO1B1 antibody and the anti-TFR2 antibody.

One embodiment comprises a method of manufacturing a kit for enriching a blood sample taken from an individual comprising a mixture of EVs to generate an enriched fraction of EVs, wherein the enriched fraction of EVs is secreted from cells of liver tissue, wherein the kit comprises one or more antibodies which are capable of binding specifically to a biomarker present at elevated levels in liver tissue, wherein the or each antibody is selected from the group consisting of an anti-ASGR1 antibody, an anti-ASGR2 antibody, an anti-TFR2 antibody, an anti-SLCO1B1 antibody, an anti-SLC38A3 antibody, an anti-TMEM56 antibody, an anti-UNC93A antibody, an anti-SLC22A9 antibody, an anti-SLC2A2 antibody, and an anti-FXYD1 antibody, and wherein the method comprises colocating the or each antibody,

One embodiment comprises an enriched fraction of EVs which are derived from liver tissue, the fraction being enriched with EVs secreted from cells of liver tissue, which EVs display one or more biomarkers selected from the group consisting of ASGR1, ASGR2, TFR2, SLCO1B1, SLC38A3, TMEM56, UNC93A, SLC22A9, SLC2A2, and FXYD1.

In some embodiments, the enriched fraction of EVs is enriched from a blood sample taken from an individual comprising a mixture of EVs.

In some variants of the embodiments described above, the or each biomarker consists of ASGR1, ASGR2, TFR2, and SLCO1B1.

In some variants of the embodiments described above, the or each biomarker is ASGR1 and the or each antibody is an anti-ASGR1 antibody, the or each biomarker is ASGR2 and the or each antibody is an anti-ASGR2 antibody, the or each biomarker is TFR2 and the or each antibody is an anti-TFR2 antibody, or the or each biomarker is SLCO1B1 and the or each antibody is an anti-SLCO1B1 antibody.

In some variants of the embodiments described above, the four antibodies are provided in a mixture of equal proportions. In other embodiments, the four antibodies are provided in a mixture of unequal proportions, such as the four antibodies are provided in a mixture at a final ratio of 4:2:2:2.

In some variants of the embodiments described above, the or each antibody is provided attached to a magnetic bead.

In some variants of the embodiments described above, the mixture of EVs comprises EVs from liver tissue, and EVs from lung, kidney or breast tissue.

### EXAMPLES

Hereinafter, the invention will be specifically described with reference to the Examples.

### EXAMPLE 1: IDENTIFICATION OF BIOMARKERS OF LIVER TISSUE

### 1-1) Identification of Potential Candidate Biomarkers of Liver Tissue

Liver-specific biomarkers were identified using bioinformatics database search and selection.

mRNA expression and protein expression profiles from The Human Proteins Atlas (latest accessed on 11/2021) database were used to retrieve and obtain an initial ranking of hepatocytes specific markers.

Four combinations of search terms were used to obtain four different list of markers which were then merged resulting in a total of 412 candidate biomarkers:
1) proteins that were enriched or enhanced at the transcript level for liver and were not annotated as having high or medium protein expression for other tissues;
2) proteins that were considered to be highly expressed with high specificity for hepatocytes, with protein expression in hepatocytes annotated as high, and were not annotated as having high or medium protein expression for other tissues besides liver;
3) proteins that were considered to be highly expressed with specificity for hepatocytes, with protein expression in hepatocytes annotated as high, and were not annotated as having high protein expression for other tissues besides liver;
4) proteins that were considered to be medium expressed with high specificity for hepatocytes, with protein expression in hepatocytes annotated as medium, and were not annotated as having high or medium protein expression for other tissues besides liver.

UniProt was used to retrieve the subcellular location for these proteins. Proteins annotated as located in the membrane were selected for the next steps. A total of 88 candidate biomarkers were taken forward (Table 1).

TABLE 1
Top 88 candidate biomarkers

| | | | | Human | (Human | |
|---|---|---|---|---|---|---|
| | | Human | Human | Protein | Protein | |
| | | Protein | Protein | Atlas | Atlas) | |
| | | Atlas | Atlas | (mRNA | mRNA only | |
| Gene | Uniprot | (Protein | (Protein | overexpressed | expressed | Human |
| Name | ID | expression) | specificity) | in liver) | in liver | proteome |
| PLPPR2 | Q96GM1 | High | Medium | No | No | NA |
| SLC2A2 | P11168 | High | Medium | No | No | Medium |
| ABCB11 | O95342 | High | High | Yes | No | High |
| ABCB4 | P21439 | High | High | Yes | No | High |
| ASGR1 | P07306 | High | High | Yes | No | High |
| SLCO1B3 | Q9NPD5 | High | High | Yes | No | High |
| TFR2 | Q9UP52 | High | High | Yes | No | High |
| SLC22A7 | Q9Y694 | High | Medium | No | No | High |
| GHR | P10912 | High | Medium | No | No | Absent |
| RNF130 | Q86XS8 | High | Medium | No | No | Absent |
| SLC17A4 | Q9Y2C5 | High | Medium | No | No | Absent |
| SLC13A5 | Q86YT5 | Low | High | Yes | No | High |
| AQP9 | O43315 | Medium | High | Yes | No | High |
| ASGR2 | P07307 | Medium | High | Yes | No | High |
| SLC10A1 | Q14973 | Medium | High | Yes | No | High |
| SLCO1B1 | Q9Y6L6 | Medium | High | Yes | Yes | High |
| ADGRG7 | Q96K78 | | | Yes | No | NA |
| SLC38A3 | Q99624 | | | Yes | No | Medium |
| FXYD1 | O00168 | | | Yes | No | Medium |
| TMPRSS9 | Q7Z410 | | | Yes | No | Medium |
| SLC22A9 | Q8IVM8 | | | Yes | No | Low |
| PTP4A1 | Q93096 | | | Yes | No | Low |
| SLC38A4 | Q969I6 | | | Yes | No | High |
| CLEC4G | Q6UXB4 | | | Yes | No | High |
| TMEM82 | A0PJX8 | | | Yes | No | High |
| ELFN1 | P0C7U0 | | | Yes | No | High |
| SLC7A2 | P52569 | | | Yes | No | High |
| TMEM56 | Q96MV1 | | | Yes | No | High |
| RTP3 | Q9BQQ7 | | | Yes | Yes | High |
| SLC17A2 | O00624 | | | Yes | Yes | High |
| DNAJC22 | Q8N4W6 | | | Yes | No | Absent |
| LRRC3 | Q9BY71 | | | Yes | No | Absent |
| TMEM220 | Q6QAJ8 | | | Yes | No | Absent |
| UNC93A | Q86WB7 | | | Yes | No | Absent |
| C4B | P0C0L5 | Medium | High | Yes | No | Absent |
| GBP7 | Q8N8V2 | | | Yes | No | Very low |
| C8A | P07357 | | | Yes | Yes | Very low |
| TMPRSS6 | Q8IU80 | | | Yes | No | Medium |
| CLEC1B | Q9P126 | | | Yes | No | Low |
| EFNA1 | P20827 | | | Yes | No | Low |
| PALM2 | Q8IXS6 | | | Yes | No | Low |
| PAQR9 | Q6ZVX9 | | | Yes | No | Low |
| SLC16A2 | P36021 | | | Yes | No | Low |
| SLC22A10 | Q63ZE4 | | | Yes | No | High |
| ABCG5 | Q9H222 | | | Yes | No | Absent |
| SLC17A1 | Q14916 | | | Yes | No | Absent |
| SMLR1 | H3BR10 | | | Yes | No | Absent |
| TRPC5 | Q9UL62 | | | Yes | No | Absent |
| TRPM8 | Q7Z2W7 | | | Yes | No | Absent |
| CLDN14 | O95500 | | | Yes | No | Absent |
| RTN4RL2 | Q86UN3 | | | Yes | No | Absent |
| GCGR | P47871 | | | Yes | No | Absent |
| EFNA2 | O43921 | | | Yes | No | Absent |
| IGSF23 | A1L1A6 | | | Yes | No | Absent |
| USH2A | O75445 | | | Yes | No | Absent |
| CHRNA4 | P43681 | | | Yes | No | Absent |
| MAMDC4 | Q6UXC1 | | | Yes | No | Absent |
| SIGLEC12 | Q96PQ1 | | | Yes | No | Absent |
| ART4 | Q93070 | | | Yes | No | Absent |
| AVPR1A | P37288 | | | Yes | No | Absent |
| CD302 | Q8IX05 | | | Yes | No | Absent |
| CNGA1 | P29973 | | | Yes | No | Absent |
| FYB2 | Q5VWT5 | | | Yes | No | Absent |
| GFRAL | Q6UXV0 | | | Yes | No | Absent |
| GRM8 | O00222 | | | Yes | No | Absent |
| PCSK9 | Q8NBP7 | | | Yes | No | Absent |
| HFE2 | Q6ZVN8 | | | Yes | No | Absent |
| IGSF9 | Q9P2J2 | | | Yes | No | Absent |
| NPBWR1 | P48145 | | | Yes | No | Absent |
| OR2T12 | Q8NG77 | | | Yes | No | Absent |
| OR2V1 | Q8NHB1 | | | Yes | No | Absent |
| OR4K17 | Q8NGC6 | | | Yes | No | Absent |
| RHOD | O00212 | | | Yes | No | Absent |
| RND1 | Q92730 | | | Yes | No | Absent |
| SLC10A5 | Q5PT55 | | | Yes | No | Absent |
| SLC17A9 | Q9BYT1 | | | Yes | No | Absent |
| SLC38A8 | A6NNN8 | | | Yes | No | Absent |
| SLC39A5 | Q6ZMH5 | | | Yes | No | Absent |
| SLCO1A2 | P46721 | | | Yes | No | Absent |
| TAS2R60 | P59551 | | | Yes | No | Absent |
| TENM1 | Q9UKZ4 | | | Yes | No | Absent |
| TMEM105 | Q8N8V8 | | | Yes | No | Absent |
| TMEM86B | Q8N661 | | | Yes | No | Absent |
| TRPV1 | Q8NER1 | | | Yes | No | Absent |
| OR10J5 | Q8NHC4 | | | Yes | Yes | Absent |
| OR2B2 | Q9GZK3 | | | Yes | Yes | Absent |
| SLC22A25 | Q6T423 | | | Yes | Yes | Absent |
| SERPINA1 | P01009 | | | Yes | No | Absent |

| | PROTEOMICS | PROTEOMICS | |
|---|---|---|---|
| Gene | DB | DB | Subcellular |
| Name | (LIVER) | (HEPG-2) | location |
| PLPPR2 | Absent | Absent | Membrane |
| SLC2A2 | High | Medium | Cell membrane |
| ABCB11 | Medium | Medium Low | Apical cell membrane |
| ABCB4 | Medium | Absent | Cell membrane |
| ASGR1 | High | High | Cell membrane |
| SLCO1B3 | Medium | Absent | Basolateral cell membrane; Multi- |
| | | | pass membrane protein. |
| TFR2 | Medium High | Absent | Cell membrane; Single-pass type II |
| | | | membrane protein.; |
| SLC22A7 | Medium | Absent | Basolateral cell membrane |
| GHR | Medium | Absent | Cell membrane; Single-pass type I |
| | | | membrane protein. Note = On growth |
| | | | hormone binding, GHR is |
| | | | ubiquitinated, internalized, down- |
| | | | regulated and transported into a |
| | | | degradative or non-degradative |
| | | | pathway |
| RNF130 | Medium | Absent | Membrane |
| SLC17A4 | NA | NA | Apical cell membrane |
| SLC13A5 | Medium High | Medium High | Membrane; Multi-pass membrane |
| | | | protein. Cell membrane |
| AQP9 | High | Absent | Cell membrane |
| ASGR2 | Medium High | High | Membrane; Single-pass type II |
| | | | membrane protein. |
| SLC10A1 | | | Membrane; Multi-pass |
| | | | membrane protein. |
| SLCO1B1 | Medium High | Absent | Basolateral cell membrane |
| ADGRG7 | | | Membrane |
| SLC38A3 | | | Cell membrane |
| FXYD1 | High | Medium low | Cell membrane, sarcolemma |
| TMPRSS9 | Medium | Absent | Cell membrane |
| SLC22A9 | Medium High | Absent | Basolateral cell membrane |
| PTP4A1 | Medium | High | Cell membrane |
| SLC38A4 | Medium | Absent | Cell membrane |
| CLEC4G | Medium High | Absent | Cell membrane; Single-pass |
| | | | type II membrane protein |
| TMEM82 | Medium | Absent | Membrane |
| ELFN1 | Medium low | Absent | Membrane |
| SLC7A2 | Medium | Medium | Cell membrane |
| TMEM56 | Medium | Absent | Membrane |
| RTP3 | Medium | Absent | Membrane |
| SLC17A2 | | | Membrane; Multi-pass |
| | | | membrane protein. |
| DNAJC22 | Medium low | Absent | Membrane |
| LRRC3 | Low | Absent | Membrane |
| TMEM220 | Medium | Absent | Membrane |
| UNC93A | Medium | Absent | Cell membrane |
| C4B | | | Secreted. Cell junction, synapse |
| GBP7 | Medium | Absent | Membrane |
| C8A | Medium | Low | Secreted. Cell membrane; Multi- |
| | | | pass membrane protein. |
| | | | Note = Secreted as soluble protein. |
| | | | Inserts into the cell membrane of |
| | | | target cells. |
| TMPRSS6 | Medium low | Absent | Cell membrane |
| CLEC1B | Medium | Absent | Membrane |
| EFNA1 | Medium | Absent | Cell membrane |
| PALM2 | Medium | Absent | Cell membrane |
| PAQR9 | Medium | Absent | Cell membrane |
| SLC16A2 | Medium | Absent | Cell membrane |
| SLC22A10 | | | Membrane |
| ABCG5 | | | Cell membrane |
| SLC17A1 | | | Apical cell membrane |
| SMLR1 | | | Membrane |
| TRPC5 | | | Cell membrane |
| TRPM8 | | | Cell membrane; Multi-pass |
| | | | membrane protein. Membrane raft. |
| | | | Endoplasmic reticulum membrane. |
| | | | Note = Localizes to membrane rafts |
| | | | but is also located in the cell |
| | | | membrane outside of these regions |
| | | | where channel response to cold is |
| | | | enhanced compared to membrane |
| | | | rafts (By similarity). Located |
| | | | in the endoplasmic reticulum |
| | | | in prostate cancer cells. |
| CLDN14 | | | Cell junction, tight junction. |
| | | | Cell membrane; Multi-pass |
| | | | membrane protein. |
| RTN4RL2 | | | Cell membrane |
| GCGR | | | Cell membrane |
| EFNA2 | | | Cell membrane |
| IGSF23 | | | Membrane |
| USH2A | | | Cell projection, stereocilium |
| | | | membrane |
| CHRNA4 | | | Cell junction, synapse, |
| | | | postsynaptic cell membrane; Multi- |
| | | | pass membrane protein. Cell |
| | | | membrane; Multi-pass membrane |
| | | | protein. Cell membrane |
| MAMDC4 | Low | Absent | Membrane |
| SIGLEC12 | | | Membrane; Single-pass type I |
| | | | membrane protein. |
| ART4 | Medium | Absent | Cell membrane; Lipid-anchor, |
| | | | GPI-anchor. |
| AVPR1A | | | Cell membrane; Multi-pass |
| | | | membrane protein. |
| CD302 | | | Membrane |
| CNGA1 | | | Cell membrane |
| FYB2 | | | Membrane raft |
| GFRAL | | | Cell membrane |
| GRM8 | | | Cell membrane; Multi-pass |
| | | | membrane protein. |
| PCSK9 | Low | Absent | Cytoplasm. Secreted. Endosome. |
| | | | Lysosome. Cell surface. |
| | | | Endoplasmic reticulum. Golgi |
| | | | apparatus. Note = Autocatalytic |
| | | | cleavage is required to transport it |
| | | | from the endoplasmic reticulum to |
| | | | the Golgi apparatus and for the |
| | | | secretion of the mature protein. |
| | | | Localizes to the endoplasmic |
| | | | reticulum in the absence of LDLR |
| | | | and colocalizes to the cell surface |
| | | | and to the endosomes/lysosomes in |
| | | | the presence of LDLR. The sorting |
| | | | to the cell surface and endosomes |
| | | | is required in order to fully promote |
| | | | LDLR degradation. |
| HFE2 | | | Cell membrane |
| IGSF9 | Medium | Absent | Cell membrane |
| NPBWR1 | | | Cell membrane; Multi-pass |
| | | | membrane protein. |
| OR2T12 | | | Cell membrane; Multi-pass |
| | | | membrane protein. |
| OR2V1 | | | Cell membrane; Multi-pass |
| | | | membrane protein. |
| OR4K17 | | | Cell membrane; Multi-pass |
| | | | membrane protein. |
| RHOD | Medium | Absent | Cell membrane |
| RND1 | | | Cell membrane |
| SLC10A5 | | | Membrane |
| SLC17A9 | Absent | Medium | Membrane |
| SLC38A8 | | | Membrane |
| SLC39A5 | Medium | Absent | Basolateral cell membrane |
| SLCO1A2 | | | Cell membrane; Multi-pass |
| | | | membrane protein. |
| TAS2R60 | | | Membrane; Multi-pass |
| | | | membrane protein. |
| TENM1 | | | Cell membrane |
| TMEM105 | | | Membrane |
| TMEM86B | Medium | Absent | Membrane |
| TRPV1 | | | Cell junction, synapse, |
| | | | postsynaptic cell membrane |
| OR10J5 | | | Cell membrane; Multi-pass |
| | | | membrane protein. |
| OR2B2 | | | Cell membrane; Multi-pass |
| | | | membrane protein. |
| SLC22A25 | | | Membrane |
| SERPINA1 | | | Secreted. Endoplasmic reticulum. |
| | | | Note = The S and Z allele are not |
| | | | secreted effectively and accumulate |
| | | | intracellularly in the endoplasmic |
| | | | reticulum.; |

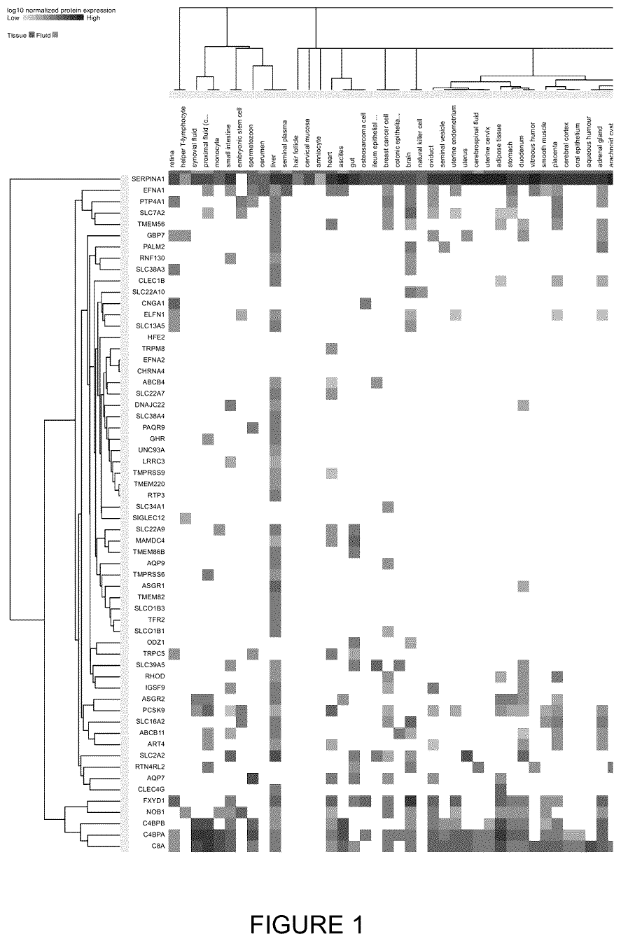

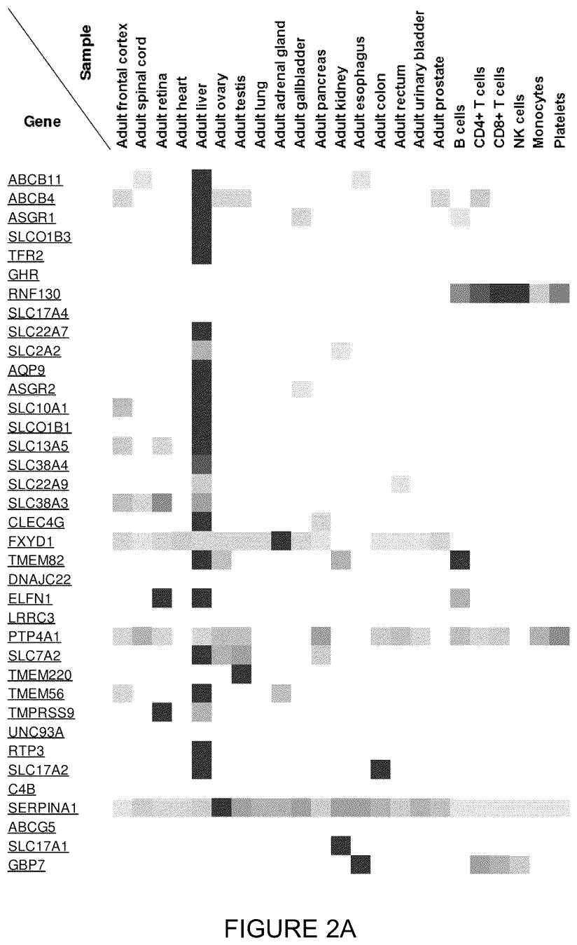

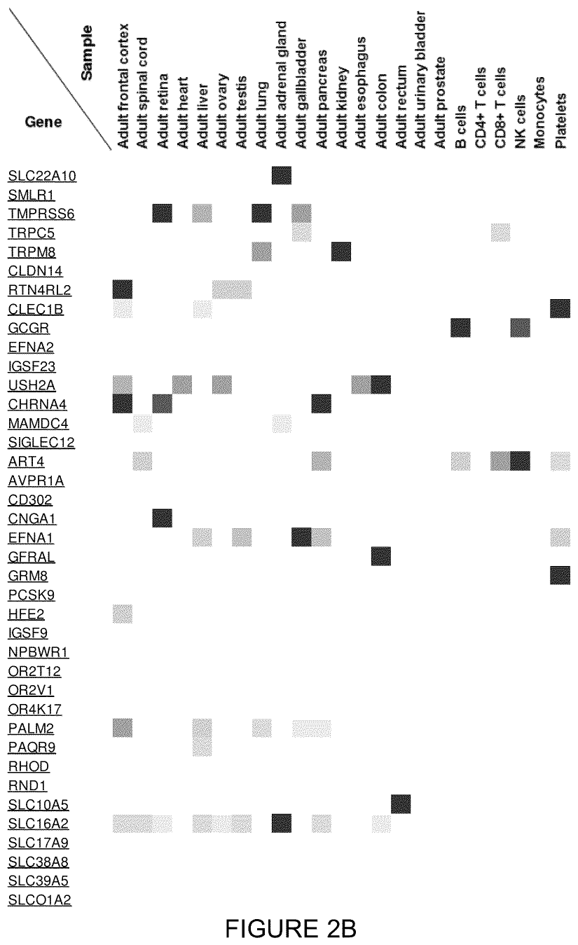

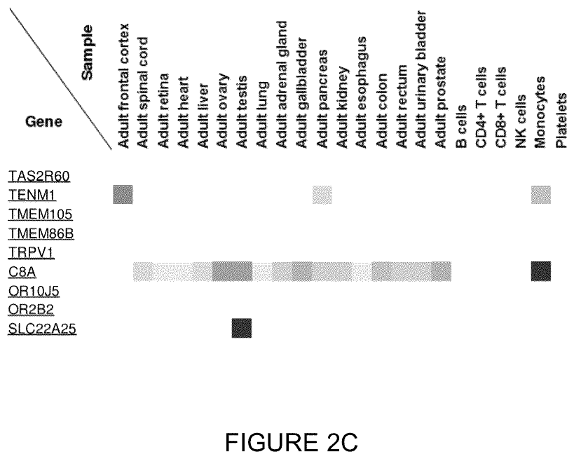

Heatmaps with the protein expression profile were extracted from both ProteomicsDB and Human Proteome Map of the initial list of 88 membrane proteins after UniProt subcellular location annotation (FIG. 1 and FIG. 2).

ProteomicsDB and Human Proteome Map expression profile information were used to rank the proteins in terms of tissue specificity further, alongside the information previously obtained from The Human Protein Atlas (latest accessed on 11/2021). A total of 34 candidate biomarkers were taken forward (Table 2).

TABLE 2
Top 34 candidate biomarkers

| | | | | Human | (Human | |
|---|---|---|---|---|---|---|
| | | Human | Human | Protein | Protein | |
| | | Protein | Protein | Atlas | Atlas) | |
| | | Atlas | Atlas | (mRNA | mRNA only | |
| Gene | Uniprot | (Protein | (Protein | overexpressed | expressed | Human |
| Name | ID | expression) | specificity) | in liver) | in liver | proteome |
| ABCB11 | O95342 | High | High | Yes | No | High |
| ABCB4 | P21439 | High | High | Yes | No | High |
| ASGR1 | P07306 | High | High | Yes | No | High |
| SLCO1B3 | Q9NPD5 | High | High | Yes | No | High |
| TFR2 | Q9UP52 | High | High | Yes | No | High |
| GHR | P10912 | High | Medium | No | No | Absent |
| PLPPR2 | Q96GM1 | High | Medium | No | No | NA |
| RNF130 | Q86XS8 | High | Medium | No | No | Absent |
| SLC17A4 | Q9Y2C5 | High | Medium | No | No | Absent |
| SLC22A7 | Q9Y694 | High | Medium | No | No | High |
| SLC2A2 | P11168 | High | Medium | No | No | Medium |
| AQP9 | O43315 | Medium | High | Yes | No | High |
| ASGR2 | P07307 | Medium | High | Yes | No | High |
| SLC10A1 | Q14973 | Medium | High | Yes | No | High |
| SLCO1B1 | Q9Y6L6 | Medium | High | Yes | Yes | High |
| SLC13A5 | Q86YT5 | Low | High | Yes | No | High |
| ADGRG7 | Q96K78 | | | Yes | No | NA |
| SLC38A4 | Q969I6 | | | Yes | No | High |
| SLC22A9 | Q8IVM8 | | | Yes | No | Low |
| SLC38A3 | Q99624 | | | Yes | No | Medium |
| CLEC4G | Q6UXB4 | | | Yes | No | High |
| FXYD1 | O00168 | | | Yes | No | Medium |
| TMEM82 | A0PJX8 | | | Yes | No | High |
| DNAJC22 | Q8N4W6 | | | Yes | No | Absent |
| ELFN1 | P0C7U0 | | | Yes | No | High |
| LRRC3 | Q9BY71 | | | Yes | No | Absent |
| PTP4A1 | Q93096 | | | Yes | No | Low |
| SLC7A2 | P52569 | | | Yes | No | High |
| TMEM220 | Q6QAJ8 | | | Yes | No | Absent |
| TMEM56 | Q96MV1 | | | Yes | No | High |
| TMPRSS9 | Q7Z410 | | | Yes | No | Medium |
| UNC93A | Q86WVB7 | | | Yes | No | Absent |
| RTP3 | Q9BQQ7 | | | Yes | Yes | High |
| SLC17A2 | O00624 | | | Yes | Yes | High |

| | PROTEOMICS | PROTEOMICS | |
|---|---|---|---|
| Gene | DB | DB | Subcellular |
| Name | (LIVER) | (HEPG-2) | location |
| ABCB11 | Medium | Medium Low | Apical cell membrane |
| ABCB4 | Medium | Absent | Cell membrane |
| ASGR1 | High | High | Cell membrane |
| SLCO1B3 | Medium | Absent | Basolateral cell membrane; Multi- |
| | | | pass membrane protein. |
| TFR2 | Medium High | Absent | Cell membrane; Single-pass type II |
| | | | membrane protein.; |
| GHR | Medium | Absent | Cell membrane; Single-pass type I |
| | | | membrane protein. Note = On growth |
| | | | hormone binding, GHR is |
| | | | ubiquitinated, internalized, down- |
| | | | regulated and transported into a |
| | | | degradative or non-degradative |
| | | | pathway. |
| PLPPR2 | Absent | Absent | Membrane |
| RNF130 | Medium | Absent | Membrane |
| SLC17A4 | NA | NA | Apical cell membrane |
| SLC22A7 | Medium | Absent | Basolateral cell membrane |
| SLC2A2 | High | Medium | Cell membrane |
| AQP9 | High | Absent | Cell membrane |
| ASGR2 | Medium High | High | Membrane; Single-pass type II |
| | | | membrane protein. |
| SLC10A1 | | | Membrane; Multi-pass membrane |
| | | | protein. |
| SLCO1B1 | Medium High | Absent | Basolateral cell membrane |
| SLC13A5 | Medium High | Medium High | Membrane; Multi-pass membrane |
| | | | protein. Cell membrane |
| ADGRG7 | | | Membrane |
| SLC38A4 | Medium | Absent | Cell membrane |
| SLC22A9 | Medium High | Absent | Basolateral cell membrane |
| SLC38A3 | | | Cell membrane |
| CLEC4G | Medium High | Absent | Cell membrane; Single-pass type II |
| | | | membrane protein |
| FXYD1 | High | Medium low | Cell membrane, sarcolemma |
| TMEM82 | Medium | Absent | Membrane |
| DNAJC22 | Medium low | Absent | Membrane |
| ELFN1 | Medium low | Absent | Membrane |
| LRRC3 | Low | Absent | Membrane |
| PTP4A1 | Medium | High | Cell membrane |
| SLC7A2 | Medium | Medium | Cell membrane |
| TMEM220 | Medium | Absent | Membrane |
| TMEM56 | Medium | Absent | Membrane |
| TMPRSS9 | Medium | Absent | Cell membrane |
| UNC93A | Medium | Absent | Cell membrane |
| RTP3 | Medium | Absent | Membrane |
| SLC17A2 | | | Membrane; Multi-pass membrane |
| | | | protein. |

### 1-2) Validation of Potential Candidate Biomarkers of Liver Tissue

### Materials and Methods:

Collection of EVs from Cultured Cells

The HCC cell line HepG2 was grown in growth medium Advanced-DMEM, supplemented with 5% (v/v) FBS and 4 mM glutaMAX, in a humidifying atmosphere with 5% CO2 and 37° C. After reaching 70% confluency in T175 flaks, the cells were gently washed three times with PBS and were cultured in Advanced-DMEM FBS-free medium.

After 48 hrs, the conditioned medium was collected and centrifuged for 5 min at 300 g, followed by another centrifugation of 10 min at 3000 g and 4° C. to remove cellular debris and other large contaminants. The conditioned medium was then filtered using 0.22 μm PES filters and concentrated down to 500 μL with Amicon Ultra-15 100K MWCO Centrifugal filters. The EVs were finally isolated by size exclusion chromatography using IZON qEV original/70 nm columns, on an Automatic Fraction Collector (AFC), according to the supplier's instructions.

The first three fractions which were eluted, corresponding to a total of 1.5 mL of elution volume, were collected, pooled together, aliquoted and stored at −80° C. until further use.

Collection of EVs from Tissue Samples

Frozen liver tissue were acquired from specialised biobanks. To prepare the tissue for EV isolation, 2 mL of Advanced-DMEM was added to 0.2 mg of tissue cut in five smaller pieces. Collagenase D and DNase I was added to a final concentration of 2 mg/ml and 40 U/ml, respectively, and incubated for 30 min at 37° C. with gentle rotation. To remove large debris, the solution was filtered through a 70 μM strainer and 20 mL of sterile PBS were used to wash the strainer. Furthermore, a series of centrifugations of 500, 3000 and 10000 g were performed during 10, 20 and 30 min, respectively, and the pellet formed was discarded in every step. Finally, the sample was filtered through a 0.22 μM to remove large vesicles and other contaminants and concentrated down to 1 mL with Amicon Ultra-15 100K MWCO Centrifugal filters.

The EVs were isolated by size exclusion chromatography using Izon qEV GEN2 35 nm columns on an Automatic Fraction Collector (AFC), according to the supplier's instructions.

The first two fractions which were eluted, corresponding to a total of 2.4 mL of elution volume, were collected, pooled together, aliquoted and stored at −80° C. until further use. Isolated EVs were characterised by nanoparticle tracking analysis

Particle number and size distribution of extracellular vesicle preparations were determined by Nanoparticle Tracking Analysis (NTA) using a NanoSight NS300 system (Malvern) configured with a 488 nm laser and a high sensitivity scientific CMOS camera.

Samples were diluted in particle-free PBS, to an acceptable concentration, according to the manufacturer's recommendations. The samples were analysed under constant flow rate and 3×60 sec videos were captured with a camera level of 12. Data was analysed using NTA 3.4 software with a detection threshold of 7.

### Preparation of Antibody-Labelled-Magnetic Beads

Magnetic beads (Dynabeads® M-270 Epoxy beads) were coupled to the following antibodies using the Dynabeads™ Antibody Coupling Kit: anti-CD63 antibodies (orb506484, Biorbyt; anti-ABCB11 (sc-74500, Santa Cruz Biotechnology), anti-ABCB11 antibodies (STJ190611, St John's Laboratory); anti-ABCB4 antibodies (No. ABIN6388963, antibodies-online.com); anti-ABCB4 antibodies (CSB-PA001050LA01HU, Cusabio); anti-ASGR1 antibodies (NBP1-60150, Novus Biologicals); anti-ASGR1 antibodies (MAB43941-100, R&D Systems); anti-ASGR1 antibodies (ab254262, Abcam); anti-ASGR1 antibodies (sc-52623, Santa Cruz Biotechnology); anti-TFR2 antibodies (MAB3120, Novus Biologicals); anti-TFR2 antibodies (ab13579, Abcam); anti-GHR antibodies (STJ97268, St John's Laboratory); anti-RNF130 antibodies (orb1291, Biorbyt); anti-SLC17A4 antibodies (sc-135562, Santa Cruz Biotechnology); anti-SLC2A2 antibodies (orb33144, Biorbyt); anti-SLC2A2 (CSB-PA13329A0Rb, Cusabio); anti-SLC2A2 antibodies (sc-518022, Santa Cruz Biotechnology); anti-AQP9 antibodies (ABIN2746270, antibodies-online.com); anti-ASGR2 antibodies (orb757191, Biorbyt); anti-ASGR2 antibodies (ab200196, Abcam); anti-SLC10A1 antibodies (sc-518115, Santa Cruz Biotechnology); anti-SLCO1B1 antibodies (CSB-PA896932LA01HU, Cusabio); anti-SLCO1B1 antibodies (sc-271157, Santa Cruz Biotechnology); anti-SLCO1B1 antibodies (STJ94587, St John's Laboratory); anti-SLC13A5 antibodies (CSB-PA768239LA01HU, Cusabio); anti-SLC13A5 antibodies (sc-293277, Santa Cruz Biotechnology); anti-SLC38A4 antibodies (sc-515125, Santa Cruz Biotechnology); anti-SLC38A3 antibodies (sc-398982, Santa Cruz Biotechnology); anti-SLC38A3 antibodies (orb37057, Biorbyt); anti-SLC22A9 antibodies (CSB-PA811607LA01HU, Cusabio); anti-CLEC4G antibodies (MAB2947, R&D Systems); anti-CLEC4G antibodies (sc-65478, Santa Cruz Biotechnology); anti-CLEC4G antibodies (CSB-PA744269LA01HU, Cusabio); anti-FXYD1 antibodies (sc-393415, Santa Cruz Biotechnology); anti-ELFN1 antibodies (MAB10644, R&D Systems); anti-LRRC3 antibodies (MAB5039, R&D Systems); anti-PTP4A1 antibodies (sc-130354, Santa Cruz Biotechnology); anti-TMEM56 antibodies (CSB-PA021714LA01HU, Cusabio); anti-UNC93A antibodies (sc-390157, Santa Cruz Biotechnology); anti-UNC93A antibodies (CSB-PA773046LA01HU, Cusabio); anti-RTP3 antibodies (CSB-PA887106LA01HU, Cusabio); anti-SLC17A2 antibodies (CSB-PA004099, Cusabio); anti-HEPCAM antibodies (anti-sc-515637, Santa Cruz Biotechnology); anti-ABCC6 antibodies (ab167564, Abcam); or anti-APOE antibodies (sc-13521, Santa Cruz Biotechnology).

Briefly, the magnetic beads were washed as per the kit instructions and incubated overnight at 37° C., under constant rotation in LoBind microcentrifuge tubes (Eppendorf). The amount of capture antibody per each of the reactions was 10 μg per mg of magnetic beads. Tween®20 (0.05%) was added in HB and LB wash buffers for improved stringency as recommended by the supplier. Washings of the magnetic beads were performed with a DynaMag-Spin Magnet (Invitrogen).

### Detection of Antibody-Labelled-Magnetic Bead-Captured EVs

After isolation of EVs by differential centrifugation, ultrafiltration and size exclusion chromatography, the cell culture-derived EVs were normalized to a concentration of 7×10⁸ part/mL in PBS and the tissue-derived EVs were normalized to a concentration of 1×10¹⁰ part/mL in 20% Assay Defender (Candor Bioscience).

Cell-derived EVs and tissue-derived EVs were captured with magnetic beads pre-coated with the following antibodies in individual reactions: anti-CD63 antibodies; anti-ABCB1 antibodies; anti-ABCB11 antibodies; anti-ABCB4 antibodies; anti-ABCB4 antibodies; anti-ASGR1 antibodies; anti-ASGR1 antibodies; anti-ASGR1 antibodies; anti-ASGR1 antibodies; anti-TFR2 antibodies; anti-TFR2 antibodies; anti-GHR antibodies; anti-RNF130 antibodies; anti-SLC17A4 antibodies; anti-SLC2A2 antibodies; anti-SLC2A2 antibodies; anti-SLC2A2 antibodies; anti-AQP9 antibodies; anti-ASGR2 antibodies; anti-ASGR2 antibodies; anti-SLC10A1 antibodies; anti-SLCO1B antibodies; anti-SLCO1B1 antibodies; anti-SLCO1B1 antibodies; anti-SLC13A5 antibodies; anti-SLC13A5 antibodies; anti-SLC38A4 antibodies; anti-SLC38A3 antibodies; anti-SLC38A3 antibodies; anti-SLC22A9 antibodies; anti-CLEC4G antibodies; anti-CLEC4G antibodies; anti-CLEC4G antibodies; anti-FXYD1 antibodies; anti-ELFN1 antibodies; anti-LRRC3 antibodies; anti-PTP4A1 antibodies; anti-TMEM56 antibodies; anti-UNC93A antibodies; anti-UNC93A antibodies; anti-RTP3 antibodies; anti-SLC17A2 antibodies; anti-HEPCAM antibodies; anti-ABCC6 antibodies; or anti-APOE antibodies.

Magnetic beads pre-coated with anti-CD63 antibodies, as prepared according to Example 1-2), were used as a control. Samples were mixed every 30 min and, after 12 hr incubation, magnetic bead-captured EVs were recovered by the washing of the samples in a magnetic field using the KingFisher.

For all experiments, biotinylated anti-CD63 antibodies were used for signal detection. Biotinylated anti-CD63 antibodies were incubated at 10 ng/μL with the magnetic bead-captured EVs for 60 min. Then, the magnetic beads were washed and 200 μL of Poly-HRP strep diluted as 1:6000 was incubated with the samples for 30 min. After washing, 100 μL QuantaBlu™ was added to each of the samples and incubated for 40 minutes. The reactions were stopped by the addition of 100 μL of stop solution and 190 μL of each sample was transferred to a F-shaped 96-well plate for signal measurement at Ex/Em=320/405 nm.

### Results:

EVs were collected from the HEPG-2 HCC cell culture and HCC tissue samples and the EVs were subsequently captured and isolated by targeting the top 34 candidate biomarkers identified in the previous step (Tables 3 and 4 respectively).

TABLE 3
HEPG-2 2 × 10⁹ part/mL

| Signal with background subtracted (S-N) | | Signal-to-noise ratio (S/N) | |
|---|---|---|---|
| Marker (ab) | Value | Marker (ab) | Value |
| ASGR1 (MAB43941-100) | 2.85 | ASGR2 (ab200196) | 17.4 |
| SLC38A3 (sc-398982) | 2.65 | ASGR1 (MAB43941-100) | 14.99 |
| FXYD1 (sc-393415) | 1.59 | SLCO1B1 (sc-271157) | 8.49 |
| ASGR2 (ab200196) | 1.3 | SLC38A3 (sc-398982) | 8.27 |
| CLEG4 (CSB- | 1.24 | FXYD1 (sc-393415) | 4.65 |
| PA744269LA01HU) | | | |
| SLCO1B1 (sc-271157) | 1.03 | CLEG4 (CSB-PA744269LA01HU) | 3.99 |
| SLCO1B3 (ABIN570800) | 0.6 | TFR2 (MAB3120) | 3.15 |
| LRRC3 (MAB5039) | 0.54 | SLC2A2 (sc-518022) | 3.03 |
| ELFN1 (MAB10644) | 0.35 | SLC17A4 (sc-135562) | 3.01 |
| SLC17A4 (sc-135562) | 0.34 | ELFN1 (MAB10644) | 2.52 |
| SLC22A9 (CSB- | 0.32 | SLC22A9 (CSB- | 2.18 |
| PA811607LA01HU) | | PA811607LA01HU) | |
| ABCB11 (sc-74500) | 0.29 | ABCC6 (ab167564) | 1.96 |
| SLCO1B1 (CSB- | 0.22 | | |
| PA896932LA01HU) | | | |
| SLC2A2 (sc-518022) | 0.19 | | |
| SLC13A5 (CSB- | 0.19 | | |
| PA768239LA01HU) | | | |
| RTP3 (CSB-PA887106LA01HU) | 0.18 | | |
| TFR2 (MAB3120) | 0.16 | | |
| ABCC6 (ab167564) | 0.11 | | |

TABLE 4
Tissue HCC1 1 × 10¹⁰ part/mL

| Signal with background subtracted (S/N) | | Signal-to-noise ratio (S/N) | |
|---|---|---|---|
| Marker (ab) | Value | Marker (ab) | Value |
| ASGR2 (ab200196) | 2.93 | ASGR2 (ab200196) | 24.37 |
| ASGR1 (MAB43941-100) | 2.34 | CLEG4 (CSB-PA744269LA01HU) | 7.05 |
| TFR2 (MAB3120) | 1.58 | TFR2 (MAB3120) | 6.98 |
| FXYD1 (sc-393415) | 1.57 | UNC93A (CSB-PA773046LA01HU) | 6.19 |
| SLC38A3 (sc-398982) | 1.55 | FXYD1 (sc-393415) | 5.26 |
| CLEG4 (sc-65478) | 1.2 | GHR (STJ97268) | 4.87 |
| GHR (STJ97268) | 1.16 | TMEM56 (CSB-PA023857LA01HU) | 4.85 |
| CLEG4 (CSB- | 1.13 | SLCO1B1 (sc-271157) | 4.74 |
| PA744269LA01HU) | | | |
| APOE (sc-13521) | 1.12 | SLC2A2 (CSB-PA13329A0Rb) | 3.91 |
| SLC2A2 (CSB-PA13329A0Rb) | 1.1 | ASGR1 (MAB43941-100) | 3.45 |
| UNC93A (CSB- | 1.01 | CLEG4 (CSB-PA744269LA01HU) | 3.06 |
| PA773046LA01HU) | | | |
| TMEM56 (CSB- | 0.98 | SLC2A2 (sc-518022) | 3.05 |
| PA023857LA01HU) | | | |
| SLCO1B1 (sc-271157) | 0.97 | SLCO1B1 (CSB- | 2.88 |
| | | PA896932LA01HU) | |
| SLCO1B1 (CSB- | 0.91 | SLC22A9 (CSB- | 2.87 |
| PA896932LA01HU) | | PA811607LA01HU) | |
| SLC2A2 (sc-518022) | 0.82 | SLC38A3 (sc-398982) | 2.83 |
| SLC10A1 (sc-518115) | 0.81 | ELFN1 (MAB10644) | 2.77 |
| SLC22A9 (CSB- | 0.8 | RFN130 (orb1291) | 2.27 |
| PA811607LA01HU) | | | |
| SLCO1B3 (ABIN570800) | 0.79 | ABCC6 (ab167564) | 2.24 |
| SLC17A2 (CSB-PA004099) | 0.68 | SLC38A4 (sc-515125) | 2.13 |
| HEPACAM (sc-515637) | 0.49 | SLC10A1 (sc-518115) | 2.11 |
| UNC93A (sc-390157) | 0.48 | HEPACAM (sc-515637) | 2.01 |
| SLC38A4 (sc-515125) | 0.47 | APOE (sc-13521) | 1.82 |
| RTP3 (CSB-PA887106LA01HU) | 0.38 | SLCO1B3 (ABIN570800) | 1.71 |
| ELFN1 (MAB10644) | 0.38 | PTP4A1 (sc-130354) | 1.69 |
| RFN130 (orb1291) | 0.32 | UNC93A (sc-390157) | 1.65 |
| PTP4A1 (sc-130354) | 0.29 | | |
| Isotype | 0.2 | | |

Taking into account the functional importance of the biomarker in the liver, the candidate biomarkers showing a signal to noise ratio higher than 2.5 with a signal to noise difference higher than 0.5 O.D. in either liver tissue or liver cell lines derived EVs were taken forward (Table 5).

TABLE 5
Top 11 candidate biomarkers

| | | | | Human | (Human | | | |
|---|---|---|---|---|---|---|---|---|
| | | Human | Human | Protein | Protein | | | |
| | | Protein | Protein | Atlas | Atlas) | | | |
| | | Atlas | Atlas | (mRNA | mRNA only | | PROTEOMICS | PROTEOMICS |
| Gene | Uniprot | (Protein | (Protein | overexpressed | expressed | Human | DB | DB |
| Name | ID | expression) | specificity) | in liver) | in liver | proteome | (LIVER) | (HEPG-2) |
| ASGR1 | P07306 | High | High | Yes | No | High | High | High |
| TFR2 | Q9UP52 | High | High | Yes | No | High | Medium High | Absent |
| SLC2A2 | P11168 | High | Medium | No | No | Medium | High | Medium |
| ASGR2 | P07307 | Medium | High | Yes | No | High | Medium High | High |
| SLCO1B1 | Q9Y6L6 | Medium | High | Yes | Yes | High | Medium High | Absent |
| SLC22A9 | Q8IVM8 | | | Yes | No | Low | Medium High | Absent |
| SLC38A3 | Q99624 | | | Yes | No | Medium | | |
| CLEC4G | Q6UXB4 | | | Yes | No | High | Medium High | Absent |
| FXYD1 | O00168 | | | Yes | No | Medium | High | Medium low |
| TMEM56 | Q96MV1 | | | Yes | No | High | Medium | Absent |
| UNC93A | Q86WB7 | | | Yes | No | Absent | Medium | Absent |

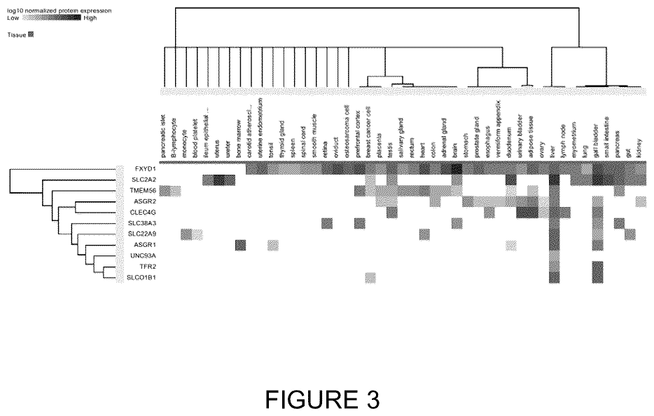

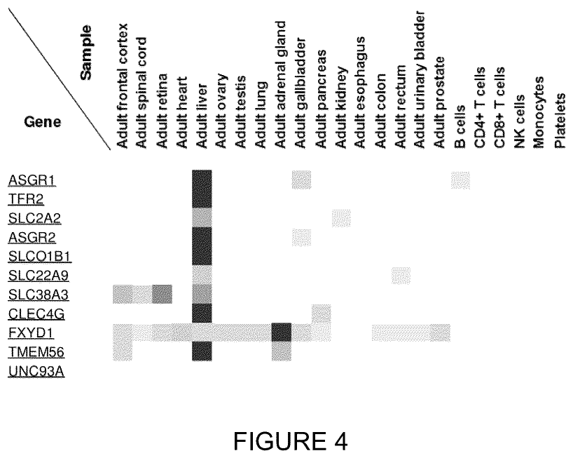

Heatmaps with the protein expression profile were extracted from both ProteomicsDB and Human Proteome Map of the 11 proteins for which the respective antibodies demonstrated high signal following the capture of EVs collected from HCC cell line and HCC tumour tissue (FIG. 3 and FIG. 4).

### 1-3) Detection of Candidate Biomarkers

### Materials and Methods:

Collection of EVs from Cultured Cells

The HCC cell line HepG2 (ATCC, catalogue no. HB-8065) and the breast cancer cell lines MCF-7 (ATCC, catalogue no. HTB-22) and BT-474 (ATCC, catalogue no. HTB-20) were cultured and EVs collected according to Example 1-2).

Collection of EVs from Tissue Samples

Frozen lung, kidney and liver tissue were acquired from specialised biobanks and EVs collected according to Example 1-2).

Collection of EVs from Human Plasma

Frozen human plasma derived from whole blood in K2EDTA Vacutainers was acquired (Cambridge bioscience). The human plasma was ethically consented and originated from a pool of healthy, paid volunteers (Research Donors). Cirrhosis and HCC patient whole blood in K2EDTA Vacutainers were prospectively collected by the BIOBANCO-Molecular Medicine Institute/Santa Maria Hospital and UCL Biobank-Royal Free London NHS Foundation Trust (RFL B-ERC) and ethically consented.

The human plasma was slowly thawed on ice and divided into two portions of 2 mL each. One of the portions was spiked with BT-474 EVs, reaching to a final concentration of 1.67E9 part/mL. Then, both the spiked and non-spiked samples were treated equally by a centrifugation step of 15 min, at 3000 g and at 4° C. followed by another centrifugation of 20 min at 10000 g and 4° C. to remove any cell debris, large particles, or aggregates.

Then, the samples were filtered using a 0.22 μm PES filter and 1.8 mL of each sample was processed by size exclusion chromatography using an Izon qEV2 70 nm column on an AFC. As for cell culture preparations, the first three fractions of each sample which were eluted, and corresponding to a total of 5.4 mL per sample, were collected and pooled together.

Isolated EVs were Characterised by Nanoparticle Tracking Analysis

Particle number and size distribution of MCF-7 and BT-474 and extracellular vesicle preparations were determined according to Example 1-2).

### Preparation of Antibody-Labelled-Magnetic Beads

Antibody-Labelled-Magnetic Beads were Prepared According to Example 1-2).

### Detection of Antibody-Labelled-Magnetic Bead-Captured EVs

After isolation of EVs by differential centrifugation, ultrafiltration and size exclusion chromatography, the various cell culture-derived EVs were normalized to a concentration of 7×10⁸ part/mL in PBS and the tissue-derived EVs were normalized to a concentration of 1×10¹⁰ part/mL in 20% Assay Defender. Isolated plasma EVs were diluted in 20% Assay Defender.

Cell-derived EVs were captured with magnetic beads pre-coated with the following antibodies, as prepared according to Example 1-2): anti-ASGR1 antibodies, anti-TFR2 antibodies, anti-SLC2A2 antibodies, anti-ASGR2 antibodies, anti-SLCO1B1 antibodies, anti-SLC22A9 antibodies, anti-SLC38A3 antibodies, anti-CLEC4G antibodies, anti-FXYD1 antibodies, anti-TMEM56 antibodies or anti-UNC93A antibodies.

Tissue-derived EVs were captured with magnetic beads pre-coated with the following antibodies, as prepared according to Example 1-2): anti-ASGR1 antibodies, anti-TFR2 antibodies, anti-SLC2A2 antibodies, anti-ASGR2 antibodies, anti-SLCO1B1 antibodies, anti-SLC22A9 antibodies, anti-SLC38A3 antibodies, anti-CLEC4G antibodies, anti-FXYD1 antibodies, anti-TMEM56 antibodies or anti-UNC93A antibodies.

Isolated plasma EVs were captured with magnetic beads pre-coated with anti-ASGR1 antibodies, anti-ASGR2 antibodies, anti-TRF1 antibodies or anti-SLCO1B1 antibodies, as prepared according to Example 1-2), in individual reactions for each of the antibody pre-coated magnetic beads preparation or mixed together at a ratio of 4:2:2:2 ASGR1: ASGR2: TRF1: SLCO1B1 and incubated in a KingFisher Flex System.

### Results:

### Detection of the Candidate Biomarkers in Cultured Cells

EVs were collected from HCC and breast cancer cell cultures and were subsequently captured and isolated by targeting the top 11 candidate biomarkers identified in the previous bioinformatics step (Table 5).

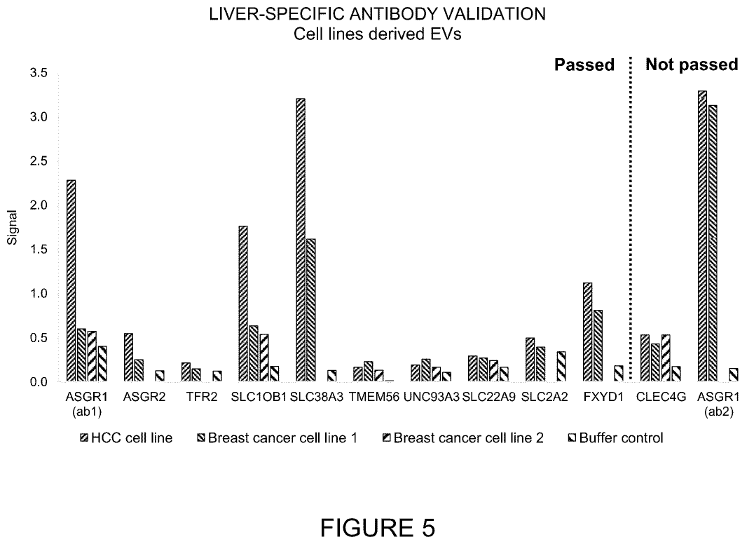

As shown in FIG. 5, the capture of EVs using magnetic beads pre-coated with anti-ASGR1 antibodies (ab1), anti-ASGR2 antibodies, anti-TFR2 antibodies, anti-SLCO1B1 antibodies, anti-SLC38A3 antibodies, anti-TMEM56 antibodies, anti-UNC93A antibodies, anti-SLC22A9 antibodies, anti-SLC2A2 antibodies or anti-FXYD1 antibodies resulted in a higher signal from HCC cell lines in comparison to breast cancer cell lines 1 (MCF-7) and 2 (BT-474) and the buffer control. In contrast, no significant differences were observed for EVs isolated from the different cell cultures using magnetic beads pre-coated with anti-CLEC4G antibodies or a second anti-ASGR1 antibody (ab2).

The candidate biomarkers showing a higher signal for EVs isolated from the HCC cell line in comparison to the non-target breast cancer cell lines 1 and 2 and the buffer control passed the validation and were taken forward for detection in tissue samples.

Those biomarkers which did not result in any significant differences in the signal of the CD63 surface marker on EVs between the HCC cell lines, breast cancer cell lines 1 and 2 and the buffer control did not pass the validation.

### Detection of the Candidate Biomarkers in Tissue Sample

EVs were collected from various snap-frozen human tissue samples. EVs were then captured and isolated by targeting the different candidate biomarkers identified from the cell culture experiments described above.

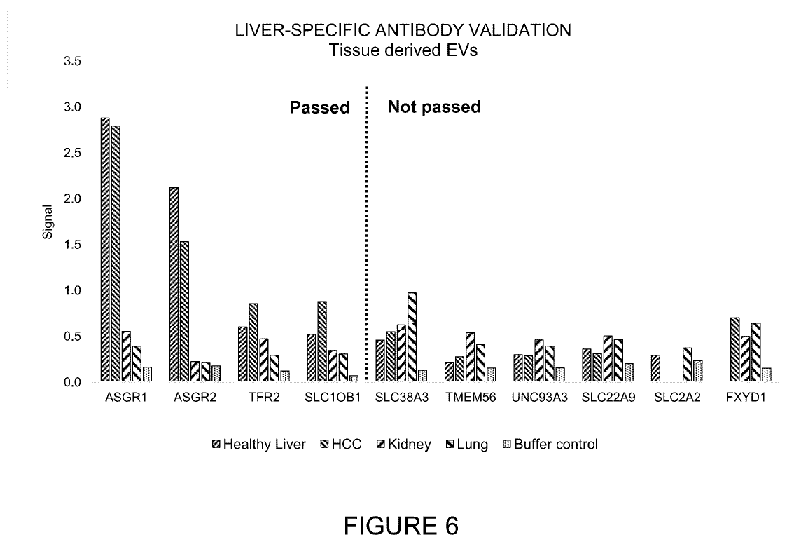

As shown in FIG. 6, the capture of EVs using magnetic beads pre-coated with anti-ASGR1 antibodies (ab1), anti-ASGR2 antibodies, anti-TFR2 antibodies or anti-SLCO1B1 antibodies resulted in a higher signal from healthy liver tissue and HCC tissue in comparison to kidney tissue, lung tissue and the buffer control. In contrast, no significant differences were observed for EVs isolated from the different tissue types using magnetic beads pre-coated with anti-SLC38A3 antibodies, anti-TMEM56 antibodies, anti-UNC93A antibodies, anti-SLC22A9 antibodies, anti-SLC2A2 antibodies or anti-FXYD1 antibodies.

The candidate biomarkers showing a higher signal for EVs isolated from liver-derived tissue samples (healthy and HCC tissue) in comparison to the non-target tissue types and the buffer control passed the validation and were taken forward for detection in plasma samples.

Those biomarkers which did not result in any significant differences in the signal of the CD63 surface marker on EVs between liver tissue, HCC tissue, kidney tissue, lung tissue and the buffer control did not pass the validation.

### Detection of the Candidate Biomarkers in Plasma Samples

EVs were collected from human plasma derived from blood taken from healthy individuals. EVs were then captured and isolated by targeting the different candidate biomarkers identified from the tissue sample experiments described above.

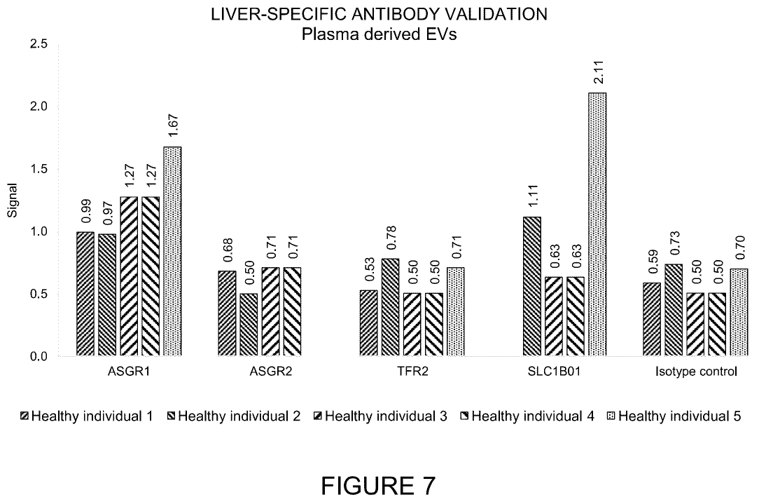

As shown in FIG. 7, the capture of EVs using magnetic beads pre-coated with anti-ASGR1 antibodies (ab1), anti-ASGR2 antibodies, anti-TFR2 antibodies or anti-SLCO1B1 antibodies resulted in a detectable signal in plasma samples at least once. It was also observed that marker expression varied among individuals.

From these results, ASGR1, ASGR2, TFR2 and SLCO1B1 were identified as candidate biomarkers of liver tissue.

EVs collected from human plasma were then captured and isolated using a cocktail of binding agents validated in the previous steps. The cocktail contained magnetic beads pre-coated with the anti-ASGR1 antibodies, the anti-ASGR2 antibodies, the anti-SLCO1B1 antibodies and the anti-TFR2 antibodies at a final ratio of 4:2:2:2.

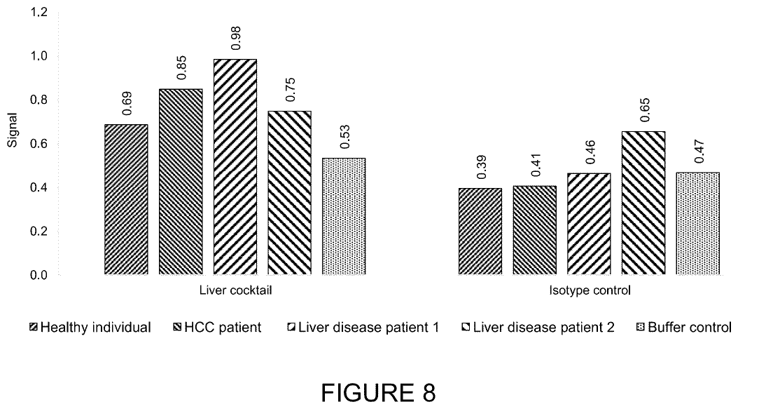

As shown in FIG. 8, the capture of EVs using the cocktail of antibodies targeting the identified biomarkers ASGR1, ASGR2, TFR2 and SLCO1B1 consistently detected a higher signal in human plasma from individuals with different phenotypes (healthy, HCC and liver disease) in comparison to the isotype control.

### Validation of Detection Approach

EVs were collected from different tissues and were subsequently captured and isolated by targeting the cell surface antigen CD63. Biotinylated anti-CD63 antibodies were used for signal detection.

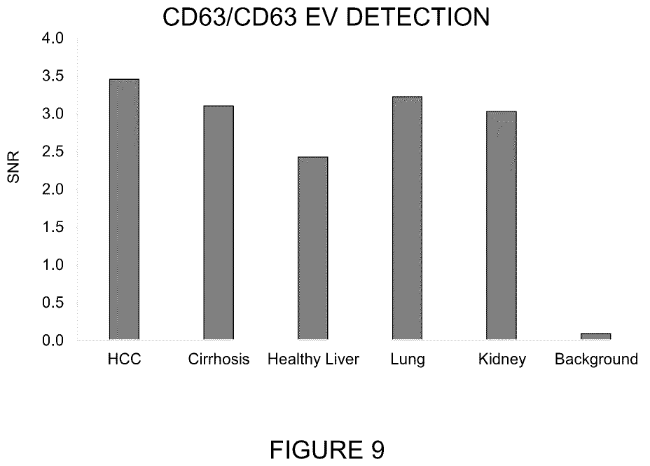

As shown in FIG. 9, the signal-to-noise ratio (SNR) of the detection of anti-CD63-labelled-magnetic bead-captured EVs using biotinylated anti-CD63 antibodies was comparable for different tissue types. This suggests that CD63 expression is similar on EVs obtained from different tissues.

This result demonstrates that any differences in detection signal observed following the capture of EVs using magnetic beads labelled with antibodies targeting other biomarkers is dependent on their differential capture of the EVs.

### Example 2: Mass-Spectrometry Validation of Liver-Derived EVs Isolation Method

### Materials and Methods:

Collection of EVs from Human Plasma

Frozen human plasma was acquired and EVs collected according to Example 1-3).

Isolation of Hepatocyte-Derived EVs 5.4 mL isolated plasma EVs were diluted in 20% Assay Defender (Candor Bioscience) and captured with 300 μL of the cocktail of binding agents selected in Example 1-3) (a cocktail of magnetic beads pre-coated with anti-ASGR1 antibodies, anti-ASGR2 antibodies, anti-SLCO1B1 antibodies and anti-TFR2 antibodies at a final ratio of 4:2:2:2). Incubation occurred for 16 hrs at 4° C. and mixed every 30 min in a KingFisher.

EVs which were captured by the antibody-labelled-magnetic beads were recovered and other non-captured EVs and plasma particles were washed away in a magnetic field using the KingFisher. Finally, captured EVs were lysed either for protein isolation or RNA isolation.

### Preparation of Antibody-Oligo-Labelled-Magnetic Beads

Antibodies selected in Example 1-3) were functionalised with DBCO-PEG4-NHS ester. In 100 μL of PBS, 20 μg of antibody was incubated with 1.54 nmol of DBCO at room temperature for 1 hr. Free DBCO was washed through a 12 kDa D-tube Dialyzer. Then, DBCO functionalized antibody was coupled with 8 μg oligo poly(A)-azide by incubation overnight at 4° C. Free oligo was removed with a 40 kDA Zeba column. Magnetic beads (Dynabeads® Oligo (dT) 25) were then labelled with the oligo-antibody with a ratio of 1 mg magnetic beads to 10 μL of oligo-antibody. The oligo-magnetic beads were prepared following the instructions on the kit.

### Elution of Intact Extracellular Vesicles

After isolation of the EVs, 300 μL of 0.2 M glycine 2.5 pH was added to the MBs for 10 min at room temperature and the supernatant was saved. This step was repeated once. Next, the magnetic beads were incubated in 300 UL in 0.05% T20 in Milli-Q at 70° C. for 5 min. All the resulting supernatants were pooled together.

If isolation of the EVs was performed with oligo-magnetic beads, the EVs were eluted by incubating 7.5 U of DNase I in 300 μL of 2.5 mM MgCl₂ with the magnetic beads at 37° C. for 1 hr.

### Lysis of Captured EVs

After isolation, 1× RIPA was added to lyse the EVs bound to the antibody-labelled-magnetic beads or to the eluted EVs for 1 hr at 4° C. followed by 10 min at 95° C., 1 min off HIFU (100% amplitude), 2×2 min in a tissue lyser with glass beads at 30 Hz followed by an additional heating step at 95° C. for 10 min. Alternatively, samples were lysed with sodium deoxycholate (SDC) commercially available buffer. Samples were then heated for 95° C. for 10 min with mixing at 1000 rpm in the shaker and the lysates retrieved.

### Sample Preparation for Mass Spectrometry

Proteins were precipitated in Trichloroacetic acid (5% TCA) and dissolved in 10 mM Tris/2 mM CaCl2, pH 8.2 followed by reduction and alkylation with TCEP and chloroacetamide, 30 min at 30° C. Protein digestion was performed with trypsin (100 ng/μL in 10 mM HCL), overnight at 37° C. The digested samples were dissolved in 12 μL ddH2O+0.1% formic acid and transferred to autosampler vials for Liquid chromatography-mass spectrometry analysis (LC-MS/MS). The samples were injected on an M-class UPLC coupled to a Fusion Lumos mass spectrometer (Thermo) or on a timsTOF-SCP (Bruker). Data was processed in a data-dependent acquisition (DDA) format.

The identification and quantification of proteins was performed using two search engine platforms: Mascot search engine (Matrixscience) and MaxQuant (MQ).

### Mass Spectrometry Analysis

For data analysis of the acquisition with the Fusion Lumos, total spectrum count was obtained using Scaffold 5, with the following stringency: Total spectrum count>1; Protein False Discovery Rate (FDR): 1%; Minimum number of peptides per protein: 2; Peptide FDR: 0.1%. The proteins identified with these cut-offs were matched against the list of 412 liver specific proteins obtained from The Human Protein Atlas in Example 1-1) (Table 1) to evaluate the efficiency of our method in capturing liver proteins in a complex sample. GO Enrichment Analysis was performed using the The Gene Ontology Resource.

For data analysis of the acquisition with the timsTOF-SCP, LFQ intensity data was obtained from the MaxQuant output file. Protein groups identified and with quantifiable LFQ intensity data were matched against our list of 412 liver proteins and 16 proteins known to be fundamental or associated for the biogenesis of EVs and exosomes, namely: ATP1A1, BSG, CAV1, CAV2, CD47, CD63, CD81, CD9, CLTC, ITGB1Bp, LAMP1, LGALS3BP, PDCD6IP, SDCBP, SLC3A2 and TSG101.

Log2 transformed LFQ intensity values, of the protein groups that matched both lists, were plotted with RStudio, Protein groups identified and with quantifiable LFQ intensity data were used for functional enrichment on DAVID, the CC (cellular component) GO-Terms with higher statistical significance of association (Benjamini-Hochberg adjusted p-value<1E-10) were used to originate ThreeMap figures using Revigo.

### Results:

### Orbitrap Mass Analyser

EVs collected from human plasma were captured and isolated using the cocktail of binding agents selected in Example 1-3). The captured EVs were eluted using (1) glycine, (2) RIPA, or (3) DNase and the lysate was subsequently processed and analysed by mass spectrometry with the Fusion Lumos, as described above.

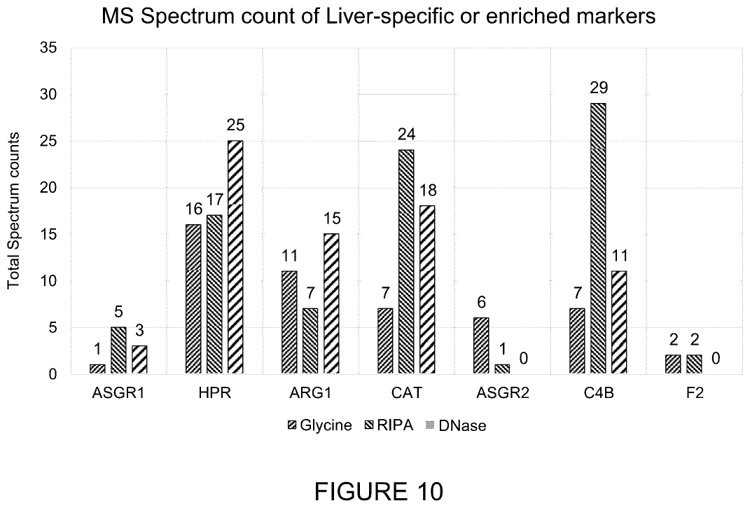

As shown in FIG. 10, a total of 7 liver-specific markers were detected by mass spectrometry: ASGR1, HPR, ARG1, CAT, ASGR2, C4B, and F2.

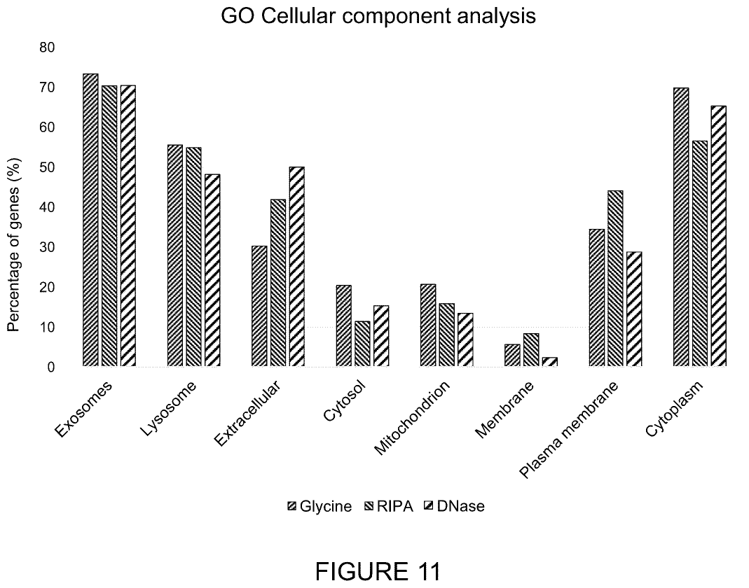

Furthermore, as shown in FIG. 11, independent of the protocol for lysis, EVs were identified as enriched following annotation of the genes identified by mass spectrometry using GO cellular component analysis.

These results demonstrate that liver-derived EVs were enriched following the isolation method described above.

### TIMS-TOF Mass Technology

EVs collected from human plasma were captured and isolated using the cocktail of binding agents selected in Example 1-3). The captured EVs were lysed using (1) RIPA, OR (2) SDC buffer, and the lysate was subsequently processed and analysed by mass spectrometry with the timsTOF-SCP, as described above.

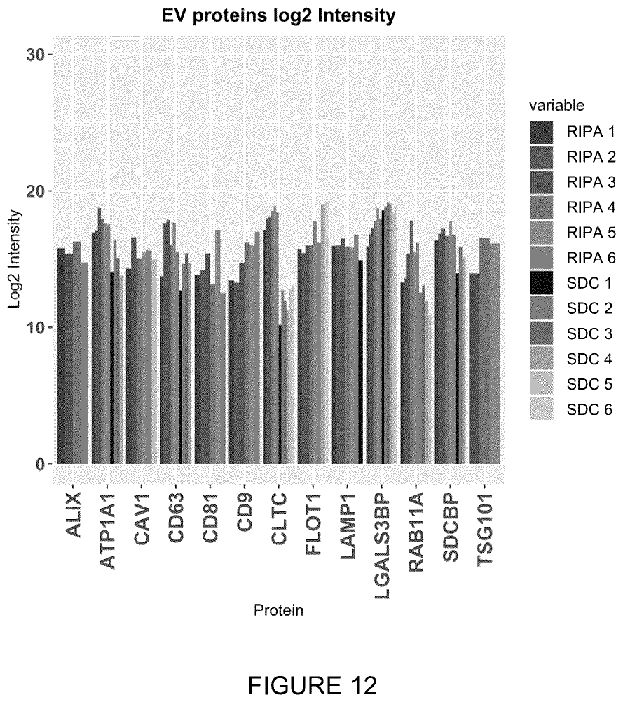

As shown in FIG. 12, a total of 13 classical EV markers were detected by mass spectrometry out of 16 proteins searched.

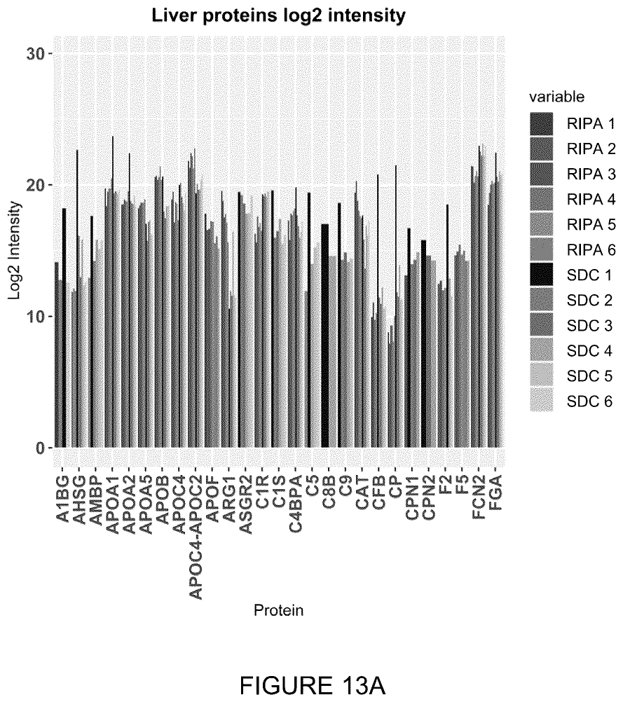

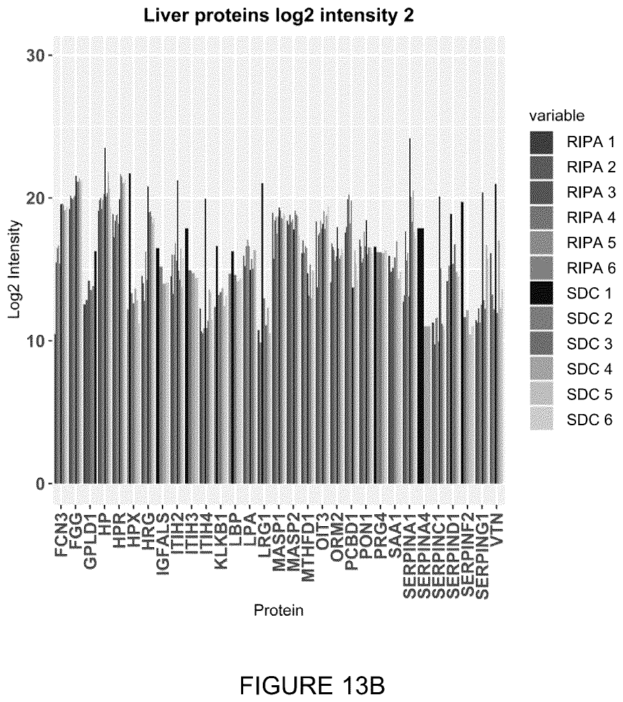

As shown in FIG. 13, a total of 63 liver-specific markers were detected by mass spectrometry.

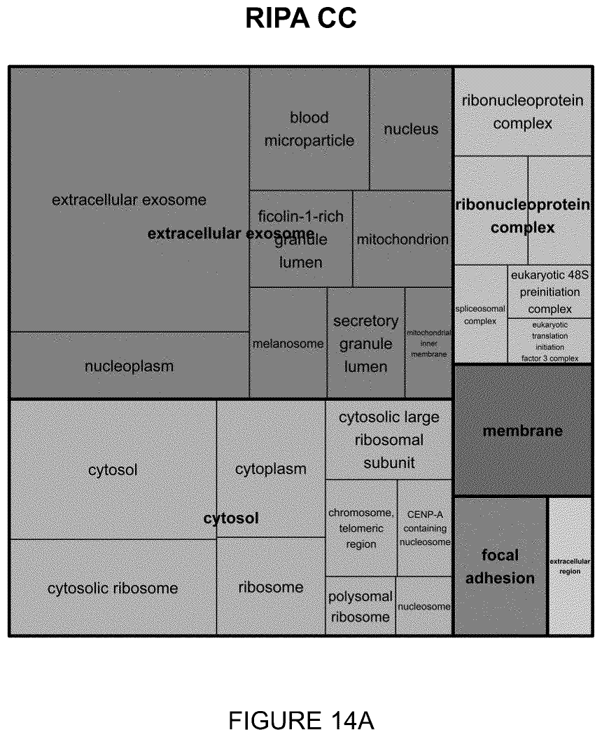

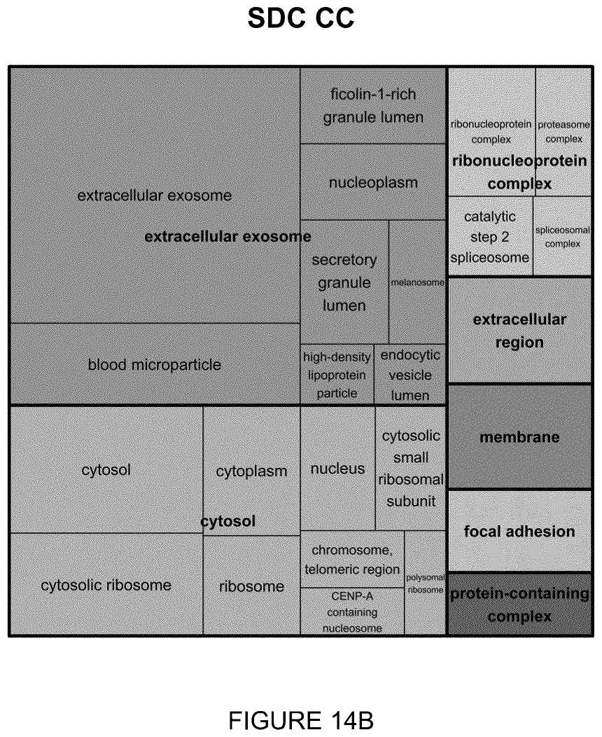

Furthermore, as shown in FIG. 14, independent of the protocol for lysis, components annotated as from the extracellular exosome were identified as enriched following annotation of the protein groups identified by mass spectrometry using GO cellular component analysis.

These results further demonstrate that liver-derived EVs were enriched following the isolation method described above.

### Example 3: RNA Sequencing Validation of Liver-Derived EV Isolation Method

### Materials and Methods:

Collection of EVs from Human Plasma

Frozen human plasma from healthy volunteers, Cirrhosis patients and HCC patients was acquired and EVs collected according to Example 1-3).

### Isolation of Hepatocyte-Derived EVs

5.4 mL isolated plasma EVs were diluted in 20% Assay Defender (Candor Bioscience) and captured with 300 μL of magnetic beads pre-coated with anti-ASGR1 antibodies alone, as prepared according to Example 1-2), or with 300 μL of the cocktail of binding agents (TOP4) selected in Example 1-3) (a cocktail of magnetic beads pre-coated with anti-ASGR1 antibodies, anti-ASGR2 antibodies, anti-SLCO1B1 antibodies and anti-TFR2 antibodies at a final ratio of 4:2:2:2). For RNA analysis, 0.75 mL of isolated plasma EVs were used in 20% Assay Defender with 35 μL of magnetic beads coated with the antibodies described above. Incubation occurred for 16 hrs at 4° C. and mixed every 30 min in a KingFisher.

EVs which were captured by the antibody-labelled-magnetic beads were recovered and other non-captured EVs and plasma particles were washed away in a magnetic field using the KingFisher. Finally, RNA was isolated from the captured EVs.

RNA Isolation from Captured EVs

After isolation, 600 μL of PBS were added to the EVs bound to the antibody-labelled-magnetic beads and the qEV RNA extraction Kit from IZON was used for RNA isolation according to the supplier instructions. In short, samples were lysed for 20 min and the antibody-labelled-magnetic beads were then removed from solution. The RNA was ethanol precipitated and loaded to a spin-column. The column was washed two times before doing elution of RNA with the elution buffer provided in 50 μL. Samples were speed-vacuum dried at 45° C. and resuspended in 10 μL of nuclease free water and stored at −80° C. if not immediately used.

### Small RNA Library Construction

Small RNA libraries were prepared with the PerkinElmer NEXTFLEX V4 kit and the manufacturer's instructions were followed. The starting material was 4 μL of isolated RNA as described before with the addiction of 1 μL of tRNA/YRNA blocker. The quality of the library construction was accessed with a High Sensitivity D1000 DNA ScreenTape analysis in TapeStation 4200, while its concentration was measured with the Qubit dsDNA High Sensitivity Assay.

### RNA Sequencing and Data Analysis

Sequencing was performed on a NextSeq 550 or NextSeq 2000 Illumina instrument with 1×75 or 1×100 Cycles, respectively. Small RNA reads were subject to QC and annotation via the pipeline miRge3.0 (Wickham, 2016) via parameters with the following flags activated;—on human-ie-ai-spl and with appropriate adaptors supplied for removal. Post-annotation processing was performed using Rstudio (Patil and Halushka, 2021) (for presence/absence analysis, differential expression analysis and results visualisation) and python version 3 (for relevance investigations). Presence and absence analysis was performed using simple set operations. Differential expression was performed using DESeq2 with settings mirroring those supplied for miRge3.0 differential expression analysis. Relevance investigation was performed using custom BioNLP python code and the results visualised using ggplot2 (R Core Team, 2023).

### Results:

### Comparison of Liver-Derived EV Isolation Using the ASGR1 Binding Agent Vs the Cocktail of Binding Agent by RNAseq

EVs collected from human plasma acquired from healthy volunteers were pooled in equal volumes and then captured and isolated using anti-ASGR1 antibody-labelled-magnetic beads or the cocktail of binding agents selected in Example 1-3). After EVs and small

RNAseq isolation, the samples were evaluated using two different RNA library preparation methods, namely RealSeq (Sequencing Method 1) and NEXTFLEX v4 (Sequencing Method 2) in two independent runs for each. The number of total miRNA IDs obtained were analysed.

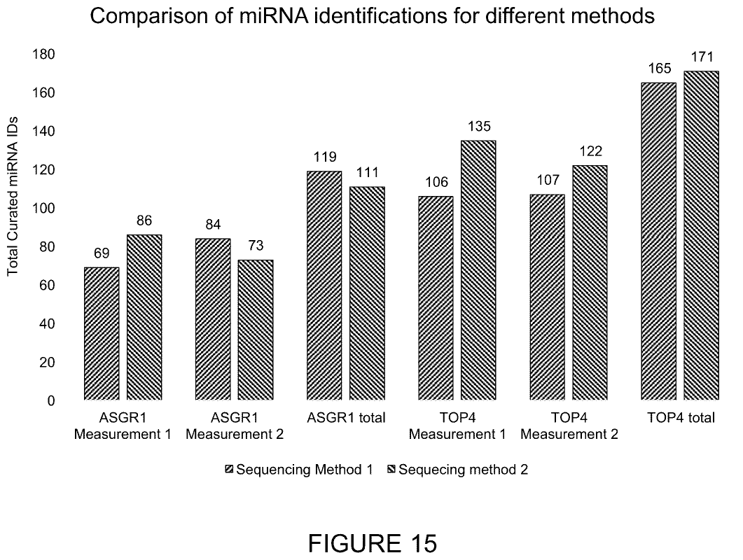

As shown in FIG. 15, independent of the method of RNA Sequencing method used, the isolation of EVs using the cocktail of binding agents which target ASGR1, ASGR2, SLCO1B1 and TFR2 in combination (TOP4) resulted in a higher number of annotated miRNA IDs being detected in a small sample in comparison to using a binding agent targeting ASGR1 alone.

This result demonstrates that using the cocktail of binding agents provides an advantage in capturing and identifying miRNAs from liver-derived EVs than by using a binding agent targeting the single biomarker ASGR1 alone.

### Comparison of Liver-Derived EV Isolation and Previous Methods

Plasma was obtained from 8 HCC patients, 8 Cirrhosis patients and 4 healthy volunteers. EVs were then collected and were pooled in two equal volumes. Group A was directly processed for RNA sequencing (Protocol A). While, Group B was then further captured and isolated using the cocktail of binding agents selected in Example 1-3) before being processed for RNA sequencing (Protocol B).

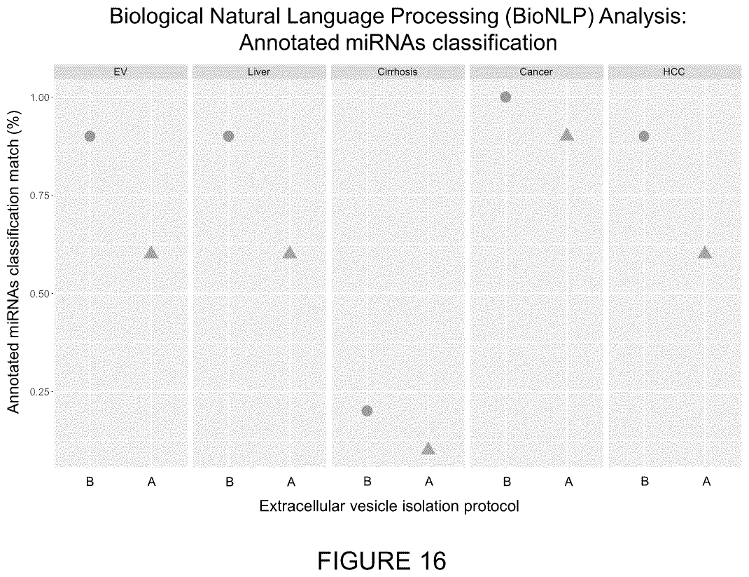

As shown in FIG. 16, Biological Natural Language Processing (BioNLP) analysis of the RNA sequencing data revealed that Protocol B substantially enriched for annotated miRNA information related to all key search terms (extracellular vesicles (EV), Liver, Cirrhosis, Cancer and HCC) in comparison to Protocol A.

This result demonstrates that using the isolation method of the present invention to capture liver-derived EVs is indeed enriching for a population of EVs derived from the healthy liver, as well as EVs derived from the liver of patients with HCC.

This result also demonstrates that using the isolation method of the present invention to capture liver-derived EVs provides a method for obtaining additional information from the EVs than using previous isolation protocols.

### LITERATURE REFERENCES

Chang et al. (2019). Role of miR-16-5p in the proliferation and metastasis of hepatocellular carcinoma. European Review for Medical and Pharmacological Sciences, 23 (1), 137-145.
Dias et al. (2024). An electro-optical technology for the ultrasensitive detection of small extracellular vesicle sub-populations and their protein epitope counts. ISCIENCE.
Hoshino at el. (2020). Extracellular Vesicle and Particle Biomarkers Define Multiple Human Cancers. Cell, 182 (4): 1044-1061.
Karimi et al. (2022). Tetraspanins distinguish separate extracellular vesicle subpopulations in human serum and plasma-Contributions of platelet extracellular vesicles in plasma samples. Journal of Extracellular Vesicles, 11, e12213.
Khan et al. (2020). Artificial Blood: The History and Current Perspectives of Blood Substitutes. Discoveries (Craiova), 8 (1), e10⁴.
Lekchnov et al. (2018). Searching for the Novel Specific Predictors of Prostate Cancer in Urine: The Analysis of 84 miRNA Expression. International Journal of Molecular Sciences, 19 (12, 4088.
Li et al. (2020). EV-origin: Enumerating the tissue-cellular origin of circulating extracellular vesicles using exLR profile. Computational and Structural Biotechnology Journal, 18, 2851-285.
Lightbody et al. (2021). Induction of microRNA hsa-let-7d-5p, and repression of HMGA2, contribute protection against lipid accumulation in macrophage ‘foam’ cells. Biochimica et Biophysica Acta (BBA)-Molecular and Cell Biology of Lipids, 1866 (11), 159005.
Moirangthem et al. (2023). Extracellular vesicle-mediated miR-126-3p transfer contributes to inter-cellular communication in the liver tumor microenvironment. International Journal of Oncology, 62 (2), 31.
Patil and Halushka (2021). miRge3. 0: a comprehensive microRNA and tRF sequencing analysis pipeline. NAR Genomics and Bioinformatics, 3 (3).
R Core Team (2023). R: A language and environment for statistical computing. R Foundation for Statistical Computing, Vienna, Austria. Available at: https://www.R-project.org/.
Sun et al. (2020). Purification of HCC-specific extracellular vesicles on nanosubstrates for early HCC detection by digital scoring. Nature Communications, 11, 4489.
Sun et al. (2023). HCC EV ECG score: An extracellular vesicle-based protein assay for detection of early-stage hepatocellular carcinoma. Hepatology, 77 (3), 774-788.
Tauro et al. (2012). Comparison of ultracentrifugation, density gradient separation, and immunoaffinity capture methods for isolating human colon cancer cell line LIM1863-derived exosomes. Methods, 56 (2), 293-304.
Weng, Z. (2021). Therapeutic roles of mesenchymal stem cell-derived extracellular vesicles in cancer. Journal of Hematology & Oncology, 14, 136.
Wickham, H. (2016). ggplot2: Elegant Graphics for Data Analysis. Springer-Verlag, New York.
Zheng, Q. (2021). The Unique Immunomodulatory Properties of MSC-Derived Exosomes in Organ Transplantation. Frontiers in Immunology, 12:659621.

1. A method of identifying a biomarker present at elevated levels in tissue of a defined category in an organism in comparison to the level in at least one different category of tissue, wherein the biomarker is detectable on or in extracellular vesicles secreted from cells of tissue of the defined category, the method comprising the steps of:
i. identifying a candidate biomarker;
ii. providing a binding agent which is capable of binding specifically to the candidate biomarker identified in step i.;
iii. detecting the presence of the candidate biomarker on an extracellular vesicle obtained from a sample of tissue of the defined category by binding the binding agent to the candidate biomarker in the sample wherein extracellular vesicles from tissue of the defined category preferentially bind the binding agent compared with extracellular vesicles from at least one different category of tissue from the organism;
iv. selecting the candidate biomarker as a biomarker present at elevated levels in tissue of the defined category.

2. A method according to claim 1, wherein step iii. comprises detecting the presence of the candidate biomarker using a technique capable of detecting the biomarker at a concentration of at least 1×10⁷ biomarkers/ml, preferably at least 1×10⁶, 1×10⁵, 1×10⁴, 1×10³, 1×10², 10 or 1 biomarkers/ml.

3. A method according to claim 1, wherein step iii. comprises detecting the presence of the candidate biomarker using a technique capable of detecting, by using the candidate biomarker, extracellular vesicles at a concentration of at least 1×10⁷ extracellular vesicles/ml, preferably at least 1×10⁶, 1×10⁵, 1×10⁴, 1×10³, 1×10², 10 or 1 extracellular vesicles/ml.

4. A method according to claim 1, wherein step iii. further comprises determining the concentration of the biomarker in the sample.

5. A method according to claim 1, wherein:
step i. comprises identifying a plurality of candidate biomarkers;
step ii. comprises providing a plurality of binding agents, each binding agent being capable of binding specifically to a respective candidate biomarker identified in step i;
step iii. comprises detecting the presence of each of the candidate biomarkers on an extracellular vesicle obtained from a sample of tissue of the defined category by binding the binding agents to the respective candidate biomarkers in the sample wherein extracellular vesicles from tissue of the defined category preferentially bind a combination of the plurality of binding agents compared with extracellular vesicles from the at least one different category of tissue from the organism; and
step iv. comprises selecting the plurality of candidate biomarkers as a combination of biomarkers present at elevated levels in tissue of the defined category.

6. A method according to claim 1, wherein step iii. further comprises contacting extracellular vesicles from the at least one different category of tissue with the or each binding agent and detecting binding of the extracellular vesicles from the at least one different category of tissue to the or each binding agent.

7. A method according to claim 1, wherein extracellular vesicles from tissue of the defined category preferentially bind the or each binding agent compared with extracellular vesicles from at least 2 different categories of tissue from the organism.

8. A method according to claim 1, wherein extracellular vesicles from tissue of the defined category preferentially bind the or each binding agent compared with extracellular vesicles from all other categories of tissue from the organism.

9. A method according to claim 1, wherein step iii. further comprises the step of detecting the presence of the or each candidate biomarker on an extracellular vesicle obtained from a cultured cell line which is representative of tissue of the defined category.

10. A method according to claim 9, wherein the extracellular vesicles obtained from a cultured cell line which is representative of tissue of the defined category preferentially bind the binding agent compared with extracellular vesicles obtained from a cultured cell line which is representative of at least one different category of tissue from the organism.

11. A method according to claim 9, wherein the extracellular vesicles obtained from the cultured cell line is in plasma which has been spiked with the extracellular vesicles.

12. A method according to claim 1, wherein the extracellular vesicle obtained from a sample of tissue is in plasma which has been spiked with the extracellular vesicles after the extracellular vesicles has been obtained from the sample of tissue.

13. A method according to claim 1, wherein step i. comprises the steps of:
1) identifying potential candidate biomarkers expressed in the defined category of tissue;
2) identifying potential candidate biomarkers which are present in the defined category of tissue at a higher level than in at least one different category of tissue from the organism;
3) identifying potential candidate biomarkers which are transmembrane or surface proteins;
4) identifying potential candidate biomarkers which are present in extracellular vesicles; or
5) a combination of steps 1) to 4) performed in any order, and thereby identifying a candidate biomarker.

14. A method according to claim 1, wherein step i. comprises the steps of:
1) identifying potential candidate biomarkers expressed in the defined category of tissue;
2) identifying potential candidate biomarkers from within the potential candidate biomarkers identified in step 1) which are present in the defined category of tissue at a higher level than in at least one different category of tissue from the organism;
3) identifying potential candidate biomarkers from within the potential candidate biomarkers identified in step 2) which are transmembrane or surface proteins.
4) validating a candidate biomarker from within the potential candidate biomarkers identified in step 3) which is present in extracellular vesicles or validating candidate biomarkers from within the potential candidate biomarkers identified in step 3) which are present in extracellular vesicles.

15. A method according to claim 1, wherein step iii. further comprises the step of fractionating a precursor sample of tissue of the defined category enriched in extracellular vesicles in order to provide the sample of tissue of the defined category.

16. A method according to claim 1, wherein the candidate biomarker is a transmembrane or surface protein.

17. A method according to claim 1, wherein the candidate biomarker is a cytosolic protein.

18. A method according to claim 1, wherein the candidate biomarker is a protein having a post-translational modification.

19. A method according to claim 1, wherein the candidate biomarker is a DNA molecule, an RNA molecule, a lipid or a post-translation modification.

20. A method according to claim 1, wherein the or each binding agent comprises an antibody, an aptamer, a lectin, a lipid-binding protein or domain, or a DNA or RNA primer or probe.

21. A method according to claim 1, wherein, in step iii. the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene.

22. A method according to claim 1, wherein the defined category of tissue is liver tissue, preferably wherein the cells from which the extracellular vesicles are secreted are hepatocytes, Kupffer cells, stellate cells or liver resident dendritic cells.

23. A method according to claim 22, wherein the or each biomarker present at elevated levels in tissue of the defined category is selected from the group consisting of ASGR1, ASGR2, TFR2, SLCO1B1, SLC38A3, TMEM56, UNC93A, SLC22A9, SLC2A2, and FXYD1, or a combination thereof, preferably wherein the or each biomarker present at elevated levels in tissue of the defined category is selected from the group consisting of ASGR1, ASGR2, TFR2, and SLCO1B1, or a combination thereof.

24. A method according to claim 23, wherein the or each biomarker present at elevated levels in tissue of the defined category consists of ASGR1, ASGR2, TFR2, and SLCO1B1.

25. A method according to claim 23, wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

26. A method according to claim 25, wherein each binding agent is provided in a proportion of 10-50% of the total, wherein the total mixture of binding agents is equal to 100% or wherein each binding agent is provided in equal proportions.

27. A method according to claim 25, wherein the binding agent comprises a plurality of binding agents consisting of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof, and wherein the anti-ASGR1 antibody or antigen binding fragment thereof is provided in a proportion of 30-50% of the total, and each of the other antibodies or antigen binding fragments thereof is provided in a proportion of 10-30% of the total, wherein the total mixture of the binding agents is equal to 100%.

28. A method according to claim 1, wherein the defined category of tissue is blood tissue.

29. A method according to claim 1, wherein the biomarker is specific for tissue of the defined category or wherein the combination of biomarkers is specific for tissue of the defined category.

30. A method according to claim 1, wherein step iii. further comprises the step of detecting the binding of the or each binding agent to the respective candidate biomarker on or in an extracellular vesicle.

31. A method according to claim 1, wherein the sample of tissue is healthy tissue or diseased tissue.

32. A method of enriching a biological sample comprising a mixture of extracellular vesicles comprising the steps of:
a) identifying one or more biomarkers present at elevated levels in a tissue by performing the method of claim 1; and
b) capturing extracellular vesicles in the biological sample, wherein the extracellular vesicles display the or each biomarker identified in step a), to generate an enriched fraction of extracellular vesicles from tissue of the defined category.

33. A method according to claim 32, wherein the mixture of extracellular vesicles comprises extracellular vesicles from tissue of the defined category and extracellular vesicles from at least one different category of tissue from the organism.

34. A method according to claim 32, wherein the extracellular vesicles are captured in step b) using one or more binding agents which are capable of binding specifically to the or each biomarker.

35. A method according to claim 32, wherein step b) further comprises isolating the extracellular vesicles displaying the or each biomarker identified in step a) which are captured using the or each biomarker.

36. A method according to claim 35, wherein the isolated extracellular vesicles are intact extracellular vesicles.

37. A method according to claim 32, wherein the method further comprises the step of releasing the contents of the captured or isolated extracellular vesicles.

38. A method of analysing a biological sample comprising a mixture of extracellular vesicles comprising the steps of:
a) identifying one or more biomarkers present at elevated levels in a tissue by performing the method of claim 1;
b) capturing extracellular vesicles in the biological sample, wherein the extracellular vesicles display the or each biomarker identified in step a), to generate an enriched fraction of extracellular vesicles; and
c) analysing the contents of an extracellular vesicle in the enriched fraction obtained in step b).

39. A method according to claim 387, wherein the mixture of extracellular vesicles comprises extracellular vesicles from tissue of the defined category and extracellular vesicles from at least one different category of tissue from the organism.

40. A method according to claim 38, wherein the extracellular vesicles are captured in step b) using one or more binding agents which are capable of binding specifically to the or each biomarker.

41. A method according to claim 38, wherein step b) further comprises isolating the extracellular vesicles displaying the or each biomarker identified in step a) which are captured using the or each biomarker.

42. A method according to claim 41, wherein the isolated extracellular vesicles are intact extracellular vesicles.

43. A method according to claim 38, wherein the method further comprises the step of releasing the contents of the captured or isolated extracellular vesicles.

44. A method according to claim 32, wherein the biological sample is a biofluid, preferably blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

45. A method according to claim 32, wherein step b) comprises capturing extracellular vesicles in the biological sample using the or each binding agent.

46. A method of enriching a biological sample comprising a mixture of extracellular vesicles comprising the step of:
capturing extracellular vesicles in the biological sample, wherein the extracellular vesicles display one or more biomarkers, to generate an enriched fraction of extracellular vesicles,
wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

47. A method of analysing a biological sample comprising a mixture of extracellular vesicles comprising the steps of:
a) capturing extracellular vesicles in the biological sample, wherein the extracellular vesicles display one or more biomarkers, to generate an enriched fraction of extracellular vesicles; and
b) analysing the contents of an extracellular vesicle in the enriched fraction obtained in step a),
wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

48. A method according to claim 46 or 47, wherein the step of capturing extracellular vesicles comprises capturing extracellular vesicles based on the extracellular vesicles displaying one or more biomarkers and wherein the one or biomarkers consist of ASGR1, ASGR2, TFR2, and SLCO1B1.

49. A method according to claim 46, wherein the extracellular vesicles are captured using one or more binding agents which are capable of binding specifically to the or each biomarker.

50. A method according to claim 46, wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

51. A method according to claim 50, wherein each binding agent is provided in a proportion of 10-50% of the total, wherein the total mixture of binding agents is equal to 100% or wherein each binding agent is provided in equal proportions.

52. A method according to claim 50, wherein the binding agent comprises a plurality of binding agents consisting of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof, and wherein the anti-ASGR1 antibody or antigen binding fragment thereof is provided in a proportion of 30-50% of the total, and each of the other antibodies or antigen binding fragments thereof is provided in a proportion of 10-30% of the total, wherein the total mixture of the binding agents is equal to 100%.

53. A method according to claim 46, wherein the mixture of extracellular vesicles comprises extracellular vesicles from tissue of a defined category and extracellular vesicles from at least one different category of tissue from the organism, and wherein the or each biomarker is present at elevated levels in the tissue of the defined category.

54. A method according to claim 46, wherein the method further comprises isolating the extracellular vesicles displaying the or each biomarker which are captured using the or each biomarker.

55. A method according to claim 54, wherein the isolated extracellular vesicles are intact extracellular vesicles.

56. A method according to claim 46, wherein the method further comprises the step of releasing the contents of the captured or isolated extracellular vesicles.

57. A method according to claim 46, wherein the biological sample is a biofluid, preferably blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

58. A method according to claim 46, wherein the step of capturing extracellular vesicles in the biological sample comprises capturing extracellular vesicles in the biological sample using the or each binding agent.

59. Use of one or more biomarkers displayed on extracellular vesicles to generate an enriched fraction of extracellular vesicles, wherein the enriched fraction of extracellular vesicles is secreted from tissue of a defined category in an organism, wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

60. A use according to claim 59, wherein the biomarkers consist of ASGR1, ASGR2, TFR2, and SLCO1B1.

61. A use according to claim 59, wherein the enriched fraction of extracellular vesicles is enriched from a biological sample comprising a mixture of extracellular vesicles.

62. A use according to claim 61, wherein the mixture of extracellular vesicles comprises extracellular vesicles from tissue of the defined category and extracellular vesicles from at least one different category of tissue from the organism.

63. A use according to claim 61, wherein the biological sample is a biofluid, preferably blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

64. Use of one or more binding agents to generate an enriched fraction of extracellular vesicles, wherein the enriched fraction of extracellular vesicles is secreted from tissue of a defined category in an organism, wherein the or each binding agent is capable of binding specifically to a biomarker displayed on the extracellular vesicles, wherein the or each binding agent is selected from the group consisting of an anti-ASGR1 antibody or antigen binding fragment thereof, an anti-ASGR2 antibody or antigen binding fragment thereof, an anti-TFR2 antibody or antigen binding fragment thereof, an anti-SLCO1B1 antibody or antigen binding fragment thereof, an anti-SLC38A3 antibody or antigen binding fragment thereof, an anti-TMEM56 antibody or antigen binding fragment thereof, an anti-UNC93A antibody or antigen binding fragment thereof, an anti-SLC22A9 antibody or antigen binding fragment thereof, an anti-SLC2A2 antibody or antigen binding fragment thereof, and an anti-FXYD1 antibody or antigen binding fragment thereof, or a combination thereof, preferably an anti-ASGR1 antibody or antigen binding fragment thereof, an anti-ASGR2 antibody or antigen binding fragment thereof, an anti-TFR2 antibody or antigen binding fragment thereof, or an anti-SLCO1B1 antibody or antigen binding fragment thereof, or a combination thereof.

65. A use according to claim 64, wherein the enriched fraction of extracellular vesicles is enriched from a biological sample comprising a mixture of extracellular vesicles.

66. A method of detecting one or more biomarkers on or in extracellular vesicles secreted from a tissue of a defined category, wherein the or each biomarker is selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

67. A method according to claim 66, wherein the method comprises a plurality of biomarkers comprising ASGR1, ASGR2, TFR2, and SLCO1B1.

68. A method according to claim 66, wherein the or each biomarker is detected by the binding of one or more binding agents, wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, the or each biomarker is SLCO1B1 and the or each binding agent is an anti-SLCO1B1 antibody or antigen binding fragment thereof, the or each biomarker is SLC38A3 and the or each binding agent is an anti-SLC38A3 antibody or antigen binding fragment thereof, the or each biomarker is TMEM56 and the or each binding agent is an anti-TMEM56 antibody or antigen binding fragment thereof, the or each biomarker is UNC93A and the or each binding agent is an anti-UNC93A antibody or antigen binding fragment thereof, the or each biomarker is SLC22A9 and the or each binding agent is an anti-SLC22A9 antibody or antigen binding fragment thereof, the or each biomarker is SLC2A2 and the or each binding agent is an anti-SLC2A2 antibody or antigen binding fragment thereof, or the or each biomarker is FXYD1 and the or each binding agent is an anti-FXYD1 antibody or antigen binding fragment thereof, or a combination thereof, preferably wherein the or each biomarker is ASGR1 and the or each binding agent is an anti-ASGR1 antibody or antigen binding fragment thereof, the or each biomarker is ASGR2 and the or each binding agent is an anti-ASGR2 antibody or antigen binding fragment thereof, the or each biomarker is TFR2 and the or each binding agent is an anti-TFR2 antibody or antigen binding fragment thereof, or the or each biomarker is SLCO1B1 and the or each binding agent is an anti-SLCO1B1 antibody or antigen binding fragment thereof, or a combination thereof.

69. A method according to claim 66, wherein the or each biomarker is detected on or in extracellular vesicles secreted from a tissue of a defined category in a biological sample comprising a mixture of extracellular vesicles.

70. A kit for enriching a biological sample comprising a mixture of extracellular vesicles to generate an enriched fraction of extracellular vesicles, wherein the enriched fraction of extracellular vesicles is secreted from tissue of a defined category in an organism, wherein the kit comprises one or more binding agents which are capable of binding specifically to a biomarker present at elevated levels in tissue of the defined category, and wherein the or each binding agent is selected from the group consisting of an anti-ASGR1 antibody or antigen binding fragment thereof, an anti-ASGR2 antibody or antigen binding fragment thereof, an anti-SLCO1B1 antibody or antigen binding fragment thereof, an anti-SLC38A3 antibody or antigen binding fragment thereof, an anti-TMEM56 antibody or antigen binding fragment thereof, an anti-UNC93A antibody or antigen binding fragment thereof, an anti-SLC22A9 antibody or antigen binding fragment thereof, an anti-SLC2A2 antibody or antigen binding fragment thereof, and an anti-FXYD1 antibody or antigen binding fragment thereof, or a combination thereof, and optionally wherein the kit comprises an anti-TFR2 antibody or antigen binding fragment thereof, preferably an anti-ASGR1 antibody or antigen binding fragment thereof, an anti-ASGR2 antibody or antigen binding fragment thereof, or an anti-SLCO1B1 antibody or antigen binding fragment thereof, or a combination thereof, and optionally wherein the kit comprises an anti-TFR2 antibody or antigen binding fragment thereof.

71. A use according to claim 65, a method according to claim 69, or kit according to claim 70, wherein the mixture of extracellular vesicles comprises extracellular vesicles from tissue of the defined category and extracellular vesicles from at least one different category of tissue from the organism.

72. A use, method, or kit according to any one of claims 65, 69 or 70, wherein the biological sample is a biofluid, preferably blood, urine, saliva, lymph, bile, cerebrospinal fluid, phlegm, mucus, tears, Bronchoalveolar Lavage (BAL) fluid, earwax, sweat, faeces, breast milk, interstitial fluids, vaginal fluids, semen, gastric juice, blister fluid or cyst fluid.

73. A use, method, or kit according to any one of claims 64, 65, 69 or 70, wherein the or each binding agent consists of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof.

74. A use, method, or kit according to any one of claims 64, 65, 69 or 70, wherein the method, use or kit comprises a plurality of binding agents consisting of the anti-ASGR1 antibody, the anti-ASGR2 antibody, the anti-SLCO1B1 antibody and the anti-TFR2 antibody.

75. A use, method, or kit according to any one of claim 64, 65, 69 or 70, wherein the or each binding agent is provided attached to a substrate, preferably wherein the substrate comprises a magnetic bead or nanoparticle, a gold bead or nanoparticle, a polystyrene bead, an affinity chromatography column, a microplate, a microfluidics channel or a biochip with a surface made of gold, silicon oxide, glass, graphene or polystyrene.

76. A use, method, or kit according to any one of claims 64, 65, 69 or 70, wherein each binding agent is provided in a proportion of 10-50% of the total, wherein the total mixture of binding agents is equal to 100% or wherein each binding agent is provided in equal proportions.

77. A use, method, or kit according to any one of claims 64, 65, 69 or 70, wherein the use, method or kit comprises a plurality of binding agents consisting of the anti-ASGR1 antibody or antigen binding fragment thereof, the anti-ASGR2 antibody or antigen binding fragment thereof, the anti-SLCO1B1 antibody or antigen binding fragment thereof and the anti-TFR2 antibody or antigen binding fragment thereof, and wherein the anti-ASGR1 antibody or antigen binding fragment thereof is provided in a proportion of 30-50% of the total, and each of the other antibodies or antigen binding fragments thereof is provided in a proportion of 10-30% of the total, wherein the total mixture of binding agents is equal to 100%.

78. An enriched fraction of extracellular vesicles, the fraction being enriched with extracellular vesicles secreted from tissue of a defined category in an organism, which extracellular vesicles display one or more biomarkers selected from the group consisting of ASGR1, ASGR2, TFR2, SLCO1B1, SLC38A3, TMEM56, UNC93A, SLC22A9, SLC2A2, and FXYD1, or a combination thereof, preferably ASGR1, ASGR2, TFR2, and SLCO1B1, or a combination thereof.

79. An enriched fraction of extracellular vesicles according to claim 78, wherein the extracellular vesicles secreted from tissue of a defined category in an organism display a plurality of biomarkers comprising ASGR1, ASGR2, TFR2, and SLCO1B1.

80. A use, method, kit, or enriched fraction of extracellular vesicles according to any one of claim 59, 64, 65, 69 or 70, wherein the defined category of tissue is liver tissue.

81. A method of manufacturing the kit according to claim 70, wherein the method comprises colocating the or each binding agent.

82. A use, method, kit, or enriched fraction of extracellular vesicles according to claim 53, wherein the defined category of tissue is liver tissue.

83. A method of manufacturing the kit according to claim 80, wherein the method comprises colocating the or each binding agent.
